(19) United States
(12) Patent Application Publication
(10) Pub. No.: US 2026/0108560 A1
(43) Pub. Date: Apr. 23, 2026

(54) TRANSPLANTING STEM CELL-DERIVED MICROGLIA TO TREAT LEUKODYSTROPHIES

(71) Applicant: THE REGENTS OF THE UNIVERSITY OF CALIFORNIA, Oakland, CA (US)

(72) Inventors: Mathew Blurton-Jones, Irvine, CA (US); Hayk Davtyan, Irvine, CA (US); Jean Paul Chadarevian, Irvine, CA (US); Robert Spitale, Irvine, CA (US); Sunil Gandhi, Irvine, CA (US); Jonathan Hasselmann, Irvine, CA (US); Whitney England, Irvine, CA (US)

(21) Appl. No.: 18/993,901

(22) PCT Filed: Jul. 13, 2023
(86) PCT No.: PCT/US2023/070166

§ 371 Date: Jan. 13, 2025

Related U.S. Application Data
(60) Provisional application No. 63/388,766, filed on Jul. 13, 2022.

(51) Int. Cl. A61K 35/30; A61P 25/28; C07K 14/715; C12N 5/079; C12N 5/10; C12N 15/85

(52) U.S. Cl. CPC A61K 35/30; A61P 25/28; C07K 14/7153; C12N 5/0622; C12N 5/10; C12N 15/85; C12N 2506/45; C12N 2510/00

(57) ABSTRACT

The present disclosure provides myeloid cells, microglial progenitor cells, and microglia-like cells in which a mutant gene has been repaired or replaced, and methods of using such cells to treat disease. Mutations in CSF1R were corrected in human induced pluripotent stem cells. The resulting cells were differentiated into microglial progenitors and then transplanted into the brain of xenotransplantation-compatible CSF1R-AFIRE/AFIRE mice, thereby preventing or reversing phenotypes associated with leukodystrophy, including thalamic microbleeds, calcification, astrogliosis, axonal spheroids, synaptic loss, and accumulation of Tau phosphorylated at threonine residue 217.

### CROSS-REFERENCE

This application claims benefit of U.S. Provisional Application No. 63/388,766, filed Jul. 13, 2022, which is incorporated herein its entirety by reference.

### STATEMENT AS TO FEDERALLY SPONSORED RESEARCH

This present disclosure was made with government support under 1-RF1-DA048813-01 awarded by the National Institutes of Health, and R43-NS124409 Small Business Innovation Research. The government has certain rights in the disclosure.

### REFERENCE TO A SEQUENCE LISTING

The instant application contains a Sequence Listing which has been submitted electronically in XML format and is hereby incorporated by reference in its entirety. Said XML copy, created on Jul. 12, 2023, is named UCI2208_64465704601_SL.xml and is 21,540 bytes in size.

### FIELD OF THE DISCLOSURE

This disclosure relates to induced human microglial-like cells (iMGLs) or hematopoietic progenitor cells (HPCs), derived from pluripotent stem cells (PSCs), that include synthetic alleles of a human colony stimulating factor-1 receptor (CSF1R) gene or express at least one canonical microglial marker. This disclosure further relates to human iMGLs or HPCs, derived from PSCs, that may be used to treat diseases associated with a mutation of the CSF1R gene.

### BACKGROUND

Microglia, the primary immune cell of the brain, play critical roles in brain development and adult neurological function. As highly plastic cells, microglia also respond rapidly to brain pathologies and thus have been implicated in virtually every neurological disorder, including Alzheimer's disease (AD), Frontotemporal Dementia (FTD), and Amyotrophic Lateral Sclerosis (ALS). Microglia have also been implicated in several rare genetic diseases in which mutations in microglial expressed genes lead to alterations in the normal function of these important cells.

For example, leukodystrophies comprise a class of rare genetic disorders linked to gene abnormalities in microglia and lead to abnormal development or destruction of the myelin sheath in the nervous system or white matter in the brain. Two such “microgliopathies” belonging to the class of leukodystrophies are Adult-onset leukoencephalopathy with axonal spheroids and pigmented glia (ALSP) and Brain abnormalities, neurodegeneration, and dysosteosclerosis (BANDDOS), whereby both exhibit similar significant clinical and pathological phenotypes but each arises from different genetic etiologies.

ALSP is a progressive neurological disorder caused by dominantly inherited mutations in the gene CSF1R which is important for the function and survival of microglia. It is an autosomal dominant neurodegenerative disorder characterized by neuropsychiatric and motor impairments. Patients with ALSP typically develop initial signs of disease in their thirties or forties which manifest with an array of symptoms including personality and behavioral changes, progressive cognitive decline, depression, and motor disturbances. In contrast, BANDDOS involves homozygous or compound heterozygous mutations in CSF1R. BANDDOS symptoms generally occur at younger ages than ALSP, typically manifesting as a pediatric disease.

ALSP was previously referred to as Hereditary diffuse leukoencephalopathy with spheroids (HDLS) or as Pigmented orthochromatic leukodystrophy (POLD). ALSP is one example of leukodystrophy, a group of diseases that involve degeneration of the white matter and myelinated tracts within the brain and/or spinal cord. White matter pathology is often accompanied by other neuropathologies such as axonal spheroids, microgliosis, astrocytosis, and neuronal death. Together these neuropathologies lead to a progressive impairment in cognitive and/or motor function. While the precise symptoms, age of onset, and cause of leukodystrophies vary, many of these diseases involve mutations in specific genes. Interestingly, many of the genes implicated in leukodystrophies, including ALSP, are highly or even exclusively expressed by microglia within the brain. This finding has led some scientists to propose that several leukodystrophies can be considered primary microgliopathies.

ALSP arguably represents the clearest example of primary microgliopathy as it is caused by mutations in the colony stimulating factor-1 receptor (CSF1R), a gene that is needed for the survival, proliferation, and normal function of microglia. CSF1R mutations lead to an age-related decrease in and dysfunction of microglia, brain calcification, astrogliosis, white matter lesions, and axonal spheroids. Although ALSP incidence is relatively rare, with current estimates of 1-5 per million, many physicians and researchers who study the disease believe that it is most likely under-diagnosed, as several dozen cases in the literature have detailed a misdiagnosis as multiple sclerosis or frontotemporal degeneration.

ALSP and other primary microgliopathies are 100% fatal and there are no treatment solutions. Like most other hereditary leukoencephalopathies, there is currently no cure or effective therapeutic treatment for ALSP. Symptomatic treatments to reduce depression, muscle spasticity, and seizures are the current standard of care. Some patients have been treated with hematopoietic stem cell transplantation (HSCT) as an experimental therapeutic, resulting in partial stabilization of cognition and ambulatory function. Given that microglia are not originally derived from bone marrow, it is possible that macrophages derived from the donor migrated into the brain and provided some partial benefit. However, this approach requires preconditioning to ablate endogenous bone marrow hematopoietic stem cells which concurrently disrupts the blood-brain barrier potentially enabling macrophage migration into the brain, and long-term immunosuppression, and has failed to slow the neurological impairments in a larger study of lysosomal storage disorders, which also have microgliopathic components. A safer and more logical approach is needed.

The compositions and methods of this disclosure represent a significant departure from the current paradigm. For example, present invention describes the direct transplantation of engineered cells (e.g., induced hematopoietic progenitor cells (iHPCs), microglial progenitor cells (MPCs), or human microglial-like cells (iMGLs)) comprising a synthetic repaired or replaced CSF1R gene into the central nervous system (CNS; e.g., the brain or spinal cord) of a subject. Surprisingly, the engineered cells are capable of engrafting and migrating throughout the brain to populate a microglial niche, without the deliberate depletion of any microglia from the CNS.

### SUMMARY

Provided herein is an engineered cell comprising a synthetic, repaired, or replaced Colony Stimulating Factor 1 Receptor (CSF1R) gene. In some embodiments, the synthetic repaired or replaced CSF1R gene is a human CSF1R gene. In some embodiments, the engineered cell is a microglia-like cell (iMGL). In some embodiments, the engineered cell is a hematopoietic progenitor cell (HPC) or microglial precursor cell (MPC). In some embodiments, the engineered cell is an induced pluripotent stem cell (iPSC). In some embodiments, the engineered cell is a myeloid cell, a hematopoietic stem cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell. In some embodiments, the engineered cell is capable of differentiating into a macrophage or a monocyte. In some embodiments, the engineered cell is a human cell. In some embodiments, the engineered cell does not express a mutant CSF1R gene. In some embodiments, the synthetic repaired or replaced CSF1R gene comprises an inserted CSF1R coding sequence or fragment thereof located 3′ of the fms-intronic response element (FIRE). In some embodiments, the synthetic repaired or replaced CSF1R gene comprises a silent mutation. In some embodiments, the synthetic CSF1R gene further comprises a stop codon and a poly-A signal. In some embodiments, the synthetic CSF1R gene encodes a polypeptide having at least 90% sequence identity to the amino acid sequence of SEQ ID NO: 1 or a fragment thereof. In some embodiments, the synthetic CSF1R gene comprises the nucleic acid sequence of SEQ ID NO: 2. In some embodiments, the CSF1R expression in the engineered cell is at least 80% of the CSF1R expression level in an otherwise identical control cell having two native, wildtype CSF1R alleles. In some embodiments, the CSF1R tyrosine receptor kinase activity in the engineered cell is at least 80% of the CSF1R tyrosine receptor kinase activity in an otherwise identical control cell having two native, wildtype CSF1R alleles. In some embodiments, the engineered cell is not edited but instead comprises a wildtype gene (e.g., CSF1R gene) from a healthy donor.

Also described herein is a pluripotent stem cell comprising a CRISPR endonuclease and a guide RNA complementary to a sequence of the CSF1R gene. In some embodiments, the pluripotent cell is an induced pluripotent stem cell. In some embodiments, the cell further comprises a homology-directed repair template polynucleotide. In some embodiments, the homology-directed repair template polynucleotide is a single-stranded DNA oligonucleotide (ssODN). In some embodiments, the CRISPR endonuclease is a Cas9. In some embodiments, the Cas9 is a catalytically dead Cas9 endonuclease, or a nickase Cas9 endonuclease. In some embodiments, the guide RNA is complementary to a sequence of an exon of the CSF1R gene. In some embodiments, the pluripotent stem cell comprises a mutation in a CSF1R gene. In some embodiments, the mutation in a CSF1R gene is associated with a leukodystrophy. In some embodiments, the pluripotent stem cell comprises two wildtype CSF1R genes. In some embodiments, the pluripotent stem cell comprises a repaired or replaced CSF1R gene. In some embodiments, the repaired or replaced CSF1R gene comprises a silent mutation.

In another aspect, the disclosure provides a method of preparing a cell therapy, the method comprising: editing the genome of an isolated cell to repair or replace a target gene; and incubating the isolated cell in a culture media comprising a differentiation factor, thereby generating an edited and differentiated cell. In some embodiments, the target gene comprises a disease-associated mutation. In some embodiments, the disease-associated mutation is a mutation associated with a leukodystrophy. In some embodiments, the leukodystrophy comprises an Adult-onset leukoencephalopathy (ALSP). In some embodiments, the isolated cell is an isolated human cell. In some embodiments, the isolated cell is an induced pluripotent stem cell. In some embodiments, the isolated cell was derived from a stem cell. In some embodiments, the isolated cell was derived from an iPSC. In some embodiments, the isolated cell was derived from a hematopoietic stem cell (HSC), a hematopoietic precursor cell (HPC), or a myeloid cell. In some embodiments, the edited and differentiated cell is a myeloid cell, a myeloid precursor cell, a myeloid progenitor cell, an erythro-myeloid precursor cell, an erythro-myeloid progenitor cell, a macrophage, a monocyte, a fetal macrophage, a hematopoietic stem cell-derived myeloid cell, or a yolk-sac-derived myeloid cell. In some embodiments, the edited and differentiated cell is an HPC or a microglial precursor cell. In some embodiments, the edited and differentiated cell is an iMGL. In some embodiments, the editing occurs before the incubating. In some embodiments, the editing occurs after the incubating. In some embodiments, the incubating comprises a first incubation period for differentiating an iPSC into an HPC and a second incubation period for differentiating the HPC. In some embodiments, the second incubation period is 1-28 days. In some embodiments, the cell culture media for the second incubation period comprises: IL-34, CSF-1, and TGFβ1; or IL-34, CSF-1, and a TGFβ mimetic. In some embodiments, the edited and differentiated cell can differentiate further in vivo. In some embodiments, the isolated cell was derived from a sample collected from a donor. In some embodiments, the sample comprises fibroblasts. In some embodiments, the sample comprises bone marrow. In some embodiments, the sample comprises blood or cerebrospinal fluid. In some embodiments, the method of preparing the cell therapy further comprises generating an iPSC from a cell of the sample before the editing or the incubating. In some embodiments, the editing comprises repairing a disease-associated mutation. In some embodiments, the disease-associated mutation causes reduced expression of the target gene, and wherein the edited and differentiated cell expresses at least 80% as much of the target gene as an otherwise identical control cell without the disease associated mutation or the editing. In some embodiments, the disease-associated mutation causes reduced activity of a polypeptide encoded by the target gene and wherein the edited and differentiated cell has at least 80% of the activity compared to an otherwise identical control cell without the disease associated mutation or the editing. In some embodiments, the editing comprises inserting a cDNA or fragment thereof within the target gene. In some embodiments, the disease-associated mutation comprises a mutation of a CSF1R gene. In some embodiments, the CSF1R gene is a human CSF1R gene. In some embodiments, the CSF1R mutation is within a nucleotide sequence encoding a kinase domain of a CSF1R polypeptide. In some embodiments, the mutant CSF1R gene encodes a CSF1R polypeptide comprises a point mutation with respect to a CSF1R polypeptide comprising SEQ ID NO: 1 or the fragment thereof. In some embodiments, the CSF1R mutation comprises a deletion mutation or an insertion mutation. In some embodiments, the editing comprises contacting the target gene with a TALEN, a zinc-finger endonuclease, a Base editor, a Prime editor, or a meganuclease. In some embodiments, the editing comprises contacting the target gene with a CRISPR endonuclease. In some embodiments, the CRISPR endonuclease comprises a Cas9 endonuclease. In some embodiments, the editing comprises introducing into the cell: a Cas9 polypeptide or a polynucleotide encoding the Cas9 polypeptide, a gRNA comprising a sequence of the CSF1R gene or a polynucleotide encoding the gRNA, and a polynucleotide comprising a CSF1R cDNA fragment, thereby replacing the CSF1R gene. In some embodiments, the gRNA comprises SEQ ID NO: 5. In some embodiments, the CSF1R cDNA fragment comprises exons 2-21 of the human CSF1R gene. In some embodiments, the polynucleotide comprising a CSF1R cDNA fragment further comprises a stop codon and a poly-A signal. In some embodiments, the CSF1R cDNA fragment comprises a silent mutation. In some embodiments, the CSF1R cDNA encodes a polypeptide having at least 90% sequence identity to a nucleotide sequence of SEQ ID NO: 1 or the fragment thereof. In some embodiments, the edited and differentiated cell comprises CSF1R proteins having at least 80% of the CSF1R tyrosine kinase activity of an otherwise identical control cell having exactly two copies of a wildtype CSF1R gene. In some embodiments, the method of preparing the cell therapy further comprises introducing into the cell: a Cas9 polypeptide or a polynucleotide encoding the Cas9 polypeptide, a gRNA complementary to a nucleotide sequence adjacent to the nucleotide sequence encoding the point mutation, and a homology-directed repair template polynucleotide comprising a wildtype CSF1R sequence at a position in the CSF1R gene corresponding to the position of the point mutation, thereby repairing the point mutation. In some embodiments, the homology-directed repair template polynucleotide is a single-stranded DNA oligonucleotide (ssODN). In some embodiments, the point mutation comprises a M875I mutation. In some embodiments, the gRNA comprises SEQ ID NO: 12. In some embodiments, the homology-directed repair template polynucleotide comprises SEQ ID NO: 7. In some embodiments, the point mutation comprises a L786S mutation. In some embodiments, the gRNA comprises SEQ ID NO: 13. In some embodiments, the homology-directed repair template polynucleotide comprises SEQ ID NO: 8. In some embodiments, the point mutation comprises a M785T mutation. In some embodiments, the gRNA comprises SEQ ID NO: 14. In some embodiments, the homology-directed repair template polynucleotide comprises SEQ ID NO: 9. In some embodiments, the point mutation comprises a N854K mutation. In some embodiments, the gRNA comprises SEQ ID NO: 15. In some embodiments, the homology-directed repair template polynucleotide comprises SEQ ID NO: 10. In some embodiments, the point mutation comprises a G598E mutation. In some embodiments, the gRNA comprises SEQ ID NO: 16. In some embodiments, the homology-directed repair template polynucleotide comprises SEQ ID NO: 11. In some embodiments, a proliferation rate of the edited and differentiated cell, is greater than a proliferation rate of an otherwise identical control iMGL, HPC or microglial precursor cell without the editing. In some embodiments, a transcriptional profile of microglia-related genes of the edited and differentiated iMGL more closely resembles a transcriptional profile of the microglia-related genes in a positive control iMGL or microglial precursor cell with exactly two native, wildtype CSF1R alleles compared to a transcriptomic profile of the microglia-related genes in an otherwise identical negative control iMGL comprising the defective CSF1R gene. In some embodiments, the method of preparing the cell therapy further comprises transplanting the edited and differentiated iMGL, HPC or microglial precursor cell into a brain of a subject. In some embodiments, wherein the transplanting: increases number or density of Iba1-expressing edited and differentiated microglia in the brain; increases number, density, or average size of excitatory synapses in the brain; increases PSD95 or NSE expression in the brain; decreases accumulation of secreted osteopontin (OPN) in the brain; decreases frequency of axonal spheroids immunoreactive for neurofilament SMI312 and LAMP1 in the brain; decreases number, density, or frequency of hydroxyapatite calcium crystals; decreases levels of Tau phosphorylated at Thr217 (pTau217) in the brain; decreases GFAP expression levels in the brain; or decreases MCP-1 expression levels in the brain, wherein at least 6 weeks has passed since the transplantation of the edited and differentiated iMGL, HPC, microglial precursor cell, or myeloid cell into the subject. In some embodiments, the transplanting is autologous and the donor is the subject. In some embodiments, the transplanting is allogeneic and the donor is not the subject. In some embodiments, the edited and differentiated iMGL, HPC or microglial precursor cell engrafts into and repopulates the brain of a subject with a leukodystrophy. In some embodiments, the edited and differentiated iMGL, HPC or microglial precursor cell is administered into the subject via a stereotactic, intraparenchymal, intracerebroventricular, intrathecal, subretinal, intraperitoneal, or intranasal injection. In some embodiments, the edited and differentiated iMGL, HPC or microglial precursor cell is administered into the subject via an intraperitoneal or intravenous injection.

Also provided herein is a method of editing a mutant CSF1R gene in an isolated myeloid cell comprising: repairing a point mutation in the mutant CSF1R gene, or inserting a CSF1R coding sequence 3′ of a FIRE in the mutant CSF1R gene, thereby repairing or replacing the mutant CSF1R gene in the myeloid cell. In some embodiments, the mutant CSF1R gene is associated with a leukodystrophy. In some embodiments, the leukodystrophy comprises Adult-onset leukoencephalopathy or BANDDOS. In some embodiments, the myeloid cell is an iPSC, HSC, or iMGL. In some embodiments, the myeloid cell was derived from an iPSC, HSC or HPC. In some embodiments, a sample comprising the isolated myeloid cell was collected from a donor. In some embodiments, the sample comprises fibroblasts or stem cells. In some embodiments, the point mutation is in a sequence encoding a kinase domain of a CSF1R polypeptide. In some embodiments, the editing comprises contacting the mutant CSF1R gene with a TALEN, a zinc-finger endonuclease, a Base editor, a Prime editor, or a meganuclease. In some embodiments, the editing comprises contacting the defective human CSF1R gene with a CRISPR endonuclease. In some embodiments, the CRISPR endonuclease comprises Cas9. In some embodiments, the repairing comprises repairing the point mutation by introducing into the cell: a Cas9 polypeptide or a polynucleotide encoding the Cas9 polypeptide, a gRNA complementary to a nucleotide sequence adjacent to the point mutation, and a homology-directed repair template polynucleotide comprising a wildtype CSF1R sequence at a position in the CSF1R gene corresponding to the position of the point mutation. In some embodiments, the homology directed repair template polynucleotide is a single-stranded DNA oligonucleotide. In some embodiments, the inserting comprises introducing into the cell: a Cas9 polypeptide or a polynucleotide encoding the Cas9 polypeptide, a gRNA comprising the nucleic acid sequence of SEQ ID NO: 5 or a polynucleotide encoding the gRNA, and a polynucleotide comprising a CSF1R coding sequence. In some embodiments, the polynucleotide further comprises a stop codon and a poly-A signal 3′ of the CSF1R coding sequence. In some embodiments, the polynucleotide further comprises a silent mutation. In some embodiments, the CSF1R coding sequence encodes a CSF1R polypeptide having at least 80% sequence identity to SEQ ID NO: 1 or a fragment thereof. In some embodiments, a proliferation rate of the isolated myeloid cell is greater than a proliferation rate of an otherwise identical control myeloid cell without the editing. In some embodiments, the isolated myeloid cell expresses CSF1R proteins having at least 80% of the CSF1R tyrosine kinase activity of an otherwise identical control cell having comprising two native wildtype CSF1R alleles. In some embodiments, the method of editing the mutant CSF1R gene in the isolated myeloid cell further comprises transplanting the myeloid cell into a subject. In some embodiments, the transplanting comprises a stereotactic, intraparenchymal, intracerebroventricular, intrathecal injection. In some embodiments, at least 6 weeks after the transplanting, the brain comprises: an increased number or density of Iba1-expressing microglia; an increased number, density, or average size of excitatory synapses; an increased PSD95 or NSE expression; a decreased accumulation of secreted osteopontin (OPN); a decreased frequency of axonal spheroids immunoreactive for neurofilament SMI312 and LAMP1; a decreased number, density, or frequency of hydroxyapatite calcium crystals; a decreased pTau217 level; a decreased GFAP expression level; or a decreased MCP-1 expression level. In some embodiments, the transplanting comprises autologous transplanting.

Another aspect of the disclosure includes a method of treating or preventing a leukodystrophy in a subject, comprising: administering to the subject any engineered cell described herein or a cell generation by any of the methods provided herein.

Provided herein is a method of treating or preventing a leukodystrophy in the subject comprising: obtaining a cell from the subject, wherein the cell was isolated from the subject or generated by culturing the cell isolated from the subject; producing an edited cell by repairing or replacing a defective gene in the cell from the subject; and transplanting the edited cell into the subject. In some embodiments, the subject was diagnosed with the leukodystrophy. In some embodiments, the subject is a risk of developing the leukodystrophy. In some embodiments, the leukodystrophy is Adult-Onset Leukoencephalopathy or BANDDOS. In some embodiments, the obtained cell is a stem cell, a myeloid cell, or a fibroblast. In some embodiments, the stem cell is a PSC or an iPSC. In some embodiments, the cell from the subject was derived from an iPSC. In some embodiments, the cell from the subject is an iMGL, an HPC or an MPC. In some embodiments, the edited cell differentiates into a microglial cell and engrafts into a microglial niche in a brain of the subject. In some embodiments, the mutant gene comprises a point mutation and the repairing comprises contacting the cell with Cas9, a guide RNA comprising a sequence of the mutant gene, and a single-stranded DNA oligonucleotide repair template. In some embodiments, the mutant gene comprises a CSF1R mutation. In some embodiments, the replacing comprises inserting a CSF1R coding sequence 3′ of the FIRE of the mutant CSF1R gene. In some embodiments, the transplanting comprises injecting the edited cell into a brain or a spinal cord of the subject. In some embodiments, according to the method of treating or preventing a leukodystrophy in the subject, the brain comprises: an increased number or density of Iba1-expressing microglia; an increased number, density, or average size of excitatory synapses; an increased PSD95 or NSE expression; a decreased accumulation of secreted osteopontin (OPN); a decreased frequency of axonal spheroids immunoreactive for neurofilament SMJ312 and LAMP1; a decreased number, density, or frequency of hydroxyapatite calcium crystals, a decreased pTau217 level; a decreased GFAP expression level; or a decreased MCP-1 expression level. In some embodiments, at least six weeks after the transplanting, the blood of the subject comprises decreased GFAP or pTau217. In some embodiments, at least 70% of the microglia in the brain of the subject after treatment are iMGLs or iMGLs derived from the engineered cell.

Also provided in the disclosure is a method of populating a microglial niche in the brain of a subject, comprising: obtaining cells with a repaired or replaced gene that corrects a disease-associated mutation, and administering the edited cells to the brain of the subject, wherein the obtained cells were isolated from the subject or generated by culturing cells that were isolated from the subject, and wherein the administered cells differentiate into microglia in vivo. In some embodiments, the subject was diagnosed with Adult-Onset Leukoencephalopathy. In some embodiments, the subject is at-risk of developing Adult-Onset Leukoencephalopathy. In some embodiments, the gene is CSF1R. In some embodiments, the subject harbors at least one allele comprising a mutant CSF1R gene. In some embodiments, the subject has a CSF1R haploinsufficiency. In some embodiments, the cells are iMGLs, HPCs, or microglial precursor cells. In some embodiments, the administering comprises: transplanting the cells into a brain or spinal cord of the subject.

In yet another aspect, the disclosure also provides a method of monitoring engraftment of an IMGL or microglial precursor cell in a brain comprising: transplanting the iMGLs or microglial precursor cells into the brain of a subject with a leukodystrophy, obtaining a blood sample from the subject, and measuring an amount of GFAP or pTau217 in the blood sample, wherein a decrease in the amount of GFAP or pTau217 in the blood sample indicates successful engraftment of the iMGLs or microglial precursor cells in the brain. In some embodiments, an amount of GFAP or pTau217 in the blood sample is decreased by at least 30% compared to an amount of GFAP or pTau217 in a blood sample from the subject prior to the transplanting. In some embodiments, the transplanted iMGLs or MPCs are not edited but instead comprise wildtype genes (e.g., CSF1R) from a healthy donor.

Also provided herein is a method of treating or preventing a leukodystrophy in a subject, the method comprising transplanting cells into the subject, wherein the cells differentiate into microglial cells and engraft into a microglial niche in a brain of the subject. In some embodiments, the transplanted cells are not edited but instead comprise wildtype genes (e.g., CSF1R) from a healthy donor. In some embodiments, the subject is at-risk of developing Adult-Onset Leukoencephalopathy. In some embodiments, the subject was diagnosed with the leukodystrophy. In some embodiments, the leukodystrophy is Adult-Onset Leukoencephalopathy. In some embodiments, the leukodystrophy is BANDDOS. In some embodiments, the transplanting comprises injecting the cells into a brain or spinal cord of the subject. In some embodiments, the cells are allogeneic cells. In some embodiments, the cells comprise iMGLs. In some embodiments, the cells comprise hematopoietic precursor cells or microglial precursor cells. In some embodiments, after the transplanting the brain comprises: an increased number or density of Iba1-expressing microglia; an increased number, density, or average size of excitatory synapses; an increased PSD95 or NSE expression; a decreased accumulation of secreted osteopontin (OPN); a decreased frequency of axonal spheroids immunoreactive for neurofilament SMI312 and LAMP1; a decreased number, density, or frequency of hydroxyapatite calcium crystals; a decreased pTau217 level; a decreased GFAP expression level; or a decreased MCP-1 expression level. In some embodiments, the subject comprises decreased GFAP or pTau217 expression levels in the brain or blood plasma after the transplanting. In some embodiments, at least 70% of the microglia in the brain of the subject after treatment are iMGLs or iMGLs derived from an engineered cell.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an illustration of the human CSF1R locus depicting integration of a hCSF1R coding sequence (CDS) and a poly-A signal into exon 2, which is located immediately downstream of the FIRE.

The black boxes represent introns.

FIG. 2 depicts a partial DNA chromatogram demonstrating successful targeting and CRISPR-mediated integration of CSF1R coding sequences downstream of the FIRE in ALSP6 iPSCs. LHA=Left Homology Ann. CSF1R SV40=CSF1R poly-A signal.

FIG. 3A maps the structural domains of CSF1R onto its exons and identifies point mutations associated with Adult-onset leukoencephalopathy. Most mutations are found within the tyrosine kinase domain (TKD) of CSF1R (Konno et. al., Neurology. 2018 Dec. 11; 91(24):1092-1104). Ig=immunoglobulin-like domain; UTR=untranslated region.

FIG. 3B depicts a schematic diagram of CSF1R gene and protein with mutations reported in BANDDOS. ALD—activation loop domain; Ig—immunoglobulin domain; RJD—regulatory juxtamembrane domain; TKD—tyrosine kinase domain.

FIG. 4 depicts partial DNA chromatograms demonstrating successful correction of the SNP by CRISPR-mediated insertion of ssODN in ALSP3 iPSCs heterozygous for the CSF1R-L786S point mutation.

FIG. 5 depicts partial DNA chromatograms demonstrating correction of the SNP by CRISPR-mediated insertion of ssODN in ALSP6 iPSCs heterozygous for the CSF1R-N854K point mutation.

FIG. 6 illustrates a scheme for disrupting the fms-intronic regulatory element (FIRE) of the mouse CSF1R gene showing the location of guide RNAs US1, DST1, and DST2 used to delete the FIRE in this mouse model. Black boxes=coding exons, white boxes=untranslated regions, lines connecting exons=intronic sequences, arrows=trophoblast/osteoclast promoter and macrophage transcription start sites.

FIG. 7A illustrates cryosections of wildtype (+/+) and homozygous FIRE (Δ/Δ) adult mouse brains stained with an antibody against the microglial cell marker IBA1. The images are representative of four mice per genotype.

FIG. 7B illustrates higher-magnification images of cryosections obtained from of wildtype (+/+) and homozygous FIRE (Δ/Δ) adult mouse brains stained with an antibody against IBA1, as shown in FIG. 7A. The boxed areas show IBA1⁺ perivascular macrophages in the Csf1rΔFIRE/αFIRE mouse brain. The images are representative of four mice per genotype.

FIG. 7C illustrates cryosections of wildtype (+/+) and homozygous FIRE (Δ/Δ) adult mouse brains stained with an antibody against the microglial cell marker P2RY12.

FIG. 7D presents flow cytometry data comparing the expression of CD45 and CD11b to identify populations of macrophages and microglia in single cell suspensions of myelin-depleted brains from Csf1rΔFIRE/ΔFIRE mice compared to heterozygous (+/Δ) and CSFR-WT (+/+) control mice.

FIG. 7E illustrates the quantification of flow cytometry data revealing the absence of microglia in a homozygous Csf1rΔFIRE/ΔFIRE mouse.

FIG. 8A illustrates a high magnification image of a typical axonal spheroid detected in an ALSP patient brain via immunohistochemical staining for pathological accumulation of neurofilament.

FIG. 8B illustrates immunohistochemical staining for pathological accumulation of neurofilament within axonal spheroids that develop in Csf1rΔFIRE/ΔFIRE mice brains, note the similar characteristic spheroid morphology observed in Csf1rΔFIRE/αFIRE mice and ALSP patient brains as shown in FIG. 8A.

FIG. 9A shows representative confocal images demonstrating the development of significant increases in brain calcification (osteopontin) and astrogliosis (glial fibrillary acidic protein: GFAP) in human Csf1rΔFIRE/ΔFIRE (hFIRE) mice at 8.5 months of age in comparison to littermates expressing wildtype human CSF1R (hCSF1R-WT).

FIG. 9B depicts quantified immunohistochemical image analysis of the number of osteopontin-positive (OPN+) cells in hFIRE mice in comparison to hCSF1R littermates at 8.5 months of age from imaging data as shown in FIG. 9A.

FIG. 9C depicts quantified immunohistochemical image analysis of the number of glial fibrillary acidic protein-expressing (GFAP+) (OPN+) cells in the thalamus of hFIRE mice in comparison to hCSF1R-WT littermates at 8.5 months of age from imaging data as shown in FIG. 9A.

FIG. 10 illustrates an experimental scheme whereby hCSF1R-WT and FIRE knockout (“FIRE KO”, Csf1rΔFIRE/ΔFIRE) mice were backcrossed with xenotransplantation-compatible “hCSF1 mice” that express a humanized CSF1 ligand and deletions in Rag2 and il2rg (M-CSF^{h}; Rag2^{tm1.1Flv}; II2rg^{tm1.1Flv}). This cross gives rise to “hFIRE” mice (Csf1rΔFIRE/ΔFIRE, M-CSF^{h}; Rag2^{tm1.1Flv}; IIrg^{tm1.1Fl}) that lack murine microglia and yet support human microglial xenotransplantation. Control littermates express humanized CSF1 but lack the FIRE deletion in CSF1R and therefore have mouse microglia. The mice were intracranially injected at 2 months of age with either human wildtype microglia-generating hematopoietic progenitor cells (hFIRE-HPC) or phosphate buffered saline solution (hFIRE-PBS). Control “hCSF1” mice that lack the FIRE deletion and therefore have normal numbers of murine microglia were also injected with phosphate buffered saline solution (control hCSF1-PBS). All animals are then sacrificed at 8.5 months of age, and their tissues subjected to biochemical analyses, including RNA sequencing (RNA-seq) and immunohistochemistry (IHC).

FIG. 11A and FIG. 111B show coronal brain sections from 8.5-month-old Csf1rΔFIRE/αFIRE mouse stained with antibodies specific to the microglial cell marker IBA1. FIG. 11A depicts the widespread engraftment of wildtype control human microglia 8 months after transplanting human iPSC-derived HPCs into the brain of Csf1rΔFIRE/ΔFIRE mice (hFIRE-HPC), indicated by immunohistochemical detection of IBA1 throughout a coronal section of the brain containing the hippocampus, cerebral cortex, thalamus, striatum, and midbrain.

FIG. 12A demonstrates that transplantation of human iMGLs prevents thalamic microbleeds. The images show Prussian Blue staining of microbleeds in thalamic brain sections obtained from hFIRE-HPC-injected mice, hFIRE-PBS-injected mice, and control hCSF1-PBS littermates at 2 months of age. Note the comparable lack of Prussian Blue staining in the thalamic sections of the hFIRE-HPC-injected mice and control hCSF1-PBS littermates.

FIG. 12B depicts the quantified population average of immunohistochemical analysis of Prussian Blue staining area per field-of-view (FOV) in thalamic brain sections of 2-month-old hFIRE-HPC, hFIRE-PBS, and control hCSF1-PBS littermates from images such as those as shown in FIG. 12A. Intracranial injection of microglia-generative human HPCs fully rescued the development of microbleeds in the hFIRE-HPC mice to levels comparable to that of control hCSF1-PBS littermates. Area μm²/FOV of Prussian Blue labeling of microbleeds in hCSF1^{WT/WT} mice (n=9) and hCSF1R^{ΔFIRE/ΔFIRE} transplanted with PBS (hFIRE-PBS; n=9) or transplanted human hematopoietic progenitor cells (hFIRE-HPC; n=9) analyzed by immunohistochemistry (1IC). The statistical significances were determined using one-way ANOVA, indicated in ****p<0.0001. Data are represented as mean value ±SEM.

FIG. 13A demonstrates a near complete reversal of brain calcification in hFIRE mice after intracranial injection of human iPSC-derived microglia-generating HPCs (hFIRE-HPC) in comparison to Csf1rΔFIRE/ΔFIRE littermates intracranially injected with PBS (hFIRE-PBS). Representative confocal images depict immunohistochemical analysis of IBA1 and OPN expression in brain sections obtained from hFIRE-HPC and hFIRE-PBS mice.

FIG. 13B depicts quantified immunohistochemical analysis of mean OPN expression level in brain sections obtained from hFIRE-HPC (n=9) and hFIRE-PBS (n=9) mice in confocal images as shown in FIG. 13A. Statistical significance was determined by unpaired t-test. ****p<0.0001

FIG. 13C depicts quantified immunohistochemical analysis of the percent area of the field-of-view (FOV) with OPN expression in brain sections obtained from hFIRE-HPC (n=9) and hFIRE-PBS (n=9) mice in confocal images as shown in FIG. 13A. Statistical significance was determined by unpaired t-test. ***p<0.001

FIG. 14A demonstrates that transplantation of human microglia-generating HPCs rescues brain calcification in hCSF1R^{ΔFIRE/ΔFIRE} mice. Representative confocal images show IBA1 expression and Risendronate-647 (RIS-647) levels in thalamic tissue sections obtained from hFIRE-HPC, hFIRE-PBS, and control hCSF1-PBS littermates. RIS-647 is a fluorescent bisphosphonate imaging reagent that binds strongly to hydroxyapatite calcium crystals.

FIG. 14B depicts the quantified population average of immunohistochemical analysis of IBA1 expression and RIS-647 signal in thalamic tissue sections obtained from hFIRE-HPC, hFIRE-PBS, and control hCSF1-PBS littermates, as shown in FIG. 13A(11A). RIS-647 is a fluorescent bisphosphonate imaging reagent that binds with high strongly to hydroxyapatite calcium crystals. Area um³/FOV of RIS-647 hCSF^{WT/WT} mice (control hCSF1-PBS; n=9) and hCSF1R^{ΔFIRE/ΔFIRE} transplanted with PBS (hFIRE-PBS; n=9) or human hematopoietic progenitor cells (hFIRE-HPC; n=9) analyzed by IHC. The statistical significances were determined using one-way ANOVA. **p<0.01. Data are represented as mean value f SEM.

FIG. 15A demonstrates that osteopontin (OPN)—an anti-inflammatory protein and regulator of biomineralization—surrounds RIS-647-labeled calcium deposits within the thalamus of PBS-injected hFIRE mice. Representative confocal images show histological labeling of osteopontin and RIS-647 in thalamic tissue sections obtained from a hFIRE-PBS mouse.

FIG. 15B demonstrates that transplantation of human HPCs prevented osteopontin accumulation and reactive astrogliosis in the thalamus of hCSF1R^{ΔFIRE/ΔFIRE} mice. Representative confocal images show immunohistological labeling of IBA1 expression (microglial cell marker), osteopontin (OPN) expression, and glial fibrillary acidic protein (GFAP; a cell marker of astrocytes) in thalamic brain sections obtained from hFIRE-HPC, hFIRE-PBS, and control hCSF1-PBS littermates.

FIG. 15C depicts the quantified population average summed intensity of OPN in thalamic brain sections obtained from hFIRE-HPC, hFIRE-PBS, and control hCSF1-PBS littermates in images as shown in FIG. 15B. Area um²/FOV of secreted proinflammatory cytokine OPN from hCSF1^{WT/WT} mice (n=9) and hCSF1R^{ΔFIRE/ΔFIRE} transplanted with PBS (hFIRE-PBS; n=9) or human hematopoietic progenitor cells (hFIRE-HPC; n=9) analyzed by IHC. The statistical significances were determined using one-way ANOVA, indicated in **p<0.01, ****p<0.001. Data are represented as mean value±SEM.

FIG. 15D depicts the quantified population average summed intensity of GFAP in thalamic brain sections obtained from hFIRE-HPC, hFIRE-PBS, and control hCSF1-PBS littermates in images as shown in FIG. 15B. Area um²/FOV of GFAP from hCSF1^{WT/WT} mice (n=9) and hCSF1RAF1R^{ΔFIRE/ΔFIRE} transplanted with PBS (hFIRE-PBS; n=9) or human hematopoietic progenitor cells (hFIRE-HPC; n=9) analyzed by IHC. The statistical significances were determined using one-way ANOVA, indicated in **p<0.01, ****p<0.0001. Data are represented as mean value ±SEM.

FIG. 16A demonstrates that transplantation of human HPCs prevented astrogliosis in hCSF1R^{ΔFIRE/ΔFIRE} mice as indicated by the quantified reduction of GFAP levels in soluble brain extracts obtained from hFIRE-HPC mice in comparison to hFIRE-PBS mice. hFIRE-HPC mice exhibited reduced GFAP levels comparable to control hCSF1-PBS littermates. Levels of GFAP in soluble brain samples from hCSF^{WT/WT} mice (n=9) and hCSF1R^{ΔFIRE/ΔFIRE} transplanted with PBS (hFIRE-PBS; n=9) or human hematopoietic progenitor cells (hFIRE-HPC; n=9) were analyzed by ELISA. The statistical significances were determined using one-way ANOVA indicated in **p<0.01, ***p<0.001. Data are represented as mean value±SEM.

FIG. 16B demonstrates that hFIRE-HPC mice exhibited reduced plasma GFAP in comparison to hFIRE-PBS mice. Levels of GFAP in plasma from hCSF1^{WT/WT} mice (n=9) and hCSF1R^{ΔFIRE/ΔFIRE} transplanted with PBS (hFIRE-PBS; n=9) or human hematopoietic progenitor cells (hFIRE-HPC; n=9) were analyzed by ELISA. The statistical significances were determined using one-way ANOVA indicated in **p<0.01, ***p<0.001. Data are represented as mean value±SEM.

FIG. 17 demonstrates that transplantation of human HPCs prevented increased levels of CCL2/MCP-1 in the brain of hCSF1R^{ΔFIRE/ΔFIRE} mice. The graph shows quantified population averages of MCP-1 levels in hFIRE-HPC, hFIRE-PBS, and control hCSF1-PBS littermates. Levels of CCL2/MCP-1 in soluble brain samples from hCSF1^{WT/WT} mice (n-9) and hCSF1R^{ΔFIRE/ΔFIRE} mice transplanted with PBS (hFIRE-PBS; n=9) or human hematopoietic progenitor cells (hFIRE-HPC; n=9) analyzed by ELISA. The statistical significances were determined using one-way ANOVA indicated in **p<0.01 and ***p<0.001. Data are represented as mean value±SEM.

FIG. 18A shows representative images of neurofilament SMI312-containing axonal spheroids in the thalamus hippocampus, cortex, and white matter (WM) within brains of hCSF1R^{ΔFIRE/ΔFIRE} mice.

FIG. 18B demonstrates that transplantation of HPCs prevents and reverses development of neurofilament SMI312-positive axonal spheroids. Quantified immunohistochemical data show that hFIRE-HPC brain tissue displayed minimal SMI312-positive spheroids that are markedly reduced in comparison to hFIRE-PBS mice but comparable to control hCSF1-PBS littermates. Number of SMI312+ spheroids per half brain of hCSF1^{WT/WT} mice (n=9) and hCSF1R^{ΔFIRE/ΔFIRE} transplanted with PBS (hFIRE-PBS; n=9) or human hematopoietic progenitor cells (hFIRE-HPC; n=9). The statistical significances were determined using one-way ANOVA, ***p<⁰0.001. Data are represented as mean value ±SEM.

FIG. 19A demonstrates that transplantation of human HPCs prevents and reversed formation of LAMP1-positive axonal spheroids. Representative confocal images show lysosomal markers LAMP1 and SMI312 in the fornix and white matter of brain sections obtained from hFIRE-HPC, hFIRE-PBS, and control hCSF1-PBS littermates and analyzed by immunohistochemistry.

FIG. 19B depicts quantified volumetric confocal image analysis of LAMP1-positive axonal spheroids in the hippocampal brain sections obtained from hFIRE-HPC, hFIRE-PBS, and control hCSF1-PBS littermates as shown in FIG. 19A.

FIG. 19C depicts quantified confocal image analysis of the number of LAMP1-positive axonal spheroids in the hippocampal brain sections obtained from hFIRE-HPC, hFIRE-PBS, and control hCSF1-PBS littermates as shown in FIG. 19A.

FIG. 19D depicts quantified volumetric confocal image analysis of LAMP1-positive axonal spheroids in the fornix of brain sections obtained from hFIRE-HPC, hFIRE-PBS, and control hCSF1-PBS littermates as shown in FIG. 19A.

FIG. 19E depicts quantified confocal image analysis of the number of LAMP1-positive axonal spheroids in the fornix of brain sections obtained from hFIRE-HPC, hFIRE-PBS, and control hCSF1-PBS littermates as shown in FIG. 19A.

FIG. 20 demonstrates that transplantation of human HPCs prevented loss of postsynaptic PSD95 protein in hCSF1R^{ΔFIRE/ΔFIRE} mice. Quantification of PSD-95 levels in soluble brain extracts from hCSF1^{WT/WT} mice (n=9) and hCSF1R^{ΔFIRE/ΔFIRE} mice transplanted with PBS (hFIRE-PBS; n=9) or human hematopoietic progenitor cells (hFIRE-HPC; n=9) analyzed by ELISA. The statistical significances were determined using one-way ANOVA, **p<0.01. Data are represented as mean value±SEM.

FIG. 21 demonstrates that transplantation of human HPCs prevented loss of neuron-specific enolase (NSE) in hFIRE mice. Quantification of NSE levels in soluble brain extracts from hCSF1^{WT/WT} mice (n=9) and hCSF1R^{ΔFIRE/ΔFIRE} mice transplanted with PBS (hFIRE-PBS; n=9) or human hematopoietic progenitor cells (hFIRE-HPC; n-9) analyzed by ELISA. The statistical significances were determined using one-way ANOVA, *p<0.1. Data are represented as mean value ±SEM.

FIG. 22A demonstrates that transplantation of human HPCs in 2-month-old hFIRE mice prevented an increase in Tau phosphorylated at threonine residue 217 (Tau-pT217) in soluble brain extracts. Levels of Tau-pT217 in soluble brain samples from hCSF1^{WT/WT} mice (n-9) and hCSF1R^{ΔFIRE/ΔFIRE} mice transplanted with PBS (hFIRE-PBS; n=9) or human hematopoietic progenitor cells (hFIRE-HPC; n=9) analyzed by ELISA. The statistical significances were determined using one-way ANOVA indicated as ****p<0.001. Data are represented as mean value±SEM. Samples were obtained from the same mice as in FIG. 20 and FIG. 21.

FIG. 22B demonstrates that transplantation of human HPCs in 2-month-old hFIRE mice exhibit a trend toward an increase in Tau phosphorylated at threonine residue 217 (Tau-pT217) levels in blood sera (p=0.366). Levels of Tau-pT217 in sera samples from hCSF^{WT/WT} mice (n=9) and hCSF1R^{ΔFIRE/ΔFIRE} mice transplanted with PBS (hFIRE-PBS; n=9) or human hematopoietic progenitor cells (hFIRE-HPC; n=9) analyzed by ELISA. No statistical significances were determined using one-way ANOVA indicated as ns. Data are represented as mean value±SEM. Samples were obtained from the same mice as in FIG. 20 and FIG. 21.

FIG. 23A depicts a process for isolating RNA from a brain tissue sample for bulk RNA-sequencing.

FIG. 23B is a heat map illustrating differences in the relative expression of human and mouse microglial genes in brain samples from the indicated mice, as measured by bulk RNA sequencing.

FIG. 24A demonstrates that transplantation of either HPCs after one day in microglial differentiation media (D1) or iMGLs (HPCs after 28 days in microglial differentiation media; D28) generated from an iPSC line obtained from a human donor (A75) prevented brain calcification and osteopontin accumulation in the thalamus of hFIRE mice. Representative confocal images showed microglia expressing IBA1 versus OPN and RIS-647 accumulation in thalamic brain sections obtained from hFIRE mice intracranially injected with either PBS or A75 HPCs (D1) or A75 iMGLs (D28) when mice were 2 months of age.

FIG. 24B demonstrates that iPSC-derived HPCs in microglial differentiation media after one day (D1) or iMGLs (HPCs after 28 days in microglial differentiation media; D28) derived from three different healthy human donors (A75, A76, and A77) prevented OPN accumulation and calcification in the thalamus of hFIRE mice. Representative confocal images show microglia expressing IBA1 versus OPN and RIS-647 accumulation in thalamic brain sections obtained from hFIRE mice which were intracranially injected with HPCs (D1) or iMGLs (D28).

FIG. 24C depicts the quantified population average summed intensity of OPN in thalamic brain sections obtained from hFIRE mice intracranially injected with one of three different human donor iPSC-derived HPCs or iMGLs in images as shown in FIG. 24A and FIG. 24B. Area (mm²/FOV) of Osteopontin (OPN) levels in hCSF1R^{ΔFIRE/ΔFIRE} (hFIRE) transplanted with PBS (hFIRE-PBS; n=3) or one of three different human donor iPSC-derived HPCs (cultured for one day in differentiation media; D1) or iMGLs (cultured for 28 days in differentiation media; D28) following analysis by immunohistochemistry (n=3 per cell type per cell line). The statistical significances were determined using one-way ANOVA, indicated in ****p<0.0001. Data are represented as mean value ±SEM.

FIG. 24D depicts the quantified population average summed intensity of RIS-647 in thalamic brain sections obtained from hFIRE mice intracranially injected with one of three different human donor iPSC-derived HPCs or iMGLs in images as shown in FIG. 13A and FIG. 13B. Volume (mm³/FOV) of Risedronate-647 (RIS-647) levels in hCSF1R^{ΔFIRE/ΔFIRE} (hFIRE) mice transplanted with PBS (hFIRE-PBS; n=3) or one of three different human donor iPSC-derived HPCs (cultured for one day in differentiation media; D1) or iMGLs (cultured for 28 days in differentiation media; D28) following analysis by immunohistochemistry (n=3 per cell type per cell line). HPCs and iMGLs were derived from three different human donor iPSC lines (A75, A76, and A77). The statistical significances were determined using one-way ANOVA, indicated in ****p<0.0001. Data are represented as mean value SEM.

FIG. 25 demonstrates increased proliferation of iMGLs from ALSP patient derived fibroblasts after CRISPR-mediated correction of an L786S point mutation. Line graph depicts a quantified 48-hour proliferation time course by measurement of culture confluency of iMGLs generated from human ALSP patient iPSCs expressing CSF1F-L786S (ALSP3-L786S) or CRISPR-mediated corrected of CSF1R (ALSP3-corrected). ALSP3-L786S and corrected patient iPSCs were differentiated in parallel for 14 days prior to assaying proliferation. The statistical significances were determined using two-way ANOVA (****p<0.0001).

FIG. 26A demonstrates that transplanted iMGLs comprising ALSP3-CSF1R-L786S exhibited low and localized levels of engraftment in the brains of 4-month-old hCSF1R^{ΔFIRE/ΔFIRE} mice after 6 weeks following intracranial injection into the hippocampus. Mice were 2-2.5 months of age at the time of intracranial transplantation. Representative confocal images showed immunohistochemical analysis of IBA1, Ku80, OPN, and RIS-647 in coronal sections containing the hippocampus obtained from 4-month-old hCSF1R^{ΔFIRE/ΔFIRE} mice after 6 weeks following intracranial injection of ALSP3-CSF1R-L786S iMGLs into the hippocampus. Iba1 immunoreactivity indicates the cytoplasm of microglia. Ku80 is a human-specific transcription factor with nuclear localization; Ku80 immunoreactivity is only present in the iMGLs. OPN immunoreactivity colocalizes with and surrounds the RIS-647 calcium deposits.

FIG. 26B demonstrates that transplanted iMGLs comprising a CRISPR-corrected CSF1R gene (ALSP3-corrected) fully engrafted throughout the brains of 4-month-old hCSF1R^{ΔFIRE/ΔFIRE} mice after 6 weeks following intracranial injection into the hippocampus. Mice were 2-2.5 months of age at the time of intracranial transplantation. Representative confocal images showed immunohistochemical analysis of IBA1, Ku80, OPN, and RTS-647 in coronal sections containing the hippocampus obtained from 4-month-old hCSF1R^{ΔFIRE/ΔFIRE} mice after 6 weeks following intracranial injection of either CRISPR-corrected ALSP3 iMGLs or ALSP3-CSF1R-L786S iMGLs (FIG. 26A) into the hippocampus. Iba1 immunoreactivity indicates the cytoplasm of microglia. Ku80 is a human-specific transcription factor with nuclear localization; Ku80 immunoreactivity is only present in the iMGLs. OPN immunoreactivity colocalizes with and surrounds the RIS-647 calcium deposits.

FIG. 27A demonstrates that transplantation of ALSP3-corrected iMGLs generated from an ALSP patient iPSC line reduced brain calcification and rescued disease-associated deficits in microglial proliferation in hCSF1R^{ΔFIRE/ΔFIRE} mice after 6 weeks following transplantation as compared to transplantation of ALSP3-L786S (uncorrected) iMGLs. Representative confocal images depict immunohistochemical analysis probing IBA1, Ku80, OPN, and RIS-647 levels in thalamic brain sections obtained from 4-month-old hCSF1R^{ΔFIRE/ΔFIRE} mice after 6 weeks following intracranial injection of either ALSP3-L786S iMGLs or ALSP3-corrected iMGLs. Mice were 2-2.5 months of age at the time of intracranial transplantation.

FIG. 27B depicts quantified summed volume per FOV (mm³/FOV) of RIS-647 levels in thalamic brain sections obtained from 4-month-old hCSF1R^{ΔFIRE/ΔFIRE} mice after 6 weeks following intracranial transplantation of either ALSP3-L786S iMGLs (n=3) or ALSP3-corrected iMGLs (n=3) analyzed by immunohistochemistry, as shown in FIG. 27A. The statistical significances were determined using t-test, indicated in *p<0.05. Data are represented as mean value±SEM. Bar graph demonstrates that transplantation of ALSP3-corrected iMGLs prevented and reversed excessive ALSP disease-associated brain calcification.

FIG. 27C depicts quantified summed area per FOV (μm²/FOV) of secreted proinflammatory cytokine OPN levels in thalamic brain sections obtained from 4-month-old hCSF1R^{ΔFIRE/ΔFIRE} mice after 6 weeks following intracranial transplantation of either ALSP3-L786S iMGLs (n=3) or ALSP3-corrected iMGLs (n=3) analyzed by immunohistochemistry, as shown in FIG. 23A. The statistical significances were determined using t-test, indicated in *p<0.05. Data are represented as mean value±SEM. Bar graph demonstrates that transplantation of ALSP3-corrected iMGLs prevented and reversed excessive ALSP disease-associated brain calcification.

FIG. 28A-28E demonstrate that CRISPR-corrected iMGLs surround remaining calcium deposits marked by RIS and OPN, suggesting that they may continue to clear this pathology likely via phagocytosis. FIG. 28A depicts representative confocal images of merged IBA, Ku80, OPN, and RIS-647 levels in a brain section obtained from a 4-month-old hCSF1R^{ΔFIRE/ΔFIRE} mouse after 6 weeks following intracranial transplantation of ALSP3-corrected iMGLs analyzed by immunohistochemistry. Dotted line indicates area of the higher magnification image inset (right). Representative confocal images of individual IBA1 (FIG. 28B), Ku80 (FIG. 28C), OPN (FIG. 28D), and RIS-647 (FIG. 28E) levels and distributions of the image inset in FIG. 28A.

FIG. 29A and FIG. 29B demonstrate that axonal spheroids are present in the hippocampal of Csf1rΔFIRE/ΔFIRE (hFIRE) mice as early as 2-months of age. The figures depict representative confocal images of LAMP1-immunoreactive axonal spheroids in the hippocampal sections obtained from 2-month-old hCSF1R^{WT/WT} mice (FIG. 29A) or 2-month-old Csf1rΔFIRE/ΔFIRE (hFIRE) mice (FIG. 29B). Arrows identify exemplary LAMP1-immunoreactive puncta indicative of axonal spheroids.

FIG. 29C and FIG. 29D demonstrate that axonal spheroids are present in the fimbria and fornix of Csf1rΔFIRE/ΔFIRE (hFIRE) mice as early as 2-months of age. The figures depict representative confocal images of LAMP1-immunoreactive axonal spheroids in the fimbria and fornix of brain sections obtained from 2-month-old hCSF1R^{WT/WT} mice (FIG. 29C) or 2-month-old Csf1rΔFIRE/ΔFIRE (hFIRE) mice (FIG. 29D). Arrows identify exemplary LAMP1-immunoreactive puncta indicative of axonal spheroids.

FIG. 29E and FIG. 29F demonstrate that axonal spheroids are present in the thalamus of Csf1rΔFIRE/ΔFIRE (hFIRE) mice as early as 2-months of age. The figures depict representative confocal images of aggregated OPN accumulation in thalamic sections obtained from 2-month-old hCSF1R^{WT/WT} mice (FIG. 29E) or 2-month-old Csf1rΔFIRE/ΔFIRE (hFIRE) mice (FIG. 29F) assessed by dense OPN-immunoreactive puncta. Arrows identify exemplary occurrences of OPN accumulation.

### DETAILED DESCRIPTION

### Definitions

The term “microglia” or “MG” as used herein, refers to resident innate immune cells of the CNS that play a role in synaptic plasticity, neurogenesis, homeostatic functions, and immune activity. In some cases, “microglia” may refer to naturally occurring microglia.

The term “pluripotent stem cell” or “PSC” as used herein, refers to cells that have the capacity to self-renew by dividing and to develop into the three primary germ cell layers of an early embryo, and therefore into all cells of an adult body.

The term “induced pluripotent stem cell” or “iPSC” as used herein, refers to a type of pluripotent stem cell that can be generated directly from a somatic cell.

The terms “microglial-like cell” and “iMGL” are used interchangeably to refer to “non-naturally occurring” microglial cells that resemble naturally occurring fetal and adult microglia. Microglial-like cells may be derived from stem cells, including pluripotent stem cells (PSCs), induced pluripotent stem cells (iPSCs), myeloid cells (e.g., myeloid precursor cells), and embryonic stem cells (ESCs). An iMGL may express at least 5 genes which are commonly expressed by naturally occurring microglia and display the same physiological and/or cellular functions but differ from the naturally occurring or non-genetically modified microglia by at least one aspect. Microglia-like cells may have gene expression signatures that are similar to but distinguishable from those of natural microglia cells. As a nonlimiting example, an iMGL may express a microglia-associated gene at a level that is increased or decreased by at least 10% of the expression level of the same gene in a naturally occurring microglia. In some instances, the microglia-like cells are ramified or actively phagocytic in nature. In some cases, iMGLs express AXL, P2RY12, TREM2, TMEM119, GPR84, P2RY6, CD45, and/or CD11b at a higher level than a non-immune cell. In some cases, iMGLs are capable of phagocytosis. In some cases, the iMGLs are capable of engrafting and proliferating throughout a brain of a subject. It shall be understood that the “microglia-like cells” or “iMGLs” of this disclosure can be genetically edited. In some cases, the iMGLs are generated by the artificial differentiation of a cell capable of differentiating into microglia-like cells (e.g., a hematopoietic progenitor cell, a myeloid cell, etc.) in vitro. In some cases, the iMGLs are generated by natural differentiation processes of a cell capable of differentiating into iMGLs upon transplantation into a subject.

The terms “hematopoietic progenitor cell” and “HPC” are used interchangeably to refer to a cell that can further differentiate into microglial progenitor cells (MPCs) and microglia-like cells (iMGLs). In cases where the HPC is engineered and/or genetically edited according to the methods of this disclosure, it shall be understood that the engineered HPC is a “non-naturally occurring” HPC and is different than natural HPCs and microglia by at least one aspect.

The terms “microglial progenitor cell” and “microglial precursor cell” (MPC) are used interchangeably to refer to a cell that can further differentiate into microglial-like cells in vitro. A microglial progenitor cell can be generated by contacting an HPC with a cell culture medium comprising differentiation factors of the disclosure. In cases where the microglia progenitor cell is engineered and/or genetically edited according to the methods of this disclosure, it shall be understood that the engineered microglial progenitor cell is a “non-naturally occurring” microglial progenitor cell and is different from natural microglial progenitors cells and microglia by at least one aspect. Microglial progenitor cells may include hematopoietic progenitor cells, erythromyeloid progenitor cells, primitive macrophages, and the like. Microglial progenitor cells may also be derived from pluripotent stem cells (PSCs), including induced pluripotent stem cells (iPSCs) and embryonic stem cells (ESCs).

The terms “synthetic allele” or “synthetic gene” as used herein may be used interchangeably and refer to a “non-naturally occurring” allele of a gene or the “non-naturally occurring” gene in which the genetic material of a cell is modified relative to a one or both alleles of the same gene in the genetic material of a reference cell from the same subject. In one nonlimiting example, the reference cell is a diploid germ-line cell from the same subject; in another nonlimiting example, the reference cell is a cell taken from the subject prior to a therapeutic intervention or non-naturally occurring manipulation according to this disclosure. Synthetic alleles or synthetic genes may be created through gene editing techniques that are known in the art, and may differ genetically (e.g., in their nucleic acid sequence) and/or epigenetically (e.g., in their DNA methylation status, histone acetylation status, chromatin structure, or in other aspects that do not materially alter the coding sequence) from naturally occurring, unmanipulated non-synthetic alleles of the same gene, or from the naturally occurring non-synthetic genes harbored by the unmanipulated reference cell from the subject. Synthetic alleles may be modified in their coding and/or non-coding sequence(s). In various aspects, synthetic alleles may include genetically modified (e.g., corrected) alleles of a CSF1R gene, or more preferably a human CSF1R gene (hCSF1R). It shall be understood that use of “synthetic allele” refers to one copy of the “synthetic gene,” and thus a modified cell harboring at least one synthetic gene refers to a modified cell harboring at least one synthetic allele.

The term “fragment” as used herein refers to a segment of a nucleic acid sequence consisting of DNA or RNA or may refer to a segment of an amino acid sequence for a protein. In some cases, the fragment may include a segment of the gene after the gene has been subjected to processes such as alternative splicing or intronic excision. As a nonlimiting example, a synthetic gene fragment may include the full-length protein coding sequence of the gene. In some cases, the synthetic gene fragment may include a full-length cDNA sequence or a portion of a cDNA sequence of the gene generated after reverse transcription.

“Autologous” as used herein, refers to cells or tissues derived from one subject, wherein the one subject may be both a donor and a recipient.

“Allogeneic” as used herein, refers to cells of the same species that are genetically different from the cells being compared.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993).

The terms “administer,” “introduce,” or “transplant” are generally used herein to encompass any technical means or methodologies of placing compositions of the disclosure (e.g., myeloid cells, microglia cells, HPCs) into the body of a subject in need thereof. For example, myeloid cells may be administered into the CNS by intravenous injection into the blood of a subject. In some cases, the method involves administering myeloid cells, microglia cells, or HPCs cells to the CNS of a subject. In some cases, the cells are directly injected into the CNS (e.g., brain, spinal cord) or the bloodstream of the subject. In some cases, administered MCs provided herein may differentiate into iMGLs in vitro, in vivo, or ex vivo. Alternatively, as a nonlimiting example, myeloid cells may be injected intracerebroventricularly into the brain parenchyma of a subject or can also be administered outside of the brain of a subject to generate macrophages and monocytes in peripheral tissues or blood of a subject. As a further nonlimiting example, compositions, formulations, and methods of the disclosure may be administered to the CNS of a subject as a liquid solution, as a semi-solid biomaterial, or as a solid biomaterial.

The terms “subject,” “host,” or “patient” are used interchangeably herein to refer to a vertebrate, preferably a mammal, and more preferably a human. Mammals include, but are not limited to, rodents (e.g., mice, rats, rabbits, etc.), simians, humans, non-human animals, non-human primates, primates, research animals (e.g., beagles, etc.), livestock animals, farm animals (e.g., pigs, horses, cows, etc.), sport animals, veterinary animals, and pets.

Generally, the terms “populate” or “repopulate” as used herein encompass the population growth of iMGLs, HPCs, or myeloid cells, following transplantation into a subject in need thereof. Both terms may be used interchangeably where appropriate. For example, where host endogenous microglia cells are not missing or depleted, the growth of introduced myeloid cells may be described in terms of “populating.” Conversely, where host endogenous microglia cells are missing or depleted, the growth of introduced iMGLs, HPCs, or myeloid cells may be described in terms of “repopulating.”

The terms “maturation,” “mature,” or “maturing” as used herein refer to the progression of the state of cellular differentiation for any of the cell types of this disclosure. “Maturation” is said to end when the cell becomes terminally differentiated into a mature iMGL or macrophage, which may be indicated by the presence of a cellular function, including but not limited to phagocytic activity. “Maturation” may be indicated by the presence, absence, or amount of cell marker expression profiles throughout the differentiation stages as a cell further advances from its original cell lineage as a pluripotent stem cell, induced pluripotent stem cell, or hematopoietic progenitor cell. As a nonlimiting example, an HPC may express low levels of CD11b compared to MPCs or iMGLs, and, further, an MPC may express lower levels of CD11b than a mature iMGL.

The term “purity” used herein references the percentage of cells of a target cell population measured to exist in a prepared sample relative to the total number of cells in the prepared sample. In some cases, “purity” is determined as the percentage of viable cells of the target cell population relative to the total number of viable cells in the prepared sample. As a nonlimiting example, a number of 70,000 iMGLs in a prepared sample containing 100,00 cells would determine that the population of iMGLs in the prepared sample is 70% pure.

The terms “treat,” “treating,” and “treatment” as used within this disclosure are meant to encompass any improvement in the signs or symptoms of a subject, including a reduction in the rate of disease progression, and also may encompass prophylactic or preventative or protective benefits. Improvement may cover any range of change from a measured numerical value indicative of the severity of signs and symptoms for a neurological disease. In some cases, the neurological disease is a leukodystrophy. For example, the improvement may be measured as a percentile change or a fold-change from a stated numerical value prior to treatment or a surgical procedure or as compared to an untreated subject or subject treated with a placebo. As a nonlimiting example, the improvement may be measured as a percentile change or a fold-change from a stated numerical value prior to transplantation of iMGLs, HPCs, or myeloid cells. As another example, the improvement be measured as a percentile change or a fold-change from a stated numerical value of a treated subject or subject receiving transplantation therapy as compared to another subject that did not receive treatment or transplantation therapy. In some cases, an improvement is measured by the absence or reduction of a pathological phenotype following treatment as compared within the same subject prior to treatment or as compared to a diseased or afflicted control subject. In some cases, an improvement is measured by the presence or increase of a non-pathological phenotype following treatment as compared within the same subject prior to treatment or as compared to a diseased or afflicted control subject. In some cases, an improvement is measured by a lesser or statistically insignificant percentile change or a fold-change from a stated numerical value of a treated subject or subject receiving transplantation therapy as compared to the same subject prior to treatment or as compared to another healthy unafflicted control subject.

The terms “therapeutically effective amount” generally refers to any amount or range of a therapeutic agent which elicits a therapeutic response in a subject with a neurological disease or disorder. In some cases, the therapeutic response can be the alleviation of one or more symptoms. In some cases, a therapeutic response may be a preventative treatment of a disease or a disorder. In some cases, the “effective amount or dose” may be that which is necessary or sufficient to produce a therapeutic response within the subject. Such an amount or dose may vary depending on the therapeutic agent used within the subject, as well as subject factors including, but not limited to age, weight, height, or general health of the subject in need of treatment.

The terms “formulated,” “prepared,” or “mixed” generally refer to a formulation containing a composition as described and provided within the disclosure which contains a technical element or limitation that renders said composition suitable for use in a subject in need thereof. Such formulations contain additional elements or limitations that further distinguish the composition over other compositions that lack such technical elements or limitations. In some nonlimiting examples, the formulation may comprise a culture medium used to support the proliferation, growth, maturation, or modulation of iMGLs, HPCs, or myeloid cells of the present disclosure.

The term “engineered cell” is used herein to refer to a “non-naturally occurring” cell. In some cases, a cell that has been genetically manipulated is an engineered cell. In some cases, a cell whose differentiation state has been manipulated in vitro can be an engineered cell.

The term “edited cell” is used to refer to a “non-naturally occurring” cell harboring one or more synthetic alleles or synthetic genes resultant of manipulation to alter the genetic material within the cell. An edited cell is structurally or functionally distinguishable in at least one aspect from the same type of cell naturally occurring in a subject that has not been subjected to a genetic modification.

The term “isolated cell” or “isolated source cell” refer to a cell that has been removed from a subject for further use. It shall be understood that an “isolated” cell must be isolated at least temporarily from the subject and that the “isolated” cell need not be permanently isolated from the subject. As a nonlimiting example, an isolated cell is obtained from a tissue sample acquired from a subject, then expanded in cell culturing conditions for a period of time sufficient for desired maturation, and eventually returned to the subject. Alternatively, the isolated cell may be obtained from a tissue sample acquired from the subject and manipulated ex vivo or in vitro for a sufficient amount of time to perform genetic modification of the isolated cell. The isolated cells described herein need not be returned to the subject. An isolated cell can be a pluripotent stem cell, a microglial-like cell, an induced pluripotent stem cell-derived microglial-like cell (iMGL), an HPC, a primitive macrophage, a yolk-sac-derived myeloid cell, or a myeloid precursor cell.

The terms “defective gene” and “mutant gene” are interchangeably used herein to describe a gene or an allele of a gene that is aberrantly expressed in an unmodified, naturally occurring cell. It shall be understood that a “defective gene” or “mutant gene” can result in changes in gene expression or encode a mutant protein with altered protein function (e.g., signaling) compared to a wildtype protein. In some cases, expression of the “defective gene” can constitute any deviation from normal expression levels of the gene, such as an increase or decrease in gene expression compared to expression of the wildtype version of the gene. As a nonlimiting example, a gene harboring a genetic mutation would be considered “defective” for generating less mRNA and/or protein in comparison to the same naturally occurring wildtype gene lacking the genetic mutation. A defective gene may be aberrantly expressed by at least a 20% increase, or a 20% decrease compared to the expression level of the same naturally occurring wildtype gene lacking the genetic mutation. In some cases, the “defective gene” encodes a protein with abnormal function or signaling compared to the protein encoded by the wildtype version of the gene. Abnormal function or signaling of a protein encoded by a “defective gene” can constitute a truncated protein lacking a signaling domain or a mutated protein exhibiting reduced or increased activity of a signaling domain of the defective protein compared to the wildtype protein. Signaling of a protein encoded by a defective gene may constitute at least a 20% increase or decrease of a stated numerical value as compared to the same mode of signaling of the same naturally occurring wildtype protein. As a nonlimiting example, a defective CSF1R gene may encode a CSF1R protein having an amino acid mutation that reduces CSF1R tyrosine receptor activity by at least 20% in comparison to the CSF1R tyrosine receptor activity of the wildtype CSF1R protein.

The term “point mutation” as used herein describes a mutation in a gene, mRNA transcript, or protein compared to the wildtype version of the gene, mRNA transcript, or protein. A point mutation may be a nucleotide substitution of one or very few nucleotides (<5 nucleotides) in a nucleic acid sequence of a gene. In some cases, a point mutation may be a single nucleotide polymorphism (SNP). In some cases, a point mutation may be an amino acid substitution of one amino acid in an amino acid sequence of a protein.

The term “silent mutation” as used herein describes a mutation in a nucleic acid sequence which encodes an amino acid at a position within amino acid sequence that is identical to the amino acid at the same position within a wildtype amino acid sequence. In some cases, the silent mutation is a point mutation or SNP in a nucleic acid sequence.

The term “replacing” as used herein describes the insertion of a synthetic gene or synthetic allele into the genome of a cell by gene editing methods. The synthetic gene can encode a protein with higher amino acid sequence similarity to the wildtype protein that a mutant gene present in the cell before editing. In some cases, “replacing” with a synthetic gene eliminates a pre-existing mutation in a cell with a defective gene to generate a DNA molecule encoding a protein having a higher sequence identity to the amino acid sequence of a wildtype version of the protein compared to the sequence identity of the amino acid sequence of the protein encoded by the defective gene, as measured by a percentile change. As a nonlimiting example, the “replacing” with a synthetic CSF1R gene may result in a CSF1R gene that encodes a protein having at least one less amino acid mutation than a defective CSF1R gene. As another nonlimiting example, the “replacing” with a synthetic CSF1R gene may result in a CSF1R gene that encodes a protein having higher sequence identity to the wildtype CSF1R protein as determined by aligning to the amino sequence of a wildtype CSF1R protein having the amino acid sequence of SEQ ID NO: 1 (UniProt ID No.: P07333-1).

The term “repairing” as used herein describes the use of gene editing methods to increase the sequence similarity of a nucleic acid sequence of a defective gene, or the amino acid sequence of the protein encoded by the defective gene, compared to the nucleic acid sequence, or amino acid sequence, of the wildtype version of the gene or protein, as determined by sequence alignment of the nucleic acid sequence or amino acid sequence of the wildtype version of the DNA molecule, mRNA transcript, or protein. In some cases, the “repairing” eliminates a pre-existing mutation in the DNA nucleic acid sequence of the defective gene locus to generate a DNA molecule having a DNA nucleic acid sequence with higher sequence identity to the wildtype gene As a nonlimiting example, a “repaired” CSF1R gene may result in expression of a CSF1R protein that possesses at least one less point mutation than the CSF1R protein encoded by a defective CSF1R gene.

The term “microglial niche” or “niche” are used interchangeably and refer to a localized compartment in the parenchyma of the central nervous system (e.g., brain, spinal cord, etc.) of a subject populated by microglia. Migrating microglia cells or iMGLs can infiltrate and populate the microglial niche. In some cases, a “niche” may be depleted of endogenous microglia, which would provide an uncrowded environment for transplanted MPCs, iMGLs, or MCs to proliferate within and, consequently, fill the niche.

The present disclosure relates to cells, such as human microglial-like cells (iMGLs), microglial progenitor cells (MPCs), hematopoietic progenitor cells (HPCs), and myeloid cells derived from pluripotent stem cells (PSCs) or induced pluripotent stem cells (iPSCs), that may include synthetic alleles of a human CSF1R gene. The present disclosure further relates to cells, such as human iMGLs, MPCs, HPCs, or MCs derived from PSCs or iPSCs, that express at least one canonical microglial marker. In various aspects, compositions including effective amounts of these cells may be administered to treat diseases associated with a mutation of a defective CSF1R gene.

The compositions, methods, and uses of this disclosure yield superior results for the generation of cell therapies for treating microglia-related and CSF1R gene-associated disorders, including leukodystrophies. The provided methods combine cell engineering with gene editing methods to create an edited and engineered microglial-like cell harboring repaired or replaced CSF1R genes to restore CSF1R expression or activity and to, consequently, enhance microglial survival, proliferation rates, microglia-intrinsic cellular functions (e.g., ADP-induced calcium transients, cytokine production and secretion, etc.), and other canonical cellular functions carried out by natural microglia in the brain of a subject (e.g., phagocytosis, migration, repopulating a microglia-depleted niche, etc.). The non-naturally occurring iMGLs of this disclosure closely resemble the core signature gene expression profiles of natural microglia and are able to more effectively engraft in and repopulate a microglia-depleted brain, as is observed in leukodystrophy patients, following transplantation into the brain of a subject, as compared to other cell types including bone marrow stem cells, macrophages, monocytes, etc. Furthermore, the in vitro differentiation method using PSC-derived or iPSC-derived iMGLs and MPCs provided herein provides ample opportunity for precise cloning and screening for CRISPR-mediated genetic editing of the iMGLs and MPCs, as well as expanding a relatively pure population of iMGLs or MPCs for transplantation into the subject, because the PSCs and iPSCs are self-renewing and can be maintained indefinitely to allow time for careful quality control assessment of the genetic editing. Transplanted bone marrow cells, by contrast, do not adopt gene expression profiles or cellular functions that closely resemble normal microglia. Hence, the compositions, methods, and uses of the iMGLs and MPCs described herein provide a novel therapeutic strategy for treating microglia-related disorders (e.g., leukodystrophies) by restoring microglia populations and brain homeostasis whilst also minimizing the likelihood of graft rejection, as the edited and differentiated iMGL and MPCs can be successfully derived from the patient intended to receive the cell therapy.

### Engineered Cells

The disclosure relates to compositions and methods of engineered cells that can be genetically edited to replace or repair a defective gene (e.g., defective CSF1R gene). The engineered cells described below can be differentiated from other cell types in vivo and transplanted into a subject according to the methods of the disclosure as a means for treating microglia-related genetic disorders, including leukodystrophies (e.g., Adult-Onset Leukoencephalopathy, BANNDOS). In some embodiments, the engineered cell is a microglia-like cell (iMGL), a microglial progenitor (MPC), a hematopoietic progenitor cell (HPC), or a myeloid cell (MC). In some instances, the engineered cells of this disclosure can be derived from a pluripotent stem cell obtained from a tissue sample of a donor or, alternatively, can be derived from an induced pluripotent stem cell (iPSC) reprogrammed from a donor tissue sample or tissue sample from a subject. In some instances, the engineered cell can be a PSC or iPSC, and the PSC or iPSC can be genetically edited according to the methods provided herein. In some instances, the engineered cells of the disclosure are preferably human cells. In some embodiments, the engineered cell is capable of differentiating into an IMGL, an HPC, an MPC, or a macrophage. In some embodiments, the engineered cell is capable of differentiating into a human iMGL, a human MPC, a human HPC, or a human macrophage. In some instances, the engineered cell comprises but does not express a mutant CSF1R gene. In some instances, the engineered cell comprises a wildtype CSF1R gene that has been repaired by editing out a mutation. In some instances, the engineered cell comprises a synthetic CSF1R, which is inserted into the genome of the engineered cell according to the methods of the disclosure. In some embodiments, the iMGL, HPC, MPC, or MCs of this disclosure harbor at least one defective gene prior to being edited. In some instances, the engineered cells described herein can harbor a defective gene, including but not limited to a defective CSF1R gene, and even more preferably a defective human CSF1R gene. In some embodiments, the defective CSFR1 gene comprises a mutation located 3′ of a fms-intronic response element (FIRE) or located within exons 2-21 of the defective CSF1R gene.

One aspect of the disclosure provides an engineered cell (e.g., human cell) comprising a Colony Stimulating Factor 1 Receptor (CSF1R) gene, wherein the CSF1R gene comprises a CSF1R coding sequence (CDS) encoding wild type CSF1R (e.g., human CSF1R) positioned 3′ of a fms-intronic regulatory element (FIRE) and 5′ of a mutation in the CSF1R gene, the CDS comprises a polyadenylation signal to prevent transcription of the mutation. In some embodiments, the CDS comprises a silent mutation effective for preventing enzyme-mediated DNA cleavage, e.g., Cas9 cleavage. In some embodiments, the engineered cell is a microglia-like cell (MGL). In some embodiments, the engineered cell is a microglial progenitor, a hematopoietic stem cell (HSC), or induced pluripotent stem cell. In some embodiments, the engineered cell is an iPSC-derived microglia cell.

One aspect of the disclosure provides an engineered cell (e.g., human cell) comprising a Colony Stimulating Factor 1 Receptor (CSF1R) gene, wherein the CSF1R gene comprises a CSF1R coding sequence (CDS) positioned 3′ of a fms-intronic regulatory element (FIRE) and 5′ of a mutation in the CSF1R gene. The CDS comprises a polyadenylation signal to prevent transcription of the mutation. The CDS encodes wild type CSF1R (e.g., human CSF1R), or at least a portion of wildtype CSF1R. In some embodiments, the CDS comprises a silent mutation effective for preventing enzyme-mediated DNA cleavage, e.g., Cas9 cleavage. In some embodiments, the engineered cell is a microglia-like cell (MGL). In some embodiments, the engineered cell is an induced pluripotent stem cell (iPSC)-derived microglia cell. In some embodiments, the engineered cell is a microglial progenitor cell or a hematopoietic stem cell (HSC). In some embodiments, the engineered cell is an induced pluripotent stem cell (iPSC). In some embodiments, the engineered cell is a microglial progenitor, a hematopoietic stem cell (HSC), or induced pluripotent stem cell. In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

One aspect of the disclosure provides an engineered cell (e.g., human cell) comprising a Colony Stimulating Factor 1 Receptor (CSF1R) gene, wherein the CSF1R gene comprises a CSF1R coding sequence (CDS) positioned 3′ of a fms-intronic regulatory element (FIRE) and 5′ of Exons 3-22 of the CSF1R gene. The CDS encodes wild type CSF1R (e.g., human CSF1R), or at least a portion of wildtype CSF1R. The CDS comprises a polyadenylation signal which blocks transcription of Exons 3-22. In some embodiments, the CDS comprises a silent mutation effective for preventing enzyme-mediated DNA cleavage, e.g., Cas9 cleavage. In some embodiments, the engineered cell is a microglia-like cell (MGL). In some embodiments, the engineered cell is an induced pluripotent stem cell (iPSC)-derived microglia cell. In some embodiments, the engineered cell is a microglial progenitor cell or a hematopoietic stem cell (HSC). In some embodiments, the engineered cell is an induced pluripotent stem cell (iPSC). In some embodiments, the engineered cell is a microglial progenitor, a hematopoietic stem cell (HSC), or induced pluripotent stem cell. In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

One aspect of the disclosure provides an engineered cell (e.g., human cell), the engineered cell being a microglial progenitor, hematopoietic stem cell, induced pluripotent stem cell (iPSC), or iPSC-derived microglial cell, comprising a Colony Stimulating Factor 1 Receptor (CSF1R) gene, wherein the CSF1R gene comprises a CSF1R coding sequence (CDS) inserted 3′ of a fms-intronic regulatory element (FIRE) and 5′ of a mutation in the CSF1R gene, wherein the CDS comprises a polyadenylation signal to prevent transcription of the mutation in the CSF1R gene. The CDS comprises at least a portion of a wild type human CSF1R sequence or a corrected sequence of human CSF1R, or the CDS encodes wild type CSF1R (e.g., human CSF1R) or at least a portion of wildtype CSF1R. In some embodiments, the CDS comprises a silent mutation effective for preventing enzyme-mediated DNA cleavage.

One aspect of the disclosure provides the use of a composition comprising an engineered cell (e.g., human cell) for a method of treating or preventing a leukodystrophy in a subject in need thereof, the method comprising a step of administering the composition to the subject, wherein the composition is effective for achieving a therapeutic concentration of microglia expressing non-mutated CSF1R in a brain of the subject, characterized in that the composition comprises an engineered cell comprising a Colony Stimulating Factor 1 Receptor (CSF1R) gene, wherein the CSF1R gene comprises a CSF1R coding sequence (CDS) positioned in Exon 2 and 3′ of a fms-intronic regulatory element (FIRE), the CDS comprises a polyadenylation signal to prevent transcription of Exons 3-22. In some embodiments, the CDS comprises a silent mutation effective for preventing enzyme-mediated DNA cleavage by Cas9. In some embodiments, the engineered cell is a microglia-like cell (MGL). In some embodiments, the engineered cell is an induced pluripotent stem cell (iPSC)-derived microglia cell. In some embodiments, the engineered cell is a microglial progenitor cell or a hematopoietic stem cell (HSC). In some embodiments, the engineered cell is an induced pluripotent stem cell (iPSC). In some embodiments, the engineered cell is a microglial progenitor, a hematopoietic stem cell (HSC), or induced pluripotent stem cell. In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

One aspect of the disclosure provides an engineered induced pluripotent stem cell (iPSC) comprising a mutant Colony Stimulating Factor 1 Receptor (CSF1R) gene comprising a point mutation, a Cas9 polypeptide or a polynucleotide encoding the Cas9 polypeptide, a gRNA complementary to a nucleotide sequence adjacent to the point mutation, and either (i) a homology-directed repair template polynucleotide comprising a wildtype CSF1R sequence at a position in the CSF1R gene corresponding to the position of the point mutation or (ii) a polynucleotide comprising a CSF1R coding sequence. The Cas9 polypeptide repairs or replaces the point mutation of the mutant CSF1R and thus generates a wildtype CSF1R.

### Engineered Microglia-Like Cells, Microglial Progenitor Cells, Hematopoietic Progenitor Cells, and Myeloid Cells

In various aspects, the iMGLs, microglial progenitor cells (MPCs), hematopoietic progenitor cells, or myeloid cells (MCs) of the disclosure may be generated from PSCs or iPSCs using processes known in the art. In some aspects, the iMGLs, MPCs, HPCs, or myeloid cells of the disclosure may be generated from iPSCs or PSCs using processes known in the art. In some aspects, the hematopoietic progenitor cells may include erythromyeloid progenitor cells, or primitive macrophages. In some aspects, the iMGLs may be derived from HPCs or MPCs.

### Myeloid Cells

In some cases, the engineered cell of this disclosure may be a myeloid cell (MC). The MCs (or cells of a myeloid lineage) provided herein may include macrophages, monocytes, bone marrow cells, blood cells, yolk sac cells, fetal brain macrophages, fetal liver macrophages, microglia-like cell (iMGL), or any other myeloid-lineage cells. In some cases, MCs may be one or more lineages of blood cells arising from multipotent hematopoietic stem cells (HSCs) that are involved in dendritic cell formation.

In some cases, iMGLs may be derived from yolk-stem cells (e.g., fetal brain) and may possess a gene expression profile that more closely resembles microglia cells than that seen for iMGLs derived from hematopoietic stem cells (e.g., from blood or bone marrow) or iMGLs derived from a mixed origin (e.g., fetal liver). In some cases, the myeloid cells may be derived from embryonic or extraembryonic tissue. In some cases, the myeloid cells may be derived from postnatal tissue.

During hematopoiesis, a common myeloid progenitor cell arises along with a common lymphoid progenitor cell that differentiates into the lymphoid cell lineage comprising of T cells, B cells, and natural killer (NK) cells. In some cases, myeloid progenitor cells can differentiate into multiple cell types and lines including monocytes, macrophages, neutrophils, basophils, eosinophils, erythrocytes, megakaryocytes which produce thrombocytes, and mast cells. Differentiation and proliferation of myeloid progenitor-derived cells can be influenced by a variety of growth factors and cytokines. In some cases, specific combinations of growth factors and cytokines may help control the differentiation pathway potential for myeloid progenitor cells.

In general, this disclosure provides MCs that are myeloid progenitor cells capable of differentiating into microglia-like cells in vitro, ex vivo, or in vivo. As described herein, in some aspects, the MCs may be myeloid progenitor cells that can differentiate into iMGLs after administration to a subject in need thereof.

In some cases, the MC may possess a gene expression profile similar to those of normal, healthy, or natural microglia cells (e.g., the microglia sensome/phenotype). In some cases, MCs or MC-derived iMGLs may express genes or proteins that are specific to microglia. For example, MCs or MC-derived iMGLs may express the following microglia specific gene biomarkers: Tmem119, P2ry12, Olfml3, Sall1, Gpr34, Gpr56, and Gpr84, or any subset thereof. In another example, MCs may express the following microglia specific protein biomarkers: transmembrane protein 119 (TMEM119), P2Y purinoceptor 12 (P2RY12), olfactomedin-like protein 3 (OLFML3), Sal-like protein 1 (SALL1), G protein-coupled receptor 34 (GPR34), G protein-coupled receptor 56 (GPR56), and G protein-coupled receptor 84 (GPR84), or any subset thereof. In some cases, MCs or MC-derived iMGLs may express one or more additional gene biomarkers including, but not limited to, Cd45, Cd11b, Iba1, Clec12a, Ms4a7, Lilra5, Klra2, or any combination thereof. In some cases, MCs or MC-derived iMGLs may express one or more additional protein biomarkers including, but not limited to, cluster of differentiation 45 (CD45), cluster of differentiation 11B (CD11B), ionized calcium binding adapter molecule 1 (IBA1), C-type lectin domain family 12 member A (CLEC12A), membrane-spanning 4-domains subfamily A (MS4A7), leukocyte immunoglobulin-like receptor subfamily A member 5 (LILRA5), killer cell lectin-like receptor 2 (KLRA2), or any combination thereof. In some cases, the cells may express genes or proteins that are more specific to microglia cells than those expressed in microglia cells derived from HSCs. In some cases, the MCs or MC-derived iMGLs introduced into a subject may be conditioned by the CNS to express microglia-specific genes or proteins.

### Macrophages and Monocytes

Macrophages are myeloid-derived cells of the immune system and are found in tissues including, but not limited to, bone marrow and blood. Macrophages are ontogenically different from native microglia in that macrophages are derived from the hematopoietic stem cell lineage, while native microglia are derived from the embryonic yolk-sac lineage that migrate into the developing central nervous system and then self-renew throughout the lifespan of an organism (e.g., mouse, human, etc.). With respect to the objectives of the disclosure, this ontogenic distinction makes HSC-derived macrophages more suitable for cell therapeutic strategies of disorders or conditions involving the depletion of blood and bone marrow immune cells because, in contrast to the iMGLs provided herein, HSC-derived macrophages are not capable of fully adopting the signature gene expression profiles of microglia, continue to exhibit functional differences from brain-resident microglia many months after brain engraftment, and do not play the same role in disease (Bennett et al., A combination of ontogeny and CNS environment establishes microglial identity, Neuron 2018 Jun 27; 98(6): 1170-1183.e8). While HSC-derived macrophages are capable of engrafting in the brain of a subject and mimicking some cellular functions of microglia (e.g., phagocytosis, etc.), they do not express Sall1 and exhibit lower expression of P2RY12, Tmem119, Fcrls, Hexb, and Olfml3 in comparison to native microglia. In some embodiments, the macrophages of this disclosure are isolated from a donor and further edited (i.e., genetically edited) to correct or repair a defective gene in the isolated MC-derived macrophage. In some embodiments, the macrophages of the disclosure can be edited to express the synthetic CSF1R gene described herein. In some embodiments, the MC-derived macrophages of the disclosure may be edited (i.e., genetically edited) to repair a defective CSF1R gene in

Monocytes are a cell type related to macrophages and have a gene expression profile distinct but non-overlapping from that of the iMGLs and HPCs of the disclosure. For example, monocytes may express TMEM119, CD45, c-kit, Ly6c, NK1.1, CD3, B220, Jl7ra, Siglecf, Ly6g, and CCR2. The edited myeloid cells described herein can differentiate into monocytes and macrophages, thus repopulating the blood with CSF1R-corrected macrophages.

Hematopoietic progenitor cells Hematopoietic progenitor cells (HPCs) are “primitive” stem cells that are derived from the yolk sac lineage during early embryonic development. These HPCs then begin to differentiate into erythromyeloid progenitors and microglial progenitor cells (MPCs) which migrate into the brain and then differentiate into microglia. It shall be understood that the HPC described herein is capable of further maturing into an MPC or iMGL capable of performing all microglial cellular functions as described below. In some cases, the engineered cell of this disclosure can be a hematopoietic progenitor cell (HPC). In some embodiments, the HPC can differentiate into an iMGL or MPC but has higher CD43 expression than the iMGL described below. The HPC can be of a commercial HPC cell line, derived from a commercial cell line capable of differentiating into an HPC, or can be differentiated from a PSC or iPSC acquired from a subject or according to the methods described below or in any of U.S. patent application Ser. No. 16/489,338, U.S. patent application Ser. No. 14/986,224, Bennett et al. New tools for studying microglia in the mouse and human CNS, PNAS (2016), Abud et al., iPSC-Derived Human Microglia-like Cells to Study Neurological Diseases Neuron (2017), or McQuade et al., Development and validation of a simplified method to generate human microglia from pluripotent stem cells Molecular Neurodegeneration (2018), which are hereby incorporated by reference.

In some instances, the engineered cell may be a hematopoietic progenitor cell that was differentiated from either a PSC or an iPSC. In some cases, the HPC can be a CD43-expressing (CD43+), CD31-expressing (CD31+), or CD34-expressing (CD34+) HPC. In some cases, the HPC expresses CD45 (CD45+). The HPC may express another marker known to identify HPCs. In some cases, the engineered HPC expresses CD45. In some cases, the engineered HPC lacks expression of CX3CR1. In some cases, the engineered HPC has a CD45+/CX3CR1− or CD45+/CX3CR1+ expression profile. In some cases, the HPC expresses CD34 and is therefore distinguishable from PSCs, which lack CD34 and CD31 expression.

In general, this disclosure provides engineered HPCs capable of differentiating into microglial progenitor cells or microglia-like cells in vitro, ex vivo, or in vivo. As described herein, in some aspects, the HPCs may be engineered and then continues to differentiate into MPCs and iMGLs in vivo after administration to a subject in need thereof. The engineered HPC described herein may express cell markers that facilitate its detection or demarcate its stage of maturity as it differentiates into the MPC or iMGL described below (e.g., CD43, CD34, etc.).

### Microglial Progenitor Cells

In some cases, the engineered cell of this disclosure may be a microglial progenitor cell (MPC). In some embodiments, the engineered MPC may be derived from a PSC. In some embodiments, the engineered MPC may be derived from an iPSC. In some embodiments, the engineered MPC is derived from an HPC. The microglial progenitor cells described herein are capable of further maturing into an iMGL capable of performing all microglial cellular functions as described below. In some cases, for example, the MPC may be identified and distinguished from an HPC by having higher average CD11b expression than HPCs.

The engineered MPC described herein may express cell markers that facilitate its detection or demarcate its stage of differentiation as it matures into an iMGL with the canonical microglia transcriptomic signature described below. As one nonlimiting example, the engineered MPC may express low levels of CD11b (CD11b^{lo}) compared to a mature engineered iMGL that has been differentiated for at least about 5 days in vitro (i.e., exposed to the cell culture media with differentiation factors described below). The engineered MPC can gradually increase CD11b expression throughout the maturation process. In some cases, the engineered MPC expresses CD45. In some cases, the engineered MPC lacks expression of CX3CR1. In some cases, the engineered MPC has a CD45+/CX3CR1− or CD45+/CX3CR1+ expression profile. In some cases, the engineered MPC of this disclosure can be exposed to differentiation factors IL-34, CSF1R, and/or TGFβ types as described below for at least about one day and no more than about 5 days in vitro to be distinguishable from the engineered iMGL described above. In some cases, the engineered MPC expresses higher CD43 expression levels than the engineered iMGL of this disclosure. In some cases, for example, the MPC may be identified and distinguished from a PSC or iPSC stem cell by the expression of CD1 b. Additionally, the MPC may be identified and distinguished from an iMGL by a relatively lower CD11b expression compared to that of a mature iMGL.

It shall be understood that the MPCs described herein are capable of further maturing into an iMGL capable of performing all microglial cellular functions as described above. It shall also be understood that an MPC can be generated by contacting the HPC with the culture media containing differentiation factors IL-34. CSF-1, and TGFβ, as described below, for an incubation period of at least one day. AN MPC is an intermediary cell type between the HPC and the iMGL, and has not yet developed the core microglial gene expression signature of canonical microglial markers described below. In some cases, the MPC can be exposed to the differentiation factors described herein for no more than about 5 days to be considered an MPC that has not yet differentiated into an iMGL.

### Microglia-Like Cells

The engineered iMGLs may express any factor or any combination of factors that a typical canonical microglial cell expresses in a mature, terminally differentiated state. In some instances, the iMGLs may express gene expression profiles of an intermediary stage of iMGL maturation, including but not limited to increasing expression levels of CD11b, P2RY12, TREM2, etc. following more time after in vitro differentiation according to the methods of this disclosure. In some aspects, the iMGL expresses c-kit−/CD45. In some aspects, the iMGLs expresses either: (1) CD45+/CX3CR1—or (2) CD45+/CX3CR1+. In some aspects, the iMGL expresses CD43, CD235a, CD41, or any combination thereof. In some aspects, the iMGL expresses at least two of CD43, CD235a, and CD41. In some aspects, the iMGL expresses CD43, CD235a, and CD41. In some cases, the iMGLs express a lower level of CD34 expression in comparison to HPCs.

In some aspects, the iMGL expresses cell markers of committed iMGL lineage. In some aspects, the iMGL expresses the transcription factor PU.1 and/or the microglia-enriched proteins P2RY12 or TREM2. In some embodiments, the iMGL further expresses TMEM119. In some embodiments, the iMGL further expresses AXL, STAB1, P2RY6, CCR6, GPR84, or any combination thereof. In some embodiments, the iMGL further expresses AXL, STAB1, P2RY6, CCR6, and GPR84. In some embodiments, the iMGL further expresses P2RY13 or OLFML3. In some embodiments, the iMGL further expresses P2RY13 and OLFML3. In some embodiments, the iMGL further expresses CD9. In some embodiments, the iMGL further expresses neprilysin (NEP). In some embodiments, the iMGL expresses of any one, or any combination of two or more, of the following genes: RUNX1, SPI1, CSF1FR, CX3CR1, TGFBR1, RSG10, GAS6, MERTK, PSEN2, PROS1, P2RY12, P2RY13, GPR34, CIQ, CR3, CABLES1, BHLHE41, TREM2, TYROBP, ITGAM, APOE, SLCO2B1, SLC7A8, PPARD, TMEM119, GPR56, C9orf72, GRN, LRRK2, TARDBP, and CRYBB1.

The engineered iMGLs described herein may comprise at least one canonical microglial marker, wherein the canonical microglial marker comprises: P2RY12, LAPTM5, CX3CR1, CSF1R, C1QC, C1QA, C1QB, SELPLG, P2RY13, GPR34, TYROBP, TREM2, PLD4, CD53, CTSS, TMEM119, ITGAM, LY86, SPI1, SASH3, FYB, FCGR1, CD86, BTK, IRF8, TLR7, HCK, NCKAP1I, BIN2, FCERIG, HCLS1, RASAL3, DOCK2, CSF3R, PTAFR, PLCB2, ADORA3, AIF1, AXL, STAB1, P2RY6, CCR6, GPR84, TREM2, CD45, or any combination thereof. The engineered iMGLs described herein may comprise at least two canonical microglial markers, wherein the canonical microglial marker comprises: P2RY12, LAPTM5, CX3CR1, CSF1R, C1QC, C1QA, C1QB, SELPLG, P2RY13, GPR34, TYROBP, TREM2, PLD4, CD53, CTSS, TMEM119, ITGAM, LY86, SPI1, SASH3, FYB, FCGR1, CD86, BTK, IRF8, TLR7, HCK, NCKAP1I, BIN2, FCER1G, HCLS1, RASAL3, DOCK2, CSF3R, PTAFR, PLCB2, ADORA3, AIF1, AXL, STAB1, P2RY6, CCR6, GPR84, TREM2, CD45, or any combination thereof. The engineered iMGLs described herein may comprise at least three canonical microglial marker, wherein the canonical microglial marker comprises: P2RY12, LAPTM5, CX3CR1, CSF1R, C1QC, C1QA, C1QB, SELPLG, P2RY13, GPR34, TYROBP, TREM2, PLD4, CD53, CTSS, TMEM119, ITGAM, LY86, SPI1, SASH3, FYB, FCGR1, CD86, BTK, IRF8, TLR7, HCK, NCKAP1I, BIN2, FCERIG, HCLS1, RASAL3, DOCK2, CSF3R, PTAFR, PLCB2, ADORA3, AIF, AXL, STAB1, P2RY6, CCR6, GPR84, TREM2, CD45, or any combination thereof.

In various aspects, the iMGLs or microglial progenitor cells of the disclosure may comprise cells that express at least one canonical microglial marker. In some aspects, the cells may express one or more microglial markers selected from: RUNX1, SPI1, CSF1R, CX3CR1, TGFBR1, RSG1O, GAS6, MERTK, PSEN2, PROS1, P2RY12, P2RY13, GPR34, C1Q, CR3, CABLES1, BHL-HE41, TREM2, TYROBP, ITGAM, APOE, SLCO2B1, SLC7A8, PPARD, TMEM119, GPR56, C9orf72, GRN, LRRK2, TARDBP, CRYBB1, and combinations thereof.

In some aspects, TRIM14, CABLES1, MMP2, SIGLEC 11 and SIGLEC12, MITF, and/or SLC2A5 mRNA and/or protein expression may be enriched in the produced iMGLs. In some aspects, COMT, EGR2, EGR3, and/or FFAR2 mRNA and/or protein expression is enriched in the produced iMGLs.

In some aspects, iMGLs may be provided that express a specific gene profile. Any of the iMGLs described herein may comprise a gene expression profile similar to canonical microglia cells. In some aspects, any of the compositions of iMGLs described herein comprise expression of any of the following genes: RUNX1, PU.1, CSF1R, CX3CR1, TGFBR1, RSG10, GAS6, PROS1, P2RY12, GPR34, C1Q, CR3, CABLES1, BHLHE41, TREM2, ITAM, APOE, SLCO2B1, SLC7A8, PPARD, C9orf72, GRN, LRRK2, TARDBP, and CRYBB1. Any of the iMGLs disclosed herein may comprise expression of any of these genes in any combination: RUNX1, SPI1, CSF1R, CX3CR1, TGFBR1, RSG10, GAS6, MERTK, PSEN2, PROS1, P2RY12, P2RY13, GPR34, C1Q, CR3, CABLES1, BHLHE41, TREM2, TYROBP, ITGAM, APOE, SLCO2B1, SLC7A8, PPARD, TMEM119, GPR56, C9orf72, GRN, LRRK2, TARDBP, and CRYBB1.

In some aspects, in any of the compositions of iMGLs described herein TREM2 and P2RY12 may be co-expressed. In some aspects, any of the compositions of iMGLs described herein may not express any one or more of the genes KLF2, TREM1, MPT, ITGAL, and ADGRE5.

In some cases, the iMGLs of the disclosure are exposed to the cell culture medium comprising differentiation factors described below for a period of at least about 5 days. In some cases, the iMGLs of the disclosure are incubated in the cell culture medium comprising differentiation factors described below for a period of at least about 7 to 14 days or any other incubation period described below.

Comparative Gene Expression of iMGLs vs Natural Microglia

The iMGLs described herein express microglial genes in a manner different than the expression levels of the same genes of natural fetal microglia or adult microglia. In some embodiments, the iMGLs express a higher level of any one of AXL, STAB1, P2RY6, CCR6, or GPR84 compared to natural adult microglia. In some embodiments, the iMGLs express a higher level of any two of AXL, P2RY6, CCR6, or GPR84 compared to natural adult microglia. In some embodiments, the iMGLs express a higher level of any one of AXL, P2RY6, CCR6, and GPR84 compared to adult microglia. In some embodiments, the iMGLs express lower levels of at least one of CTSL, CTSD, or NPL compared to natural fetal microglia or adult microglia. In some embodiments, the iMGLs express lower levels of at least two of CTSL, CTSD, or NPL compared to natural fetal microglia or adult microglia. In some embodiments, the iMGLs express lower levels of CTSL, CTSD, and NPL compared to natural fetal microglia or adult microglia. In some embodiments, the engineered iMGL may have a transcriptional profile of microglia-related genes of the edited and differentiated iMGL more closely resembling a transcriptional profile of the microglia-related genes in a positive control iMGL or microglial precursor cell with exactly two native, wildtype CSF1R alleles compared to a transcriptomic profile of the microglia-related genes in an otherwise identical negative control iMGL comprising the defective CSF1R gene.

Engineered iMGL Functions

It shall be understood that the engineered iMGL of this disclosure may be capable of cellular functions carried out by natural microglia. In some embodiments, the engineered iMGL may be capable of engrafting into a microglia-depleted niche in the brain of a subject. In some embodiments, the engineered iMGL may be capable of secreting pro-inflammatory markers. In some embodiments, the engineered iMGL may be capable of ramification. In some embodiments, the engineered iMGL may be capable of endocytosing amyloid-0 oligomers, neurofilaments (e.g., Tau-expressing neurofilaments, etc.), or hydroxyapatite calcium crystals (e.g., risedronate-reactive calcium crystals, etc.). In some embodiments, the engineered iMGL may be capable of producing calcium transients. In some embodiments, the engineered iMGL may be capable of producing calcium transients upon stimulation with adenosine diphosphate (ADP). In some embodiments, the engineered iMGL may be capable of migrating in response to chemical stimulation, including but not limited to ADP chemical stimulation. In some embodiments, the engineered iMGL may be capable of proliferating in vivo or in vitro. In some embodiments, the engineered iMGL may be capable of endocytosing human synaptosomes. In some embodiments, the iMGL of this disclosure may be capable of any of the cell therapeutic effects upon transplantation in a subject described below. The iMGL described herein are capable of responding to pro-inflammatory conditions, including dysregulated accumulation of chemokines and/or cytokines. In some embodiments, the iMGL will respond to disease-associated accumulation of cytokines or chemokines in vivo or in vitro similar to adult microglia. In some embodiments, the iMGL is capable of responding to any stimulus known in the art to prevent dysregulated accumulation of cytokines and/or chemokines in a brain in a manner similar to adult microglia cells. In some embodiments, the accumulated chemokines are any one or more of osteopontin (OPN), TNF□, CCL2 (i.e., MCP-1), CCL4, and CXCL10, in any combination and are secreted in response to stimulation by lipopolysaccharide, IFNγ, or IL-113. In some embodiments, the iMGL is capable of endocytosing accumulated OPN-expression particles in a brain of a subject. It shall be understood that the engineered HPCs and MPCs are capable of differentiating into an iMGL capable of all aforementioned intrinsic and extrinsic cellular functions carried out by natural microglia either in vitro or in vivo.

Isolated Cells for Generating Edited iMGLs, MPCs, HPCs, and MCs

The edited and differentiated cells described herein may be generated by the differentiation and editing of an isolated cell according to the methods described herein. In some instances, the isolated cell is engineered to become an iMGL, MPC, HPC, MC, or macrophage. In some instances, the isolated cell may also be an edited (i.e., genetically edited) isolated cell subjected to the engineered differentiation method described herein. Any of the isolated cells provided herein may harbor the synthetic CSF1R gene described below.

In some embodiments, the edited and differentiated cell is generated from an isolated cell. In some embodiments, the isolated cell is an isolated human cell. In some embodiments, the isolated cell was derived from a stem cell. In some embodiments, the isolated cell was derived from a pluripotent stem cell (PSC). In some embodiments, the isolated cell was derived from an induced pluripotent stem cell (iPSC). In some embodiments, the isolated cell was derived from a hematopoietic stem cell (HSC), a microglial progenitor cell (MPC), a hematopoietic progenitor cell (HPC), or a myeloid cell (MC). In some embodiments, the isolated cell is an HPC. In some embodiments, the isolated cell is an MPC. In some embodiments, the isolated cell is a PSC. In some embodiments, the isolated cell is an iPSC. In some embodiments, the isolated cell is a HSC. In some embodiments, the isolated cell is capable of differentiating into an iMGL. In some embodiments, the isolated cell is capable of differentiating into an MPC. In some embodiments, the isolated cell is capable of differentiating into an HPC. In some embodiments, the isolated cell is capable of differentiating into a myeloid cell. In some embodiments, the isolated cell is capable of differentiating into a macrophage.

### Sources of Isolated Cells

In some embodiments, a sample comprising the isolated cell was collected from a donor. In some embodiments, the sample comprises fibroblasts. In some embodiments, the sample comprises stem cells.

In some embodiments, the sample comprises pluripotent stem cells. In some embodiments, the sample comprises a skin sample. In some embodiments, the sample comprises a bone marrow sample. In some embodiments, the sample comprises a blood sample or a cerebrospinal fluid sample. In some embodiments, the isolated cell was collected from a donor, wherein the donor will receive the isolated cell after the editing and differentiating of the isolated cell in the collected sample. In some embodiments, the isolated cell may be any of the engineered cells of this disclosure.

### Synthetic Csf1R Gene

It shall be understood that any of the synthetic CSF1R genes may be harbored by or edited into any of the engineered cells of the disclosure (e.g., iPSC, PSC, iMGL, MPC, HPC, MC, etc.). In some aspects, the engineered cell described herein may harbor at least one synthetic CSF1R gene used to correct, replace, or repair a defective CSF1R gene in the cells prior to the genetic engineering. In some embodiments, the synthetic CSF1R gene comprises an inserted CSF1R gene or fragment thereof located 3′ of the fms-intronic response element (FIRE). In some embodiments, the synthetic repaired or replaced CSF1R gene comprises a silent mutation. It is important to introduce the synthetic gene 3′ of the FIRE because the FIRE is necessary for transcription of the CSF1R gene within microglia, iMGLs, and the other edited cell types provided in the disclosure. In some embodiments, the synthetic CSF1R gene comprises an open reading frame or portion thereof of a human CSF1R gene. In some cases, the silent mutation is a DNA nucleotide substitution that is inserted into the synthetic gene to prevent recutting by the Cas endonuclease. For example, the synthetic CSF1R gene may have a silent mutation of an adenine as a substitution for guanidine located at the fifth nucleotide position of the nucleotide sequence of SEQ ID NO: 2. In another instance, the synthetic CSF1R gene may have a silent mutation of a thymine or cystine as a substitution for guanidine located at the fifth nucleotide position of the nucleotide sequence of SEQ ID NO: 2. In some embodiments, the synthetic CSF1R gene further comprises a stop codon and a poly-A signal.

cDNA Fragment Characteristics

In some cases, the synthetic CSF1R gene described herein may be, for example, a cDNA fragment inserted into a CSF1R gene locus in the genome of an isolated cell, preferably an isolated cell harboring a mutant CSF1R allele. Precise insertion of the cDNA fragment is facilitated by the DNA templates provided by the nucleic acid sequences of left homology arm (SEQ ID NO: 3) and right homology arm (SEQ ID NO: 4). In some embodiments, the synthetic gene comprises a cDNA fragment of a CSF1R gene, preferably a human CSF1R gene. In some embodiments, the cDNA fragment comprises an open reading frame of a CSF1R gene, preferably a human CSF1R gene. In some embodiments, the cDNA fragment comprises an open reading frame of a CSF1R gene, the silent mutation described herein. In some embodiments, the cDNA fragment lacks a CSF1R start codon. In some embodiments, the cDNA fragment lacks the FIRE or any portion thereof. In some embodiments, the cDNA fragment comprises an open reading frame of a CSF1R gene, the silent mutation, the stop codon, and the poly-A signal. In some embodiments, the cDNA fragment comprises exons 2-21 of a human CSF1R gene. In some embodiments, the cDNA fragment comprises exons 3-21 of the human CSF1R gene. In some embodiments, the cDNA fragment comprises a poly-A signal. In some embodiments, the CSF1R cDNA fragment comprises a nucleic acid sequence having at least 80%, 90%, 95%, 97%, or 99% to the nucleic acid sequence encoding the nucleic acid sequence of SEQ ID NO: 6. In some embodiments, the CSF1R cDNA fragment comprises the nucleic acid sequence of SEQ ID NO: 6. In some embodiments, the CSF1R cDNA fragment consists of the nucleic acid sequence of SEQ ID NO: 6.

### Sequence Similarity to Wildtype CSF1R Protein

In some embodiments, the synthetic CSF1R gene encodes a polypeptide having at least 90% sequence identity to the amino acid sequence of SEQ ID NO: 1 or a fragment thereof. In various aspects, the synthetic CSF1R gene encodes a polypeptide having at least 85% sequence identity to the amino acid sequence of SEQ ID NO: 1 or a fragment thereof. In some embodiments, the synthetic CSF1R gene encodes a polypeptide having at least 97% sequence identity to the amino acid sequence of SEQ ID NO: 1 or a fragment thereof. In some embodiments, the synthetic CSF1R gene encodes a polypeptide having at least 70%, 75%, 80%, 85%, 87%, 90%, 92%, 95%, 96%, 97%, 98%, or 99% sequence identity to the amino acid sequence of SEQ ID NO: 1 or a fragment thereof. The synthetic CSF1R polypeptide can comprise conservative substitutions which do not affect CSF1R function, including but not limited to binding to ligand binding to CSF-1 or IL-34, tyrosine kinase activity, autophosphorylation, autoinhibition, or interactions with src proteins and other downstream signaling molecules.

In various aspects, the synthetic CSF1R gene encodes a polypeptide having at least 95% sequence identity to the amino acid sequence of SEQ ID NO: 1. In various aspects, the synthetic CSF1R gene encodes a polypeptide having at least 90% sequence identity to the amino acid sequence of SEQ ID NO: 1. In various aspects, the synthetic CSF1R gene encodes a polypeptide having at least 85% sequence identity to the amino acid sequence of SEQ ID NO: 1. In various aspects, the synthetic CSF1R gene encodes a polypeptide having at least 80% sequence identity to the amino acid sequence of SEQ ID NO: 1. Nonlimiting examples of the aforementioned polypeptide having at least 95%, 90%, 85%, or 80% sequence identity to the amino acid sequence of SEQ ID NO: 1 are shown in TABLE 8.

In some embodiments, the synthetic CSF1R gene comprises the nucleic acid sequence of SEQ ID NO: 2. In some embodiments, the synthetic CSF1R gene comprises a nucleic acid sequence having at least 70%, 75%, 80%, 85%, 87%, 90%, 92%, 95%, 96%, 97%, 98%, or 99% sequence identity to the nucleotide sequence of SEQ ID NO: 2. In some embodiments, the synthetic CSF1R gene comprises a nucleic acid sequence having at least 90% sequence identity to the nucleotide sequence of SEQ ID NO: 2. In some embodiments, the synthetic CSF1R gene comprises a nucleic acid sequence having at least 97% sequence identity to the nucleotide sequence of SEQ ID NO: 2. In some embodiments, the synthetic CSF1R gene consists of the nucleic acid sequence of SEQ ID NO: 2.

### Function of Synthetic CSF1R Gene

It shall be understood that the synthetic CSF1R gene described herein shall result in improved CSF1R gene expression and function in any of the engineered cell types, which is also increased in comparison to the unmodified isolated source cell (e.g., iPSC, PSC, MPC, HPC, HSC) harboring at least one allele of the defective CSF1R gene described above. In some embodiments, the CSF1R gene expression and function may be almost normal or essentially normal.

In some embodiments, a transcriptional profile of microglia-related genes of the edited and differentiated iMGL harboring the synthetic CSF1R gene more closely resembles a transcriptional profile of the microglia-related genes in a positive control iMGL or microglial progenitor cell (MPC) with exactly two native, wildtype CSF1R alleles compared to a transcriptomic profile of the microglia-related genes in an otherwise identical negative control iMGL or MPC comprising the defective CSF1R gene.

In some embodiments, CSF1R expression in the engineered cell is at least 80% of a CSF1R expression level in an otherwise identical control cell having two native, wildtype CSF1R alleles. In some embodiments, CSF1R expression in the engineered cell is at least 60%, 70%, 75%, 80%, 85%, 90%, 95%, or 100% of a CSF1R expression level in an otherwise identical control cell having two native, wildtype CSF1R alleles.

In some embodiments, CSF1R tyrosine receptor kinase activity in the engineered cell is at least 80% of CSF1R tyrosine receptor kinase activity in an otherwise identical control cell having two native, wildtype CSF1R alleles. CSF1R tyrosine receptor kinase activity in the engineered cell is at least 60%, 70%, 75%, 80%, 85%, 90%, 95%, or 100% of CSF1R tyrosine receptor kinase activity in an otherwise identical control cell having two native, wildtype CSF1R alleles.

In some embodiments, phagocytosis of pro-inflammatory stimuli and byproducts of ALSP pathology in the engineered cell is at least 80% of phagocytosis of pro-inflammatory stimuli and byproducts of ALSP pathology in an otherwise identical control cell having two native, wildtype CSF1R alleles. In some embodiments, phagocytosing pro-inflammatory stimuli and byproducts of ALSP pathology in the engineered cell is at least 60%, 70%, 75%, 80%, 85%, 90%, 95%, or 100% of phagocytosing pro-inflammatory stimuli and byproducts of ALSP pathology in an otherwise identical control cell having two native, wildtype CSF1R alleles. In some embodiments, the byproduct of ALSP pathology comprises hydroxyapatite calcium crystals, SMI312+ axonal spheroids, accumulated or aggregated osteopontin (OPN), Tau+ neurofilaments, or phosphorylated Tau proteins (e.g., Tau phosphorylated at Thr217).

In some embodiments, the frequency of ADP-induced calcium transients in the engineered cell is at least 80% of the frequency of ADP-induced calcium transients in an otherwise identical control cell having two native, wildtype CSF1R alleles. In some embodiments, the frequency of ADP-induced calcium transients in the engineered cell is at least 60%, 70%, 75%, 80%, 85%, 90%, 95%, or 100% of the frequency of ADP-induced calcium transients in an otherwise identical control cell having two native, wildtype CSF1R alleles.

In some embodiments, the proliferation rate in the engineered cell is at least 80% of the proliferation rate in an otherwise identical control cell having two native, wildtype CSF1R alleles. In some embodiments, the proliferation rate in the engineered cell is at least 60%, 70%, 75%, 80%, 85%, 90%, 95%, or 100% of the proliferation rate in an otherwise identical control cell having two native, wildtype CSF1R alleles.

In some embodiments, the frequency of CD9-expressing engineered cells is at least 80% of the frequency of CD9-expressing otherwise-identical control cell having two native, wildtype CSF1R alleles.

In some embodiments, the frequency of CD9-expressing engineered cells is at least 60%, 70%, 75%, 80%, 85%, 90%, 95%, or 100% of the frequency of CD9-expressing otherwise-identical control cell having two native, wildtype CSF1R alleles. CD9 is a known cell marker of activated iMGLs or natural microglia.

In some embodiments, CSF1R expression in the engineered cell is at least 1.2-fold higher than the CSF1R expression level in an unmodified isolated cell with a defective CSF1R gene. In some embodiments, CSF1R expression in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold of a CSF1R expression level in a similar unmodified cell with a defective CSF1R gene.

In some embodiments, CSF1R tyrosine receptor kinase activity in the engineered cell is at least 1.2-fold higher than the CSF1R tyrosine receptor kinase activity in a similar unmodified cell with a defective CSF1R gene. CSF1R tyrosine receptor kinase activity in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the CSF1R tyrosine receptor kinase activity in a similar unmodified cell with a defective CSF1R gene.

In some embodiments, phagocytosis of pro-inflammatory stimuli and byproducts of ALSP pathology by the engineered cell is at least 1.2-fold higher than the phagocytosis of pro-inflammatory stimuli and byproducts of ALSP pathology in a similar unmodified cell with a defective CSF1R gene. In some embodiments, phagocytosis of pro-inflammatory stimuli and byproducts of ALSP pathology by the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold than the phagocytosis of pro-inflammatory stimuli and byproducts of ALSP pathology by a similar unmodified cell with a defective CSF1R gene. In some embodiments, the byproduct of ALSP pathology comprises hydroxyapatite calcium crystals, SM1312+ axonal spheroids, accumulated or aggregated osteopontin (OPN), Tau+ neurofilaments, or phosphorylated Tau proteins (e.g., Tau phosphorylated at Thr217).

In some embodiments, the frequency of ADP-induced calcium transients in the engineered cell is at least 1.2-fold higher than the frequency of ADP-induced calcium transients in a similar unmodified cell with a defective CSF1R gene. In some embodiments, the frequency of ADP-induced calcium transients in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the frequency of ADP-induced calcium transients in a similar unmodified cell with a defective CSF1R gene.

In some embodiments, the proliferation rate of the engineered cell is at least 1.2-fold higher than the proliferation rate of a similar unmodified cell with a defective CSF1R gene. In some embodiments, the proliferation rate in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the proliferation rate in a similar unmodified cell with a defective CSF1R gene.

In some embodiments, the frequency of CD9-expressing engineered cells is at least 1.2-fold higher than the frequency of similar CD9-expressing unmodified cells with a defective CSF1R gene. In some embodiments, the frequency of CD9-expressing engineered cells is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the frequency of similar CD9-expressing unmodified cells with a defective CSF1R gene.

### Defective CSF1R Gene

The disclosure provides methods of replacing or repairing a defective gene (i.e., mutant gene) in the engineered cells described above. In some instances, the mutant gene is a defective CSF1R gene. In some instances, the defective CSF1R gene is preferably a human CSF1R gene. As described below, the defective CSF1R gene harbors mutations that result in defective CSF1R expression and signaling, microglial dysfunction, and aberrant brain function in a subject.

In some embodiments, the mutation in the CSF1R gene encodes a protein with at least one amino acid differing from a wildtype CSF1R protein having the polypeptide sequence of SEQ ID NO: 1 or a fragment thereof. In some embodiments, a protein encoded by the defective CSF1R gene comprises at least one point mutation in the amino acid sequence compared to a naturally occurring wildtype CSF1R protein with the amino acid sequence of SEQ ID NO: 1 or a fragment thereof. In some embodiments, the at least one mutation comprises a point mutation. In some embodiments, the point mutation in the defective CSF1R gene comprises a single nucleotide polymorphism (SNP). In some embodiments, the mutation comprises a point mutation encoding the amino acid residue methionine-875 (M875). In some embodiments, the point mutation encoding M875 comprises an amino acid substitution from methionine to isoleucine (M875I). In some embodiments, the point mutation encoding M875 comprises an amino acid substitution from methionine to threonine (M875T). In some embodiments, the mutation comprises a point mutation encoding the amino acid residue leucine-786 (L786). In some embodiments, the point mutation encoding the L786 mutation comprises an amino acid substitution from leucine to serine (L786S). In some embodiments, the mutation comprises a point mutation encoding the amino acid residue asparagine-854 (N854). In some embodiments, the point mutation encoding the N854 mutation comprises an amino acid substitution from asparagine to lysine (N854K). In some embodiments, the mutation comprises a point mutation encoding the amino acid residue glycine-589 (G589). In some embodiments, the point mutation encoding the G589 mutation comprises an amino acid substitution from glycine to glutamate (G589E). In some embodiments, the mutation comprises a point mutation encoding the amino acid residue glycine-585 (G585). In some embodiments, the point mutation encoding the G589 mutation comprises an amino acid substitution from glycine to valine (G585V). In some embodiments, the mutation comprises a point mutation encoding the amino acid residue glutamine-481 (G481). In some embodiments, the mutation comprises a point mutation encoding the amino acid residue proline-132 (P132). In some embodiments, the point mutation encoding the P132 mutation comprises an amino acid substitution from proline to leucine (P132L). In some embodiments, the mutation comprises a point mutation encoding the amino acid residue tyrosine-540 (Y540). In some embodiments, the mutation comprises a point mutation encoding the amino acid residue lysine-627 (K627). In some embodiments, the mutation comprises a point mutation encoding the amino acid residue histidine-643 (H643). In some embodiments, the point mutation encoding the H643 mutation comprises an amino acid substitution from histidine to glutamine (H643Q). In some embodiments, the mutation comprises a point mutation encoding the amino acid residue threonine-833 (T833). In some embodiments, the point mutation encoding the T833 mutation comprises an amino acid substitution from threonine to methionine (T833M). In some embodiments, the mutation can comprise any one of the CSF1R mutations described in Dulski et al., Brain abnormalities, neurodegeneration, and dysosteosclerosis (BANDDOS): new cases, systematic literature review, and associations with CSF1R-ALSP, Orphanet Journal of Rare Diseases (2023).

In some embodiments, the mutation in the defective CSF1R gene causes a disease-associated phenotype. In some embodiments, the disease-associated phenotype comprises increased accumulation of secreted osteopontin (OPN) in the brain of a subject compared to a subject lacking the defective CSF1R gene. In some embodiments, the disease-associated phenotype comprises increased frequency of axonal spheroids immunoreactive for neurofilament SMI312 and LAMP1 in the brain of a subject compared to a subject lacking the defective CSF1R gene. In some embodiments, the disease-associated phenotype comprises increased number, density, or frequency of hydroxyapatite calcium crystals in the brain of a subject compared to a subject lacking the defective CSF1R gene. In some embodiments, the disease-associated phenotype comprises increased calcification of the brain of a subject compared to a subject lacking the defective CSF1R gene. In some embodiments, the disease-associated phenotype comprises increased expression levels of Tau phosphorylated at Thr217 (pTau217) in the brain of a subject compared to a subject lacking the defective CSF1R gene. In some embodiments, the disease-associated phenotype comprises increased expression levels of Tau phosphorylated at Thr217 (pTau217) in the blood plasma or cerebrospinal fluid of a subject compared to a subject lacking the defective CSF1R gene. In some embodiments, the disease-associated phenotype comprises increased GFAP expression levels in the brain of a subject compared to a subject lacking the defective CSF1R gene. In some embodiments, the disease-associated phenotype comprises increased GFAP expression levels in the blood plasma or cerebrospinal fluid of a subject compared to a subject lacking the defective CSF1R gene. In some embodiments, the disease-associated phenotype comprises increased MCP-1 expression levels in the brain of a subject compared to a subject lacking the defective CSF1R gene. In another instance, the disease-associated phenotype may cause increased expression levels of at least one of SERPINA3N, neurofilament light chain (NFL), GFAP, pTau217, MCP-1 (CCL2), Osteopontin (SPP1), LAMP1, LAMP2, or any combination thereof in the brain, blood, or cerebrospinal of a subject compared to a subject lacking the defective CSF1R gene. In another instance, the disease-associated phenotype may cause increased expression levels of at least two of SERPINA3N, neurofilament light chain (NFL), GFAP, pTau217, MCP-1 (CCL2), Osteopontin (SPP1), LAMP1, or LAMP2 in the brain, blood, or cerebrospinal of a subject compared to a subject lacking the defective CSF1R gene. In some embodiments, the disease-associated phenotype comprises decreased expression of synaptic proteins in the brain of a subject compared to a subject lacking the defective CSF1R gene. In some embodiments, the disease-associated phenotype comprises decreased expression of neuronal proteins in the brain of a subject compared to a subject lacking the defective CSF1R gene. In some embodiments, the disease-associated phenotype comprises a decreased number or density of synapses in the brain of a subject compared to a subject lacking the defective CSF1R gene. In some embodiments, the disease-associated phenotype comprises a decreased average size of synapses in the brain of a subject compared to a subject lacking the defective CSF1R gene.

Generating Engineered Cells In Vitro iMGLs, MPCs, and HPCs

A cell therapy consisting of the engineered cells (e.g., MLG, MPC, HPC, MC) described above may be generated from an isolated cell capable of undergoing further differentiation, whereby the isolated cell is subjected to editing (i.e., genetic editing) to repair or replace a target gene (e.g., defective CSF1R gene, etc.) in the isolated cell and then incubating the isolated cell in a culture media comprising a differentiation factor in order to generate an edited and differentiated cell which is derived from the isolated cell. The editing may include, but is not limited to, editing the genome of the isolated cell such that the target cell is corrected, repaired, or replaced resulting in the isolated cell harboring any of the synthetic genes described above (e.g., synthetic CSF1R gene). The incubating of the isolated cell is performed in vitro such that the isolated cell can be further differentiated into the engineered cell (e.g., iMGL, MPC, or HPC).

Provided herein is a method of preparing a cell therapy, the method comprising: editing the genome of an isolated cell to repair or replace a target gene; and incubating the isolated cell in a culture media comprising a differentiation factor, thereby generating an edited and differentiated cell.

It shall be understood that the target gene to be corrected, repaired, or replaced by the method of preparing a cell therapy described herein may be any of the defective genes or any gene harboring any of the disease-associated mutations described above (e.g., defective human CSF1R gene, ALSP-linked gene mutation encoding human CSF1R-L786S protein, etc.).

In some embodiments, the target gene comprises a disease-associated mutation. In some embodiments, the target gene comprises a defective human CSF1R gene. In some embodiments, the target gene comprises a mutation located 3′ of a fms-intronic response element (FIRE) in the CSF1R gene. In some embodiments, the disease-associated mutation comprises a mutation of a CSF1R gene.

In some embodiments, the isolated cell is an isolated human cell. In some embodiments, the isolated cell was derived from a stem cell. In some embodiments, the isolated cell was derived from an iPSC. In some embodiments, the isolated cell was derived from a hematopoietic stem cell (HSC), a hematopoietic precursor cell (HPC), or a myeloid cell.

The edited and differentiated cell generated by the method described herein can be any of the engineered cells of this disclosure (e.g., iMGL, MPC, HPC, MC, macrophage, etc.), whereby the undifferentiated cell may include, for example, a PSC, iPSC, HSC, etc. The isolated cell to be edited and differentiated according to the method described herein can be any of the isolated cells described above. In some embodiments, the edited and differentiated cell is a myeloid cell, a myeloid precursor cell, a myeloid progenitor cell, an erythro-myeloid precursor cell, an erythro-myeloid progenitor cell, a macrophage, a monocyte, a fetal macrophage, a hematopoietic stem cell-derived myeloid cell, or a yolk-sac-derived myeloid cell. In some embodiments, the edited and differentiated cell is an HPC or a microglial precursor cell. In some embodiments, the edited and differentiated cell is an iMGL.

In some embodiments, the method further comprises generating iPSCs before the editing or the incubating. In some embodiments, the editing comprises repairing a genetic mutation. In some embodiments, the editing comprises inserting an open reading frame within the target gene in the isolated cell. In some embodiments, the method further comprises culturing and expanding the edited and differentiated cell. The culturing and expanding the edited and differentiated cell will generate a cell population comprising mostly of the edited and differentiated cell.

### Gene Editing

In various aspects, any mutant microglial genes (e.g., defective CSF1R gene) present in the cells of the sample of cells may be repaired, corrected, or replaced in the subject-derived iPSCs using gene editing techniques known in the art. Suitable gene editing techniques may include any gene editing system that is capable of repairing, correcting, or replacing mutant versions of the microglial gene. In some aspects, suitable gene editing techniques may include conventional genome editing systems, such as conventional homologous recombination, ssODNs homologous recombination; chemical systems, such as peptide NA systems; protein based nuclease systems, such as meganuclease systems, zinc-finger nuclease systems and TALEN systems; homing endonuclease (HE) systems, such as AdenoAssociated Virus (AAA) systems; and RNA protein based systems, such as CRISPR systems, and the like. In some embodiments, the editing comprises contacting the mutant CSF1R gene with a TALEN, a zinc-finger endonuclease, a Cas endonuclease or a meganuclease. In some embodiments, the CRISPR system may comprise a base editor or a prime editor.

### Crispr Gene Editing

Preferably, the engineered cells described above are genetically edited by conventional single guide-RNA (gRNA) CRISPR-mediated gene editing methods for inserting gene fragments into a target gene (e.g., defective CSF1R gene) in order to correct or restore the expression levels of the target gene to otherwise natural wildtype expression levels. It shall be understood that any of the engineered cells of this disclosure can harbor the corrected, replacement, or repaired target gene (e.g., defective CSF1R gene) and can be any of the edited and engineered cells generated from the isolated cells (e.g., iPSCs, PSCs, HPCs, iMGLs, MPCs, HSCs, etc.) according to the methods described herein.

In some embodiments, any one of the engineered cell types of this disclosure is genetically edited by CRISPR/Cas gene editing methods. In some embodiments, CRISPR/Cas gene editing comprises a Cas endonuclease. In some embodiments, the Cas endonuclease comprises a Cas12a, Cas13, or Cas9 endonuclease. In some embodiments, CRISPR/Cas gene editing comprises a Cas9 endonuclease, a catalytically dead Cas9 endonuclease, or a nickase Cas9 endonuclease. In some embodiments, CRISPR/Cas gene editing comprises a M-MLV reverse transcriptase (RT) fused to a nickase Cas9 endonuclease. In some embodiments, the editing or engineering comprises a CRISPR/Cas9-mediated correction of a defective CSF1R gene.

### CRISPR Base Editing Systems

In some embodiments, the CRISPR system may comprise a base editor or base editing. A base editor is a newly developed tool able to precisely edit DNA sequences in a specific locus without inducing double-stranded breaks in DNA to generate adenine-to-guanine, cytosine-to-thymine, and cytosine-to-guanidine substitutions; this is especially efficient for correcting point mutations in a gene (e.g., SNPs), for example. Different base editors have been created allowing base conversions in a variety of target regions. For example, the cytosine base editors allow the conversion of a cytosine:guanine base pair to a thymine:adenine base pair. Additionally, adenine base editors convert an adenine:thymine base pair into a guanine:cytosine base pair. Base editors are composed by a catalytically dead Cas9 (dCas9) or a nickase Cas9 (nCas9) fused to a deaminase and guided by a single guide RNA (sgRNA) to the locus of interest (e.g., point mutations in a defective CSF1R gene). The dCas9 or nCas9 recognizes a specific sequence called a protospacer adjacent motif (PAM), and the DNA unwinds due to the complementarity between the sgRNA, and the DNA sequence usually located upstream of the PAM (“protospacer”). Then, the opposite DNA strand is accessible to the deaminase that converts the bases located in a specific DNA stretch of the protospacer. (See Antoniou et al., Base and Prime Editing Technologies for Blood Disorders, Frontiers in Genome Editing (2021) Volume 3-2021.)

### CRISPR Prime Editing Systems

In some embodiments, the CRISPR system may comprise a prime editor or prime editing. The prime editing CRISPR system retains the targeting specificity of CRISPR and carries additional cargo in the form of an edit-containing RNA template as a contiguous extension of the guide RNA (known as a prime editing guide RNA, “pegRNA”), and a M-MLV reverse transcriptase (RT) fused to the C terminus of Cas9 (H840A) nickase. Use of the Cas9 nickase avoids the formation of a double-stranded DNA break, and simply cuts the non-complementary strand of the DNA three bases upstream of the PAM site. This exposes a DNA flap with a 3′ OH group which binds to the primer binding site of the RNA template, serving as a primer for RT, which extends the 3′ flap by copying the edit sequence of the pegRNA. Despite this extended 3′ flap being thermodynamically less likely to hybridize to the unedited complementary strand compared to the unedited 5′ flap, the inherent preference of the endogenous endonuclease FEN1 to excise 5′ flaps leads to hybridization of the edited 3′ flap being favored, thus resulting in highly efficient base editing useful for correcting point mutations in a gene of interest, including but not limited to a defective CSF1R gene. Scholefield, J., Harrison, P.T. Prime editing—an update on the field. Gene Therapy 28, 396-401 (2021)

### Summary of CRISPR Strategies

As described below, the synthetic gene described herein may be introduced to the genome of an isolated source cell (e.g., iPSC, PSC, myeloid cell) or engineered cell (e.g., iMGL, HPC, MPC, macrophage, etc.) of the disclosure at a target gene locus (e.g., defective CSF1R gene). CRISPR/Cas9-mediated correction of a defective CSF1R gene may include inserting a cDNA fragment encoding a corrected protein capable of expression levels and protein function essentially equivalent to the expression levels and function of the wildtype version of the protein. Alternatively, the CRISPR/Cas9-mediated correction of a defective CSF1R gene may include repairing any of the disease-associated point mutations described above via homology-directed repair mechanisms utilizing a single guide RNA (sgRNA) and a homology-directed repair template polynucleotide in the presence of any Cas endonuclease described herein. In some cases, the homology-directed repair template polynucleotide may be a single-stranded oligodeoxynucleotide (ssODN). In some cases, the CRISPR/Cas9-mediated correction of a defective CSF1R gene may include repairing any of the disease-associated point mutations described above via the base editing or prime editing CRISPR systems described above.

CSF1R insertion Strategy

In one aspect, the synthetic gene described herein is introduced into any of the isolated cell types described above (e.g., iPSCs, PSCs, HPCs, MPC, iMGLs, HSCs, myeloid cells) by editing a target gene (e.g., defective CSF1R gene) to insert the cDNA fragment described herein encoding a protein, for example but not limited to, a human CSF1R protein, capable of normal wildtype CSF1R protein expression level and CSF1R protein function (e.g., CSF1R tyrosine receptor kinase activity). The editing of the target gene (e.g., defective CSF1R gene), as described below, may require inserting the cDNA fragment described herein into the CSF1R gene locus of an isolated cell (e.g., iPSC, PSC, HPC, HSC, iMGL, MPC, myeloid cells). In such cases, the cDNA fragment can be inserted between the left homology arm and a right homology arm flanking a portion of the defective CSF1R gene locus located 3′ of the fms-intronic response element (FIRE) of the CSF1R. In some cases, the left homology arm may have a nucleic acid sequence having a sequence identity of at least 80%, 90%, 95%. 97%, or 99% to the nucleic acid sequence of SEQ ID NO: 3. In some cases, the left homology arm may consist of the nucleic acid sequence of SEQ ID NO: 3. In some cases, the right homology arm may have a nucleic acid sequence having a sequence identity of at least 80%, 90%, 95%, 97%, or 99% to the nucleic acid sequence of SEQ ID NO: 4. In some cases, the right homology arm may consist of the nucleic acid sequence of SEQ ID NO: 4. In some embodiments, the synthetic CSF1R gene comprises the nucleic acid sequence of SEQ ID NO: 2. In some embodiments, the synthetic CSF1R gene comprises a nucleic acid sequence having at least 70%, 75%, 80%, 85%, 87%, 90%, 92%, 95%, 96%, 97%, 98%, or 99% sequence identity to the nucleotide sequence of SEQ ID NO: 2.

In some embodiments, the synthetic CSF1R gene comprises a nucleic acid sequence having at least 90% sequence identity to the nucleotide sequence of SEQ ID NO: 2. In some embodiments, the synthetic CSF1R gene comprises a nucleic acid sequence having at least 97% sequence identity to the nucleotide sequence of SEQ ID NO: 2. In some embodiments, the synthetic CSF1R gene consists of the nucleic acid sequence of SEQ ID NO: 2.

In some embodiments, the editing comprises introducing into the cell: a Cas9 polypeptide or a polynucleotide encoding the Cas9 polypeptide, a gRNA comprising a sequence of the CSF1R gene or a polynucleotide encoding the gRNA, and a polynucleotide comprising a CSF1R cDNA fragment, thereby replacing the CSF1R gene. In some embodiments, the gRNA comprises the nucleic acid sequence of SEQ ID NO: 5. In some embodiments, the gRNA comprises a nucleic acid sequence comprising at least 90% sequence identity to the nucleic acid sequence of SEQ ID NO: 5.

### CSF1R Repairing Strategy

In another aspect, the target gene (e.g., defective CSF1R gene) may be repaired to eliminate a point mutation, preferably a disease-associated CSF1R point mutation, including but not limited to any of the disease-associated point mutations described above (e.g., M875, L786, M875, N854, G589, M8751, L786S, M875T, N854K, G589E, G481, P132L, Y540, K627, H643, H643Q, T833, T833M, etc.), in the genome of the isolated cell. A preferred example of repairing the target gene includes, but is not limited to, the CRISPR/Cas9-mediated correction of a defective CSF1R gene may include repairing any of the disease-associated point mutations described above via homology-directed repair mechanisms by utilizing a single guide RNA (gRNA) and a homology-directed repair template polynucleotide in the presence of any Cas endonuclease described herein. In some cases, the homology-directed repair template polynucleotide may be a single-stranded oligodeoxynucleotide (ssODN). In some embodiments, the repairing the target gene includes a CRISPR base editor or a CRISPR prime editor as described above.

In some embodiments, the editing comprises any of the gene editing methods provided herein to repair a target gene, preferably a defective CSF1R gene. In some embodiments, the editing comprises contacting the target gene with a TALEN, a zinc-finger endonuclease, or a meganuclease. In some embodiments, the editing comprises contacting the target gene with a CRISPR endonuclease. In some embodiments, the CRISPR endonuclease comprises Cas9. In some embodiments, the Cas endonuclease comprises a Cas12a, Cas13, or Cas9 endonuclease. In some embodiments, the CRISPR/Cas gene editing comprises a Cas9 endonuclease, a catalytically dead Cas9 endonuclease, or a nickase Cas9 endonuclease.

In some embodiments, CRISPR/Cas gene editing comprises a M-MLV reverse transcriptase (RT) fused to a nickase Cas9 endonuclease.

In some embodiments, the repairing further comprises introducing into the cell: a Cas9 polypeptide or a polynucleotide encoding the Cas9 polypeptide, a gRNA complementary to a nucleotide sequence adjacent to the nucleotide sequence encoding the point mutation, and a homology-directed repair template polynucleotide comprising a wildtype CSF1R sequence at a position in the CSF1R gene corresponding to the position of the point mutation, thereby repairing the point mutation. In some embodiments, the repairing further comprises introducing a pegRNA into the cell. In some embodiments, the repairing further comprises introducing into the cell: a catalytically dead Cas9 endonuclease, or a nickase Cas9 endonuclease. In some embodiments, CRISPR/Cas gene editing comprises a M-MLV reverse transcriptase (RT) fused to a nickase Cas9 endonuclease. In some embodiments, the repairing further comprises introducing a pegRNA into the cell. In some embodiments, the homology-directed repair template polynucleotide is a single-stranded DNA oligonucleotide (ssODN).

In some embodiments, the point mutation comprises the M875I mutation of this disclosure. As disclosed herein the M875I point mutation may be corrected, in some embodiments, with the gRNA comprising the nucleic acid sequence of SEQ ID NO: 12, or alternatively, a gRNA comprising a nucleic acid sequence with at least 90% sequence identity to the nucleic acid sequence of SEQ ID NO: 12. In some embodiments, the M8751 point mutation may be corrected when the repairing comprises the homology-directed repair template polynucleotide comprising the nucleic acid sequence of SEQ ID NO: 7. In some embodiments, the M8751 point mutation may be corrected when the repairing comprises the homology-directed repair template polynucleotide comprising at least 90% or 95% sequence identity to the nucleic acid sequence of SEQ ID NO: 7. In some embodiments, the M875I point mutation may be corrected when the repairing comprises introducing into the cell: (i) a gRNA comprising the nucleic acid sequence of SEQ ID NO: 12, and (ii) the homology-directed repair template polynucleotide comprising the nucleic acid sequence of SEQ ID NO: 7.

In some embodiments, the point mutation comprises the L786S mutation of this disclosure. As disclosed herein the L786S point mutation may be corrected, in some embodiments, the gRNA comprising the nucleic acid sequence of SEQ ID NO: 13, or alternatively, a gRNA comprising a nucleic acid sequence with at least 90% sequence identity to the nucleic acid sequence of SEQ ID NO: 13. In some embodiments, the L786S point mutation may be corrected when the repairing comprises the homology-directed repair template polynucleotide comprising the nucleic acid sequence of SEQ ID NO: 8. In some embodiments, the L786S point mutation may be corrected when the repairing comprises the homology-directed repair template polynucleotide comprising at least 90% or 95% sequence identity to the nucleic acid sequence of SEQ ID NO: 8. In some embodiments, the L786S point mutation may be corrected when the repairing comprises introducing into the cell: (i) a gRNA comprising the nucleic acid sequence of SEQ ID NO: 13, and (ii) the homology-directed repair template polynucleotide comprising the nucleic acid sequence of SEQ ID NO: 8.

In some embodiments, the point mutation comprises the M875T mutation of this disclosure. As disclosed herein the M875T point mutation may be corrected, in some embodiments, with the gRNA comprising the nucleic acid sequence of SEQ ID NO: 14, or alternatively, a gRNA comprising a nucleic acid sequence with at least 90% sequence identity to the nucleic acid sequence of SEQ ID NO: 14. In some embodiments, the M875T point mutation may be corrected when the repairing comprises the homology-directed repair template polynucleotide comprising the nucleic acid sequence of SEQ ID NO: 9. In some embodiments, the M875T point mutation may be corrected when the repairing comprises the homology-directed repair template polynucleotide comprising at least 90% or 95% sequence identity to the nucleic acid sequence of SEQ ID NO: 9. In some embodiments, the M875T point mutation may be corrected when the repairing comprises introducing into the cell: (i) a gRNA comprising the nucleic acid sequence of SEQ ID NO: 14, and (ii) the homology-directed repair template polynucleotide comprising the nucleic acid sequence of SEQ ID NO: 9.

In some embodiments, the point mutation comprises the N854K mutation of this disclosure. As disclosed herein the N854K point mutation may be corrected, in some embodiments, the gRNA comprising the nucleic acid sequence of SEQ ID NO: 15, or alternatively, a gRNA comprising a nucleic acid sequence with at least 90% sequence identity to the nucleic acid sequence of SEQ ID NO: 15. In some embodiments, the N854K point mutation may be corrected when the repairing comprises the homology-directed repair template polynucleotide comprising the nucleic acid sequence of SEQ ID NO: 10. In some embodiments, the N854K point mutation may be corrected when the repairing comprises the homology-directed repair template polynucleotide comprising at least 90% or 95% sequence identity to the nucleic acid sequence of SEQ ID NO: 10. In some embodiments, the N854K point mutation may be corrected when the repairing comprises introducing into the cell: (i) a gRNA comprising the nucleic acid sequence of SEQ ID NO: 15, and (ii) the homology-directed repair template polynucleotide comprising the nucleic acid sequence of SEQ ID NO: 10.

In some embodiments, the point mutation comprises the G589E mutation of this disclosure. As disclosed herein the G589E point mutation may be corrected, in some embodiments, with the gRNA comprising the nucleic acid sequence of SEQ ID NO: 16, or alternatively, a gRNA comprising a nucleic acid sequence with at least 90% sequence identity to the nucleic acid sequence of SEQ ID NO: 16. In some embodiments, the G589E point mutation may be corrected when the repairing comprises the homology-directed repair template polynucleotide comprising the nucleic acid sequence of SEQ ID NO: 11. In some embodiments, the G589E point mutation may be corrected when the repairing comprises the homology-directed repair template polynucleotide comprising at least 90% or 95% sequence identity to the nucleic acid sequence of SEQ ID NO: 11. In some embodiments, the G589E point mutation may be corrected when the repairing comprises introducing into the cell: (i) a gRNA comprising the nucleic acid sequence of SEQ ID NO: 16, and (ii) the homology-directed repair template polynucleotide comprising the nucleic acid sequence of SEQ ID NO: 11.

### Function of the Repaired CSF1R Gene or the Inserted Synthetic CSF1R Gene

The expression of the repaired CSF1R gene described herein can result in essentially normal CSF1R gene expression and function in any of the engineered cell types (e.g., iPSC, PSC, HPC, HSC, iMGL, MPC, myeloid cell), which is also increased in comparison to the unmodified isolated cell harboring at least one allele of the defective CSF1R gene described above.

In some embodiments, a transcriptional profile of microglia-related genes of the edited and differentiated iMGL or MPC harboring the synthetic CSF1R gene or repaired CSF1R gene more closely resembles a transcriptional profile of the microglia-related genes in a positive control iMGL or microglial precursor cell with exactly two native, wildtype CSF1R alleles compared to a transcriptomic profile of the microglia-related genes in an otherwise identical negative control iMGL or MPC comprising the defective CSF1R gene.

In some embodiments, CSF1R expression in the engineered cell is at least 80% of a CSF1R expression level in an otherwise identical control cell having two native, wildtype CSF1R alleles. In some embodiments, CSF1R expression in the engineered cell is at least 60%, 70%, 75%, 80%, 85%, 90%, 95%, or 100% of a CSF1R expression level in an otherwise identical control cell having two native, wildtype CSF1R alleles.

In some embodiments, CSF1R tyrosine receptor kinase activity in the engineered cell is at least 80% of CSF1R tyrosine receptor kinase activity in an otherwise identical control cell having two native, wildtype CSF1R alleles. CSF1R tyrosine receptor kinase activity in the engineered cell is at least 60%, 70%, 75%, 80%, 85%, 90%, 95%, or 100% of CSF1R tyrosine receptor kinase activity in an otherwise identical control cell having two native, wildtype CSF1R alleles.

In some embodiments, phagocytosing pro-inflammatory stimuli and byproducts of ALSP pathology in the engineered cell is at least 80% of phagocytosing pro-inflammatory stimuli and byproducts of ALSP pathology in an otherwise identical control cell having two native, wildtype CSF1R alleles. In some embodiments, phagocytosing pro-inflammatory stimuli and byproducts of ALSP pathology in the engineered cell is at least 60%, 70%, 75%, 80%, 85%, 90%, 95%, or 100% of phagocytosing pro-inflammatory stimuli and byproducts of ALSP pathology in an otherwise identical control cell having two native, wildtype CSF1R alleles. In some embodiments, the byproduct of ALSP pathology comprises hydroxyapatite calcium crystals, SMI312+ axonal spheroids, accumulated or aggregated osteopontin (OPN), Tau+ neurofilaments, or phosphorylated Tau proteins (e.g., Tau phosphorylated at Thr217).

In some embodiments, the frequency of ADP-induced calcium transients in the engineered cell is at least 80% of the frequency of ADP-induced calcium transients in an otherwise identical control cell having two native, wildtype CSF1R alleles. In some embodiments, the frequency of ADP-induced calcium transients in the engineered cell is at least 60%, 70%, 75%, 80%, 85%, 90%, 95%, or 100% of the frequency of ADP-induced calcium transients in an otherwise identical control cell having two native, wildtype CSF1R alleles.

In some embodiments, the proliferation rate in the engineered cell is at least 80% of the proliferation rate in an otherwise identical control cell having two native, wildtype CSF1R alleles. In some embodiments, the proliferation rate in the engineered cell is at least 60%, 70%, 75%, 80%, 85%, 90%, 95%, or 100% of the proliferation rate in an otherwise identical control cell having two native, wildtype CSF1R alleles.

In some embodiments, the frequency of CD9-expressing engineered cells is at least 80% of the frequency of CD9-expressing otherwise-identical control cell having two native, wildtype CSF1R alleles.

In some embodiments, the frequency of CD9-expressing engineered cells is at least 60%, 70%, 75%, 80%, 85%, 90%, 95%, or 100% of the frequency of CD9-expressing otherwise-identical control cell having two native, wildtype CSF1R alleles. CD9 is a known cell marker of activated iMGLs or natural microglia.

In some embodiments, CSF1R expression in the engineered cell is at least 1.2-fold higher than the CSF1R expression level in an unmodified isolated cell with a defective CSF1R gene. In some embodiments, CSF1R expression in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold of a CSF1R expression level in a similar unmodified cell with a defective CSF1R gene.

In some embodiments, CSF1R tyrosine receptor kinase activity in the engineered cell is at least 1.2-fold higher than the CSF1R tyrosine receptor kinase activity in a similar unmodified cell with a defective CSF1R gene. CSF1R tyrosine receptor kinase activity in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the CSF1R tyrosine receptor kinase activity in a similar unmodified cell with a defective CSF1R gene.

In some embodiments, phagocytosis of pro-inflammatory stimuli and byproducts of ALSP pathology by the engineered cell is at least 1.2-fold higher than the phagocytosis of pro-inflammatory stimuli and byproducts of ALSP pathology in a similar unmodified cell with a defective CSF1R gene. In some embodiments, phagocytosis of pro-inflammatory stimuli and byproducts of ALSP pathology by the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold than the phagocytosis of pro-inflammatory stimuli and byproducts of ALSP pathology by a similar unmodified cell with a defective CSF1R gene. In some embodiments, the byproduct of ALSP pathology comprises hydroxyapatite calcium crystals, SMJ312+ axonal spheroids, accumulated or aggregated osteopontin (OPN), Tau+ neurofilaments, or phosphorylated Tau proteins (e.g., Tau phosphorylated at Thr217).

In some embodiments, the frequency of ADP-induced calcium transients in the engineered cell is at least 1.2-fold higher than the frequency of ADP-induced calcium transients in a similar unmodified cell with a defective CSF1R gene. In some embodiments, the frequency of ADP-induced calcium transients in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the frequency of ADP-induced calcium transients in a similar unmodified cell with a defective CSF1R gene.

In some embodiments, the proliferation rate of the engineered cell is at least 1.2-fold higher than the proliferation rate of a similar unmodified cell with a defective CSF1R gene. In some embodiments, the proliferation rate in the engineered cell is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold. 2.4-fold, or 2.5-fold higher than the proliferation rate in a similar unmodified cell with a defective CSF1R gene.

In some embodiments, the frequency of CD9-expressing engineered cells is at least 1.2-fold higher than the frequency of similar CD9-expressing unmodified cells with a defective CSF1R gene. In some embodiments, the frequency of CD9-expressing engineered cells is at least 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher than the frequency of similar CD9-expressing unmodified cells with a defective CSF1R gene.

### Engineered Differentiation of Isolated Cells

The disclosed method of preparing a cell therapy comprises: editing the genome of an isolated cell to repair or replace a target gene; and incubating the isolated cell in a culture media comprising a differentiation factor, thereby generating an edited and differentiated cell.

The method requires the differentiation of any isolated cell present herein, preferably HPCs, into MPCs or iMGLs in vitro, and necessarily includes incubating the isolated cell in a culture media comprising a differentiation factor, thereby generating an edited and differentiated cell, preferably the iMGL or MPC of this disclosure. In some embodiments, the editing occurs before the incubating. In some embodiments, the editing occurs after the incubating. In some embodiments, the edited and differentiated cell can differentiate further in vivo.

In some embodiments, the incubating comprises a first incubation period for differentiating an iPSC or PSC into an HPC and a second incubation period for differentiating the HPC.

It shall be understood that the differentiating of isolated cells, including but not limited to, iPSCs and PSC, into HPCs is an optional first step of the method. HPCs used in the method of preparing a cell therapy as described herein may be generated by any method known in the art. HPCs may be generated from any source, including but not limited to a commercially available kit or cell line of PSCs or iPSCs or by the methods of differentiating PSCs or iPSCs into HPCs as described in U.S. patent application Ser. No. 16/489,338, U.S. patent application Ser. No. 14/986,224, Bennett et al. New tools for studying microglia in the mouse and human CNS,” PNAS (2016), Abud et al., iPSC-Derived Human Microglia-like Cells to Study Neurological Diseases Neuron (2017), or McQuade et al., Development and validation of a simplified method to generate human microglia from pluripotent stem cells Molecular Neurodegeneration (2018).

In some embodiments, the isolated cell was derived from a sample collected from a donor. In various aspects, a sample of cells can be collected from a donor using techniques known in the art, such as biopsy, or the like. In some aspects, the sample of cells can include somatic cells, including blood or skin fibroblasts, which can be collected for iPSC generation. In various aspects, the sample of cells can be reprogrammed into iPSCs using techniques known in the art. Suitable reprogramming systems include integrative viral vector transfer systems, integrative nonviral transfer systems, non-integrative viral transfer systems, nonintegrative nonviral transfer systems, and CRISPR systems. In some embodiments, the sample comprises fibroblasts. In some embodiments, the sample comprises stem cells. In some embodiments, the sample comprises pluripotent stem cells. In some embodiments, the sample comprises a skin sample. In some embodiments, the sample comprises a bone marrow sample. In some embodiments, the sample comprises a blood sample or a cerebrospinal fluid sample. In some embodiments, the isolated cell was collected from a donor, wherein the donor will receive the isolated cell after the editing and differentiating of the isolated cell in the collected sample.

Optional Step: Differentiating PSCs or iPSCs into HPCs

The method can optionally include an initial step to generate HPCs according to a method comprising differentiating a pluripotent stem cell (PSC) or a human induced pluripotent stem cell (iPSC) using a media supplemented with hematopoietic differentiation factors to produce induced hematopoietic progenitor cells (HPCs). In some embodiments, the differentiating comprises differentiating PSCs or iPSCs using a media supplemented with hematopoietic differentiation factors. In some embodiments, the PSCs iPSCs may be derived from a tissue sample of a donor or may an acquired from a commercial source of PSCs or iPSCs.

In some embodiments, a sample comprising the isolated cell was collected from a donor. In some embodiments, the sample comprises fibroblasts. In some embodiments, the sample comprises stem cells.

In some embodiments, the sample comprises pluripotent stem cells. In some embodiments, the sample comprises a skin sample. In some embodiments, the sample comprises a bone marrow sample. In some embodiments, the sample comprises a blood sample or a cerebrospinal fluid sample.

In some aspects, the method of generating iMGLs comprises a first incubation period for differentiating a PSC into a microglial progenitor cell. In some embodiments, the method of generating iMGLs comprises: an incubation period for differentiating the HPC or into an iMGL or MPC. In some embodiments, the method of generating iMGLs or MPCs comprises: (i) a first incubation period for differentiating a PSC into an HPC; and (ii) a second incubation period for differentiating the HPC into an iMGL. It shall also be understood that an HPC is a cell type generated by the first step of the differentiation method provided herein. In some aspects, the method of generating iMGLs comprises a first incubation period for differentiating an iPSC into an HPC. In some embodiments, the method of generating iMGLs comprises: (i) a first incubation period for differentiating an iPSC into an HPC; and (ii) a second incubation period for differentiating the HPC into an iMGL or MPC. In some aspects, the method of generating iMGLs or MPCs comprises a first incubation period for differentiating an iPSC into a hematopoietic progenitor cell (HPC). In some embodiments, the method of generating iMGLs or MPCs comprises: (i) a first incubation period for differentiating an iPSC into an HPC; and (ii) a second incubation period for differentiating the HPC.

Steps of In Vitro Differentiation Method: IPSC-derived HPC to iMGL or MPC

In some aspects, the iMGL or MPCs of the disclosure may be generated by the steps of: (i) differentiating a pluripotent stem cell or a human induced pluripotent stem cell using a media supplemented with hematopoietic differentiation factors to produce induced hematopoietic progenitor cells (iHPCs); (ii) optionally isolating CD43+ iHPCs; (iii) differentiating the CD43+ iHPCs into human iMGLs or MPCs using a microglial differentiating media; and (iv) optionally maturing the iMGLs and MPCs in vitro. In some aspects, HPC generation technology allows for collecting media enriched with precursors and carried to (iii) without isolating CD43+ iHPCs.

In some aspects, the human microglial-like cells (iMGLs) or MPCs of the disclosure may be generated by (i) differentiating PSCs using a media supplemented with hematopoietic differentiation factors; and (ii) differentiating the CD43+ iHPCs into iMGLs or MPCs using a microglial differentiating media.

In other aspects, the human microglial-like cells (iMGLs) or MPCs of the disclosure may be produced from a cell of a first type comprising the steps of: (i) differentiating a cell of a first type into an iHPC; and (ii) differentiating the iHPC to produce an iMGL or MPC. In some aspects, the cell of a first type is not a PSC or an ESC.

In some aspects, the PSCs are not derived from embryoid bodies. In some aspects, the PSCs include single-cell PSCs. In some aspects, the PSCs are not CD43+ before differentiation. In some aspects, the PSCs are not CD34+ before differentiation. In some aspects, the PSCs are not CD31+. In some aspects, the PSCs are not CD45+ before differentiation.

In some aspects, the PSCs are or include induced PSCs (iPSCs). In some aspects, the PSCs are or include embryonic stem cells (ESCs). In some aspects, the PSCs are mammalian PSCs. In some aspects, the PSCs are human PSCs. In some aspects, the PSCs are mouse PSCs.

Incubation Period for Differentiating PSCs or iPSCs into HPCs

The incubation periods instantly provided describe the length of the incubation period for differentiating the isolated cell into an HPC. In some embodiments, the differentiating comprises incubating PSCs or iPSCs using the differentiation media supplemented with hematopoietic differentiation factors. In some aspects, differentiating PSCs to produce iHPCs comprises an incubation period that is between about 5 and 15 days. For example, the incubation period may be about 5 days, 6 days, 7 days, 8 days, 9 days, 10 days, 11 days, 12 days, 13 days, 14 days, 15 days, 16 days, 17 days. 18 days, 19 days, 20 days, 21 days, 22 days, 23 days, 24 days, 25 days, 26 days, 27 days, or 28 days. In some aspects, the incubation period is about 7 days. In some aspects, the incubation period is about 14 days. In some aspects, the incubation period is about 21 days. In some aspects, the differentiation period is up to 28 days. In some aspects, the differentiation period is over 28 days. In some aspects, the differentiation period is less than 28 days.

Incubation Period for Differentiating HPCs into iMGLs or MPCs

The incubation periods instantly provided describe the length of the incubation period for differentiating the isolated cell into an iMGL or MPC. In some aspects, the incubation period is less than about 3 days. In some embodiments, the differentiating occurs over a period of at least 14 days in vitro. In some embodiments, the differentiating occurs over a period of about 14 days in vitro. In some embodiments, the differentiating occurs over a period of at least about 7 days in vitro. In some embodiments, the differentiating occurs over a period of about 7 days in vitro. In some embodiments, the differentiating occurs over a period of at least about 1 day in vitro. In some embodiments, the incubating occurs over a period of at least about 1 day. In some embodiments, the incubating occurs over a period of at least about 1 day, 2 days, 3 days, 4 days, 5 days, 6 days, 7 days, 8 days, 9 days, 10 days, 12 days, 14 days, 15 days, 16 days, 17 days, 18 days, 19 days, 20 days, 21 days, 22 days, 23 days, 24 days, 25 days, 26 days, 27 days, 28 days in vitro. In some embodiments, the differentiating occurs over a period of no more than 28 days in vitro. In some embodiments, the differentiating occurs over a period of no more than 14 days in vitro. In some embodiments, the differentiating occurs over a period of no more than 7 days in vitro. In some embodiments, the differentiating occurs over a period of no more than 5 days in vitro. In some embodiments, the differentiating occurs over a period of no more than about 28 days, 25 days, 21 days, 20 days, 18 days, 17 days, 16 days, 15 days, 14 days, 13 days, 12 days, 11 days, 10 days, 9 days, 8 days, 7 days, 6 days, 5 days in vitro. In some embodiments, the differentiating occurs over a period of 1-7 days. In some embodiments, the differentiating occurs over a period of 1-14 days. In some embodiments, the differentiating occurs over a period of about 1-21 days. In some embodiments, the differentiating occurs over a period of about 1-28 days. In some embodiments, the differentiating occurs over a period of about 7-14 days. In some embodiments, the differentiating occurs over a period of about 7-21 days. In some embodiments, the differentiating occurs over a period of about 7-28 days.

Cell Culture Medium for Differentiating HPCs into iMGLs or MPCs

In some aspects, the media used to differentiate the iHPCs into iMGLs or MPCs comprises any one or combination of the factors CSF-1, IL-34, and TGFβ1. In some aspects, the media comprises all of the factors CSF-1, IL34, and TGFβ1. In some aspects, the concentration of the CSF1 in the media is between 5 ng/ml and 50 ng/ml. In some aspects, the concentration of the CSF1 in the media is between 15 ng/ml and 35 ng/ml or between 20 ng/ml and 30 ng/ml. In some aspects, the concentration of CSF1 in the media is 25 ng/ml. In some aspects, the concentration of the IL-34 in the media is between 25 ng/ml and 125 ng/ml. In some aspects, the concentration of the IL-34 in the media is between 80 ng/ml and 120 ng/ml or between 90 ng/ml and 110 ng/ml. In some aspects, the concentration of IL34 in the media is 100 ng/ml. In some aspects, the concentration of the TFGβ1 in the media is between 2.5 ng/ml and 100 ng/ml. In some aspects, the concentration of the TFGβ1 in the media is between 30 ng/ml and 70 ng/ml or between 40 ng/ml and 60 ng/ml. In some aspects, the concentration of TGFβ1 in the media is 50 ng/ml. Some aspects relate to a medium comprising any one or a combination of the factors CSF1, IL-34, and TGFβ1.

In some aspects, the media used to differentiate the iHPCs into iMGLs or MPCs comprises TFGβ2. In some aspects, the concentration of the TFGβ2 in the media is between 2.5 ng/ml and 100 ng/ml. In some aspects, the concentration of the TFGβ2 in the media is between 30 ng/ml and 70 ng/ml or between 40 ng/ml and 60 ng/ml. In some aspects, the concentration of TGFβ2 in the media is 50 ng/ml.

In some aspects, the media used to differentiate the iHPCs into iMGLs or MPCs comprises a TFGβ mimetic. Examples of TGFβ mimetics include IDE1 and IDE2. In some aspects, the TFGβ mimetic has one or more off-target effects and/or affects a SOX signaling pathway. In some aspects, the concentration of the TFGβ mimetic in the media is between 2.5 ng/ml and 100 ng/ml. In some aspects, the concentration of the TFGβ mimetic in the media is between 30 ng/ml and 70 ng/ml or between 40 ng/ml and 60 ng/ml. In some aspects, the TGFβ mimetic activates a TGFβ signaling pathway.

In some embodiments, the cell culture media of the second incubation period wherein the cell culture media for the second incubation period comprises: IL-34, CSF-1, and TGFβ1. In some embodiments, the cell culture media of the second incubation period wherein the cell culture media for the second incubation period comprises: IL-34, CSF-1, and TGFβ2. In some embodiments, the cell culture media of the second incubation period wherein the cell culture media for the second incubation period comprises: IL-34, CSF-1, and a TGFβ mimetic.

In some aspects, the media used to differentiate iHPCs into iMGLs or MPCs is a serum-free media.

Purity of Edited and Differentiated iMGLs, MPCs, or HPCs

Assessing the purity of the engineered and edited iMGLs, MPCs, or HPCs generated according to the method provided herein may be accomplished through utilization of any method known in the art for determining the purity of microglial cells or microglial progenitor cells. In some aspects, the purity levels are assessed by the expression and/or colocalization of the factors P2RY12 and TREM2. In some aspects, the purity levels are assessed by the expression and/or colocalization of Trem2, Iba1, and/or Pu1. In some embodiments, the purity levels are assessed by the expression of CD11b. In some embodiments, the purity levels are assessed by the expression of any canonical microglial markers described above.

In some aspects, the engineered and edited iMGLs or MPCs produced using the methods described herein results in a pure population of iMGLs or MPCs that is between 70% pure and 100% pure. In some aspects, the iMGLs or MPCs produced using the methods described herein results in a pure population of iMGLs or MPCs that is between 80% pure and 100% pure. In some embodiments, the iMGLs or MPCs produced using the methods described herein are at least 95% pure. For example, the population of iMGLs or MPCs will be 80% pure, 81% pure, 82% pure, 83% pure, 84% pure, 85% pure, 86% pure. 87% pure, 88% pure, 89% pure, 90% pure, 91% pure, 92% pure, 93% pure, 94% pure, 95% pure, or 96% pure, 97% pure, 98% pure, 99% pure, or 100% pure. In some aspects, the population of iMGLs or MPCs produced is greater than 96% pure.

Methods for Detecting Differentiation of HfPCs, MPCs, and iMGLs

The edited and differentiated iMGLs, MPCs, or HPCs produced by any of the methods described herein may express any factor or any combination of factors that a typical canonical microglial cell expresses. In some embodiments, the edited and differentiated iMGLs, MPCs, or HPCs express the synthetic CSF1R gene described herein. In some embodiments, the edited and differentiated iMGLs, MPCs, or HPCs express the repaired CSF1R gene described above. In some aspects, the iMGLs produced are ckit−/CD45+. In some aspects, the ckit−/CD45+ iMGLs are detected using flow cytometry, immunofluorescence microscopy, qPCR, RNAseq, or proteomics. In some aspects, other cell types are detected using flow cytometry, immunofluorescence microscopy, qPCR, RNAseq, or proteomics. In some aspects, the iMGLs produced comprise two separate populations of iMGLs: (1) CD45+/CX3CR1—and (2) CD45+/CX3CR1+. In some aspects, the iMGLs produced are CD43+, CD235a+, or CD41+. In some aspects, the iMGLs produced are CD43+/CD235a+/CD41+.

Any of the methods for producing iMGLs or MPCs described herein may result in a differentiation step of the CD43+ iHPCs in which there is a commitment of cells to a microglial lineage early during the differentiation process. In some aspects, iMGLs that are ckit−/CD45+ are detected on day 14 of the incubation period used for differentiating CD43+ iHPCs into iMGLs or MPCs. Determining whether there is a commitment to an iMGL lineage may be done through testing for expression of any factors that are known to be markers for cells that are committed to a microglia fate. In some aspects, determining whether the cells are committed to an iMGL lineage is determined through assessing expression of the transcription factor PU.1 and/or the microglia-enriched protein Trem2. In some aspects, the cell markers may be detected using flow cytometry, immunofluorescence microscopy, qPCR, RNAseq, or proteinomics.

In some aspects, a method of producing iMGLs or MPCs from induced PSCs is provided that comprises the steps: (i) differentiating PSCs into induced hematopoietic progenitor cells (iHPCs) and (ii) differentiating iHPCs to produce iMGLs or MPCs. In some aspects, this method further comprises step (iii) of maturing the iMGLs produced from step (ii). In some aspects, the PSCs include induced PSCs (iPSCs) or embryonic stem cells (ESCs). In some aspects, the PSCs are mammalian PSCs, such as from a human or a mouse.

One aspect of the disclosure provides a method of preparing a therapeutic composition, said method comprising: introducing, to a CSF1R gene of an isolated human cell (e.g., iPSC, HPC) having a mutation in CSF1R, a CSF1R coding sequence (CDS) 3′ of a fms-intronic regulatory element (FIRE) and 5′ of the mutation in the CSF1R gene, wherein the CDS comprises a polyadenylation signal to prevent transcription of the mutation in the CSF1R gene, wherein the CDS encodes wildtype CSF1R. The step of introducing the CDS may comprise the introducing to the cell: CRISPR endonuclease, e.g., Cas9, a guide RNA (gRNA), and a polynucleotide comprising a CSF1R cDNA fragment. In some embodiments, the CDS comprises a silent mutation effective for preventing enzyme-mediated DNA cleavage, e.g.. Cas9 cleavage. The method may further comprise incubating the isolated cell in a culture media comprising a differentiation factor effective for differentiating the isolated human cell.

One aspect of the disclosure provides a method of preparing a therapeutic composition, said method comprising: introducing, to a CSF1R gene of an isolated human cell (e.g., iPSC, HPC) having a mutation in CSF1R, a CSF1R coding sequence (CDS) into Exon 2 and 3′ of a fms-intronic regulatory element (FIRE), wherein the CDS comprises a polyadenylation signal to prevent transcription of Exons 3-22, wherein the CDS encodes wildtype CSF1R. The step of introducing the CDS may comprise the introducing to the cell: CRISPR endonuclease, e.g., Cas9, a guide RNA (gRNA), and a polynucleotide comprising a CSF1R cDNA fragment. In some embodiments, the CDS comprises a silent mutation effective for preventing enzyme-mediated DNA cleavage, e.g., Cas9 cleavage. The method may further comprise incubating the isolated cell in a culture media comprising a differentiation factor effective for differentiating the isolated human cell.

One aspect of the disclosure provides a method of preparing a therapeutic composition, said method comprising: repairing a mutation in a CSF1R gene in an isolated human cell (e.g., iPSC, HPC) such that the isolated human cell expresses wildtype CSF1R (e.g., human CSF1R). The method may further comprise incubating the isolated cell in a culture media comprising a differentiation factor effective for differentiating the isolated human cell. In some embodiments, the step of repairing the mutation comprises introducing to the cell a CRISPR endonuclease, e.g., Cas9, a guide RNA sequence (gRNA) complementary to a nucleotide sequence adjacent to the nucleotide sequence encoding the mutation in the CSF1R gene, and a homology-directed repair template, e.g., the wildtype CSF1Ra sequence at the position in the CSF1R gene corresponding to the position of the mutation. In some embodiments, the homology-directed repair template is a single-stranded oligodeoxynucleotide (ssODN)

Referring to the disclosures herein, including the embodiments above, the mutation in the CSF1R gene may be one associated with a leukodystrophy, e.g., Adult-Onset Leukodystrophy (ALSP).

One aspect of the disclosure provides a method of populating a microglial niche in the brain of a subject in need thereof, comprising: administering a therapeutic composition to the brain of the subject, the therapeutic composition comprising a pharmaceutical carrier; and an engineered iPSC-derived microglia (e.g., human) or iPSC-derived HPC (e.g., human) comprising a Colony Stimulating Factor 1 Receptor (CSF1R) gene, wherein the CSF1R gene comprises a CSF1R coding sequence (CDS) inserted 3′ of a fms-intronic regulatory element (FIRE) and 5′ of a mutation in the CSF1R gene, wherein the CDS comprises a polyadenylation signal to prevent transcription of the mutation in the CSF1R gene, wherein the CDS encodes wildtype CSF1R (e.g., human CSF1R). In some embodiments, the CDS comprises a silent mutation effective for preventing enzyme-mediated DNA cleavage, wherein the engineered cells differentiate into microglia in vivo.

### Editing Myeloid Cells

Provided herein is a method of editing a mutant CSF1R gene in an isolated myeloid cell comprising: repairing a point mutation in the mutant CSF1R gene, or inserting a CSF1R coding sequence 3′ of a FIRE in the mutant CSF1R gene, thereby repairing or replacing the mutant CSF1R gene in the myeloid cell, according to any of the gene editing strategies for repairing a point mutation in a defective gene of inserting a synthetic CSF1R allele as described above.

In some embodiments, the mutant CSF1R gene is associated with a leukodystrophy, such as, for example, the defective CSF1R gene or any point mutation in the defective CSF1R as described above. In some embodiments, the mutant CSF1R gene is associated with Adult-onset leukoencephalopathy (ALSP). In some embodiments, the myeloid cell is an iPSC, HSC, HPC, or iMGL. In some embodiments, the myeloid cell was derived from an iPSC, HSC or HPC. In some embodiments, a sample comprising the isolated myeloid cell was collected from a donor by any of the methods described herein. As nonlimiting examples, the donor sample can comprise fibroblasts, stem cells, or pluripotent stem cells. In some embodiments, the sample comprises a skin sample, a bone marrow sample, a blood sample, or a cerebrospinal fluid sample. In some embodiments, the isolated cell was collected from a donor, wherein the donor will receive the isolated cell after the inserting or repairing of the mutant CSF1R gene in the isolated myeloid cell. In some embodiments, the point mutation is in a sequence encoding a kinase domain of a CSF1R polypeptide. In some embodiments, the editing comprises contacting the mutant CSF1R gene with a TALEN, a zinc-finger endonuclease, a meganuclease, or any of the gene editing methods described above, most preferably a CRISPR Cas endonuclease. In some embodiments, the editing comprises contacting the defective human CSF1R gene with a CRISPR endonuclease. In some embodiments, the CRISPR endonuclease comprises Cas9. In some embodiments, the CRISPR/Cas gene editing comprises a Cas9 endonuclease, a catalytically dead Cas9 endonuclease, or a nickase Cas9 endonuclease. In some embodiments, CRISPR/Cas gene editing comprises a M-MLV reverse transcriptase (RT) fused to a nickase Cas9 endonuclease. In some embodiments, the repairing comprises repairing the point mutation by introducing into the isolated myeloid cell: (i) a Cas9 polypeptide or a polynucleotide encoding the Cas9 polypeptide, (ii) a gRNA complementary to a nucleotide sequence adjacent to the point mutation, and (iii) a homology-directed repair template polynucleotide comprising a wildtype CSF1R sequence at a position in the CSF1R gene corresponding to the position of the point mutation. In some embodiments, the homology directed repair template polynucleotide is a single-stranded DNA oligonucleotide. In some embodiments, the repairing further comprises introducing a pegRNA into the cell. In some embodiments, the repairing further comprises introducing into the isolated myeloid cell: a catalytically dead Cas9 endonuclease, or a nickase Cas9 endonuclease. In some embodiments, CRISPR/Cas gene editing comprises a M-MLV reverse transcriptase (RT) fused to a nickase Cas9 endonuclease.

### CSF1R Mutant Repairing Strategy in Myeloid Cells

In another aspect, the point mutation in the defective CSF1R gene may be repaired to eliminate a point mutation, preferably a disease-associated CSF1R point mutation, including but not limited to any of the disease-associated point mutations described above (e.g., M875, L786, M875, N854, G589, M8751, L786S, M875T, N854K, G589E, G481, P132L, Y540, K627, H643, H643Q, T833, T833M, etc.), in the genome of the isolated myeloid cell.

In some embodiments, the point mutation comprises the M875I mutation of this disclosure. As disclosed herein the M875I point mutation may be corrected, in some embodiments, with the gRNA comprising the nucleic acid sequence of SEQ ID NO: 12, or alternatively, a gRNA comprising a nucleic acid sequence having at least 90% sequence identity to the nucleic acid sequence of SEQ ID NO: 12. In some embodiments, the M875I point mutation may be corrected when the repairing comprises the homology-directed repair template polynucleotide comprising the nucleic acid sequence of SEQ ID NO: 7. In some embodiments, the M875I point mutation may be corrected when the repairing comprises the homology-directed repair template polynucleotide comprising at least 90% or 95% sequence identity to the nucleic acid sequence of SEQ ID NO: 7. In some embodiments, the M8751 point mutation may be corrected when the repairing comprises introducing into the cell: (i) a gRNA comprising the nucleic acid sequence of SEQ ID NO: 12, and (ii) the homology-directed repair template polynucleotide comprising the nucleic acid sequence of SEQ ID NO: 7.

In some embodiments, the point mutation comprises the L786S mutation of this disclosure. As disclosed herein the L786S point mutation may be corrected, in some embodiments, with the gRNA comprising the nucleic acid sequence of SEQ ID NO: 13, or alternatively, a gRNA comprising a nucleic acid sequence having at least 90% sequence identity to the nucleic acid sequence of SEQ ID NO: 13. In some embodiments, the L786S point mutation may be corrected when the repairing comprises the homology-directed repair template polynucleotide comprising the nucleic acid sequence of SEQ ID NO: 8. In some embodiments, the L786S point mutation may be corrected when the repairing comprises the homology-directed repair template polynucleotide comprising at least 90% or 95% sequence identity to the nucleic acid sequence of SEQ ID NO: 8. In some embodiments, the L786S point mutation may be corrected when the repairing comprises introducing into the cell: (i) a gRNA comprising the nucleic acid sequence of SEQ ID NO: 13, and (ii) the homology-directed repair template polynucleotide comprising the nucleic acid sequence of SEQ ID NO: 8.

In some embodiments, the point mutation comprises the M875T mutation of this disclosure. As disclosed herein the M875T point mutation may be corrected, in some embodiments, with the gRNA comprising the nucleic acid sequence of SEQ ID NO: 14, or alternatively, a gRNA comprising a nucleic acid sequence with at least 90% sequence identity to the nucleic acid sequence of SEQ ID NO: 14. In some embodiments, the M875T point mutation may be corrected when the repairing comprises the homology-directed repair template polynucleotide comprising the nucleic acid sequence of SEQ ID NO: 9. In some embodiments, the M875T point mutation may be corrected when the repairing comprises the homology-directed repair template polynucleotide comprising at least 90% or 95% sequence identity to the nucleic acid sequence of SEQ ID NO: 9. In some embodiments, the M875T point mutation may be corrected when the repairing comprises introducing into the cell: (i) a gRNA comprising the nucleic acid sequence of SEQ ID NO: 14, and (ii) the homology-directed repair template polynucleotide comprising the nucleic acid sequence of SEQ ID NO: 9.

In some embodiments, the point mutation comprises the N854K mutation of this disclosure. As disclosed herein the N854K point mutation may be corrected, in some embodiments, with the gRNA comprising the nucleic acid sequence of SEQ ID NO: 15, or alternatively, a gRNA comprising a nucleic acid sequence with at least 90% sequence identity to the nucleic acid sequence of SEQ ID NO: 15. In some embodiments, the N854K point mutation may be corrected when the repairing comprises the homology-directed repair template polynucleotide comprising the nucleic acid sequence of SEQ ID NO: 10. In some embodiments, the N854K point mutation may be corrected when the repairing comprises the homology-directed repair template polynucleotide comprising at least 90% or 95% sequence identity to the nucleic acid sequence of SEQ ID NO: 10. In some embodiments, the N854K point mutation may be corrected when the repairing comprises introducing into the cell: (i) a gRNA comprising the nucleic acid sequence of SEQ ID NO: 15, and (ii) the homology-directed repair template polynucleotide comprising the nucleic acid sequence of SEQ ID NO: 10.

In some embodiments, the point mutation comprises the G589E mutation of this disclosure. As disclosed herein the G589E point mutation may be corrected, in some embodiments, with the gRNA comprising the nucleic acid sequence of SEQ ID NO: 16, or alternatively, a gRNA comprising a nucleic acid sequence with at least 90% sequence identity to the nucleic acid sequence of SEQ ID NO: 16. In some embodiments, the G589E point mutation may be corrected when the repairing comprises the homology-directed repair template polynucleotide comprising the nucleic acid sequence of SEQ ID NO: 11. In some embodiments, the G589E point mutation may be corrected when the repairing comprises the homology-directed repair template polynucleotide comprising at least 90% or 95% sequence identity to the nucleic acid sequence of SEQ ID NO: 11. In some embodiments, the G589E point mutation may be corrected when the repairing comprises introducing into the cell: (i) a gRNA comprising the nucleic acid sequence of SEQ ID NO: 16, and (ii) the homology-directed repair template polynucleotide comprising the nucleic acid sequence of SEQ ID NO: 11.

### CSF1R Insertion Strategy in Myeloid Cells

In some instances, the isolated myeloid cell is or can be edited to express the synthetic CSF1R gene described herein. In some embodiments, the isolated myeloid cell is edited to harbor the inserted polynucleotide comprising a CSF1R coding sequence (e.g., synthetic CSF1R gene or CSF1R cDNA fragment) described above. In some embodiments, the inserting comprises introducing into the cell: (i) a Cas9 polypeptide or a polynucleotide encoding the Cas9 polypeptide, (ii) a gRNA comprising SEQ ID NO: 5 or a polynucleotide encoding the gRNA, and (iii) a polynucleotide comprising a CSF1R coding sequence. In some embodiments, wherein the polynucleotide further comprises a stop codon and a poly-A signal 3′ of the CSF1R coding sequence. In some embodiments, wherein the polynucleotide comprising a CSF1R coding sequence further comprises a silent mutation (e.g., adenosine, thymine, or cystine as a substitution for guanidine at the fifth nucleotide position of the nucleotide sequence of SEQ ID NO: 2). In some instances, the isolated myeloid cell harbors the synthetic gene comprises the silent mutation described above. In some embodiments, the CSF1R coding sequence encodes a CSF1R polypeptide having at least 80% sequence identity to SEQ ID NO: 1. In some embodiments, the synthetic CSF1R gene comprises the nucleic acid sequence of SEQ ID NO: 2. In some embodiments, the synthetic CSF1R gene comprises a nucleic acid sequence having at least 70%, 75%, 80%, 85%, 87%. 90%, 92%, 95%, 96%, 97%, 98%, or 99% sequence identity to the nucleotide sequence of SEQ ID NO: 2. In some embodiments, the polynucleotide comprising the CSF1R coding sequence comprises a nucleic acid sequence having at least 90% sequence identity to the nucleotide sequence of SEQ ID NO: 2. In some embodiments, the synthetic CSF1R gene comprises a nucleic acid sequence having at least 97% sequence identity to the nucleotide sequence of SEQ ID NO: 2. In some embodiments, the synthetic CSF1R gene consists of the nucleic acid sequence of SEQ ID NO: 2.

In one aspect, the synthetic gene described herein is introduced into the isolated myeloid cell by editing a target gene (e.g., defective CSF1R gene) to insert the polynucleotide comprising a CSF1R coding sequence encodes a protein, for example but not limited to, a human CSF1R protein, capable of normal wildtype CSF1R protein expression level and CSF1R protein function (e.g., CSF1R tyrosine receptor kinase activity). The editing of the mutant CSF1R gene (e.g., defective CSF1R gene), as described above, may require inserting the polynucleotide comprising a CSF1R coding sequence into the CSF1R gene locus of an isolated myeloid cell (e.g., iPSC, PSC, HPC, HSC). In such cases, the polynucleotide comprising a CSF1R coding sequence may be inserted between the left homology arm and aright homology arm flanking a portion of the defective CSF1R gene locus located 3′ of the fms-intronic response element (FIRE) of the CSF1R. In some cases, the left homology arm may have a nucleic acid sequence having a sequence identity of at least 80%, 90%, 95%, 97%, or 99% to the nucleic acid sequence of SEQ ID NO: 3. In some cases, the left homology arm may consist of the nucleic acid sequence of SEQ ID NO: 3. In some cases, the right homology arm may have a nucleic acid sequence having a sequence identity of at least 80%, 90%, 95%, 97%, or 99% to the nucleic acid sequence of SEQ ID NO: 4. In some cases, the right homology arm may consist of the nucleic acid sequence of SEQ ID NO: 4.

In some embodiments, the editing comprises introducing into the cell: a Cas9 polypeptide or a polynucleotide encoding the Cas9 polypeptide, a gRNA comprising a sequence of the CSF1R gene or a polynucleotide encoding the gRNA, and a polynucleotide comprising a polynucleotide comprising a CSF1R coding sequence, thereby replacing the CSF1R gene. In some embodiments, the gRNA comprises the nucleic acid sequence of SEQ ID NO: 5. In some embodiments, the gRNA comprises a nucleic acid sequence with at least 90% sequence identity to the nucleic acid sequence of SEQ ID NO: 5.

### Function of the Repaired or Inserted CSF1R Gene in Myeloid Cells

The expression of the repaired CSF1R gene described herein can result in essentially normal CSF1R gene expression and function in any of the edited myeloid cell types, which is also increased in comparison to the unmodified isolated source cell (e.g., iPSC, PSC, MPC, HPC, HSC) harboring at least one allele of the defective CSF1R gene described above. It shall be understood that the edited myeloid cells generated by the method described herein can have the any of the consequences on CSF1R gene expression, CSF1R receptor tyrosine kinase activity, microglia function, or effects of transplantation of the edited and engineered cells provided herein.

In some embodiments, a transcriptional profile of microglia-related genes of the edited myeloid cell harboring the inserted or repaired CSF1R gene described herein (e.g., synthetic CSF1R gene) more closely resembles a transcriptional profile of the microglia-related genes in a positive control MC-derived iMGL with exactly two native, wildtype CSF1R alleles compared to a transcriptomic profile of the microglia-related genes in an otherwise identical negative control iMGL comprising the defective CSF1R gene.

In some embodiments, CSF1R expression in the edited myeloid cell is at least 80% of a CSF1R expression level in an otherwise identical control cell having two native, wildtype CSF1R alleles. In some embodiments, CSF1R expression in the edited myeloid cell is at least 60%, 70%, 75%, 80%, 85%, 90%, 95%, or 100% of a CSF1R expression level in an otherwise identical control cell having two native, wildtype CSF1R alleles.

In some embodiments, CSF1R tyrosine receptor kinase activity in the edited myeloid cell is at least 80% of CSF1R tyrosine receptor kinase activity in an otherwise identical control cell having two native, wildtype CSF1R alleles. CSF1R tyrosine receptor kinase activity in the edited myeloid cell is at least 60%, 70%, 75%, 80%, 85%, 90%, 95%, or 100% of CSF1R tyrosine receptor kinase activity in an otherwise identical control cell having two native, wildtype CSF1R alleles.

### Transplantation of Engineered Cells

Any and all of the edited cells described herein (e.g., iMGLs, MPC, HPCs, MCs, etc.) may be transplanted into a subject in need thereof by any suitable means. In one aspect, transplantation of the edited and/or differentiated cells described herein may be a cell therapy to restore microglial, normal brain function, and/or normal homeostasis to the subject. In some instances, the composition may be effective for replacing a portion of microglia in the brain of a subject.

Edited cells generated according to the methods provided herein may be administered according to any means of administering the edited cells described below. As nonlimiting examples, the edited cells may be administered to the subject for transplantation into the brain of the subject via intramuscular, intranasal, stereotactic, subretinal, or, more preferably, via intracranial or intravenous injection of the edited cells. In an animal body, it can prove advantageous to apply the pharmaceutical compositions as described above via an intravenous injection or by direct injection into the target tissues. For systemic application, the intravenous, intravascular, intramuscular, intranasal, stereotactic, intraparenchymal, intracerebroventricular, subretinal or intrathecal routes are preferred. A more local application may be affected subcutaneously, intradermally, intracutaneously, intralobally, intramedullarly, or directly in or near the tissue to be treated.

Period after Transplantation

The cell therapeutic effects resultant of the transplanting of the edited cells of this disclosure may be assessed after a period of time since the transplanting was performed. In some embodiments, a period of at least about 1 week, 2 weeks, 3 weeks, 4 weeks, 5 month, 6 weeks, 7 weeks, 8 weeks, 9 weeks, 10 weeks, 11 weeks, 12 weeks, 4 months, 5 months, 6 months, 7 months, 8 months, 10 months, 1 year, 1.5 years, 2 years, 2.5 years, 3 years, 4 years, or 5 years has passed since the transplanting of the edited cells provided herein. In some embodiments, a period of at least about 2 weeks, 3 weeks, 4 weeks, 5 month, 6 weeks, 7 weeks, 8 weeks, 9 weeks, 10 weeks, 11 weeks, or 12 weeks has passed since the transplanting of the edited cells provided herein. In some embodiments, a period of at least about 4 weeks has passed since the transplanting of the edited cells provided herein. In some embodiments, a period of at least about 6 weeks has passed since the transplanting of the edited cells provided herein. In some embodiments, a period of at least about 8 weeks has passed since the transplanting of the edited cells provided herein. In some embodiments, a period of at least about 12 weeks has passed since the transplanting of the edited cells provided herein. In some embodiments, a period of no more than about 2 weeks, 3 weeks, 4 weeks, 5 month, 6 weeks, 7 weeks, 8 weeks, 9 weeks, 10 weeks, 11 weeks, 12 weeks, 4 months, 5 months, 6 months, 7 months, 8 months, 10 months, or 1 year has passed since the transplanting of the edited cells provided herein. In some embodiments, a period of about 4 weeks has passed since the transplanting of the edited cells provided herein. In some embodiments, a period of about 6 weeks has passed since the transplanting of the edited cells provided herein. In some embodiments, a period of about 8 weeks has passed since the transplanting of the edited cells provided herein. In some embodiments, a period of about 12 weeks has passed since the transplanting of the edited cells provided herein.

### Minimum Number of Cells

In some embodiments, the transplanting comprises administering at least about 2.5×10⁵ edited cells of this disclosure by any route of administration described herein. In some embodiments, the transplanting comprises administering about 5 ×10⁵ edited cells of this disclosure by any route of administration described herein. In some embodiments, the transplanting comprises administering at least about 2.5×10⁵ edited cells of this disclosure via an intracranial injection. In some embodiments, the transplanting comprises administering about 5×10⁵ edited cells of this disclosure via an intracranial injection.

### Effects of Transplanting the Edited Cells

Provided herein is a method of transplanting the edited iMGL, MPC, HPC, MCs, or microglial precursor cell (generically and collectively referred to as “edited cells” henceforth) into a brain of a subject. In some embodiments, the transplanting of the edited cells comprises increasing the number or density of Iba1-expressing edited microglia in the brain of the subject after a time period as described above has passed since the transplantation of the edited iMGL, HPC, microglial precursor cell, or myeloid cell into the subject. In some embodiments, the transplanting of the edited cells comprises increasing the number, density, or average size of excitatory synapses in the brain of the subject, after a time period as described above has passed since the transplantation of the edited iMGL, HPC, microglial precursor cell, or myeloid cell into the subject. In some embodiments, the transplanting of the edited cells comprises increasing the PSD95 or NSE expression in the brain of the subject, after a time period as described above has passed since the transplantation of the edited iMGL, HPC, microglial precursor cell, or myeloid cell into the subject. In some embodiments, the transplanting of the edited cells comprises decreasing the accumulation of secreted osteopontin (OPN) in the brain of the subject, after a time period as described above has passed since the transplantation of the edited iMGL, HPC, microglial precursor cell, or myeloid cell into the subject. In some embodiments, the transplanting of the edited cells comprises decreasing the frequency of axonal spheroids immunoreactive for neurofilament SMI312 and LAMP1 in the brain of the subject, after a time period as described above has passed since the transplantation of the edited iMGL, HPC, microglial precursor cell, or myeloid cell into the subject. In some embodiments, the transplanting of the edited cells comprises decreasing the number, density, or frequency of hydroxyapatite calcium crystals in the brain of the subject, after a time period as described above has passed since the transplantation of the edited iMGL, HPC, microglial progenitor cell, or myeloid cell into the subject. In some embodiments, the transplanting of the edited cells comprises decreasing the levels of Tau phosphorylated at Thr217 (pTau217) in the brain, cerebrospinal fluid, or blood plasma of the subject, after a time period as described above has passed since the transplantation of the edited iMGL, HPC, microglial precursor cell, or myeloid cell into the subject. In some embodiments, the transplanting of the edited cells comprises decreasing the GFAP expression levels in the brain, cerebrospinal fluid, or blood plasma of the subject, after a time period as described above has passed since the transplantation of the edited iMGL, HPC, microglial precursor cell, or myeloid cell into the subject. In some embodiments, the transplanting of the edited cells comprises decreasing the MCP-1 expression levels in the brain of the subject, after a time period as described above has passed since the transplantation of the edited iMGL, HPC, microglial precursor cell, or myeloid cell into the subject. In some embodiments, the transplanting of the edited cells comprises decreasing in the brain of the subject of the expression levels any one of: SERPINA3N, neurofilament light chain (NFL), GFAP, pTau217, MCP-1 (CCL2), Osteopontin (SPP1), LAMP1, or LAMP2 genes, after a time period as described above has passed since the transplantation of the edited iMGL, HPC, microglial precursor cell, or myeloid cell into the subject. In some embodiments, the transplanting of the edited cells comprises decreasing in the brain of the subject of the expression levels at least two of SERPINA3N, neurofilament light chain (NFL), GFAP, pTau217, MCP-1 (CCL2), Osteopontin (SPP1), LAMP1, or LAMP2 genes, after a time period as described above has passed since the transplantation of the edited iMGL, HPC, microglial precursor cell, or myeloid cell into the subject. In some embodiments, the transplanting of the edited cells comprises decreasing in the brain of the subject of the expression levels of at least three of: SERPINA3N, neurofilament light chain (NFL), GFAP, pTau217, MCP-1 (CCL2), Osteopontin (SPP1), LAMP1, or LAMP2 genes, after a time period as described above has passed since the transplantation of the edited iMGL, HPC, microglial precursor cell, or myeloid cell into the subject. In some embodiments, the gene expression levels of SERPINA3N, neurofilament light chain (NFL), GFAP, pTau217, MCP-1 (CCL2), Osteopontin (SPP1), LAMP1, or LAMP2 genes can be decreased by at least 20% after a time period as described above has passed since the transplantation of the edited iMGL, HPC, microglial precursor cell, or myeloid cell into the subject. In some embodiments, the gene expression levels of SERPINA3N, neurofilament light chain (NFL), GFAP, pTau217, MCP-1 (CCL2), Osteopontin (SPP1), LAMP1, or LAMP2 genes can be decreased by at least 20%, 30%, 40%, 50%, 60%, 70%, or 80% after a time period as described above has passed since the transplantation of the edited iMGL, HPC, microglial precursor cell, or myeloid cell into the subject.

In some embodiments, the transplanting of the edited cells comprises any two of: increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SMI312 and LAMP1 in the brain of the subject; decreasing the number, density, or frequency of hydroxyapatite calcium crystals in the brain of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; or decreasing the MCP-1 expression levels in the brain of the subject, as described above has passed since the transplantation of the edited iMGL, HPC, microglial precursor cell, or myeloid cell into the subject after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

In some embodiments, the transplanting of the edited cells comprises any three of: increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SMI312 and LAMP1 in the brain of the subject; decreasing the number, density, or frequency of hydroxyapatite calcium crystals in the brain of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; or decreasing the MCP-1 expression levels in the brain of the subject, as described above has passed since the transplantation of the edited iMGL, HPC, microglial precursor cell, or myeloid cell into the subject after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

In some embodiments, the transplanting of the edited cells comprises increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SM1312 and LAMP1 in the brain of the subject; decreasing the number, density, or frequency of hydroxyapatite calcium crystals in the brain of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; and decreasing the MCP-1 expression levels in the brain of the subject, after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

### Donor Sources of Cells for Transplantation

In various aspects, a sample of cells may be collected from the subject using techniques known in the art, such as biopsy, or the like. In some aspects, the sample of cells may include somatic cells, including blood or skin fibroblasts, which may be collected for iPSC generation. In various aspects, the sample of cells may be reprogrammed into iPSCs using techniques known in the art. Suitable reprogramming systems include integrative viral vector transfer systems, integrative nonviral transfer systems, non-integrative viral transfer systems, nonintegrative nonviral transfer systems, and CRISPR systems. In some embodiments, a sample comprising the isolated cell was collected from a donor. In some embodiments, the sample comprises fibroblasts. In some embodiments, the sample comprises stem cells. In some embodiments, the sample comprises pluripotent stem cells. In some embodiments, the sample comprises a skin sample. In some embodiments, the sample comprises a bone marrow sample. In some embodiments, the sample comprises a blood sample or a cerebrospinal fluid sample. In some embodiments, the isolated cell was collected from a donor, wherein the donor will receive the isolated cell after the editing and differentiating of the isolated cell in the collected sample. In some embodiments, the isolated cell may be any of the engineered cells of this disclosure. In various aspects, human microglial-like cells (iMGLs) or microglial progenitor cells may be delivered to a subject for the treatment of a disease associated with a mutation of a microglial gene by collecting a sample of cells from the subject, reprogramming the cells into induced pluripotent stem cells (iPSCs), deriving iMGLs or microglial progenitor cells from the subject-derived iPSCs, and delivering the derived iMGLs or microglial progenitor cells into the central nervous system of the subject. In some aspects, the method includes delivering the derived iMGLs or microglial progenitor cells into the brain or spinal cord of the subject.

In various aspects, iMGLs or microglial progenitor cells of the disclosure may be generated from autologous PSCs from a subject and transplanted into the subject to treat a disease associated with a mutation of a microglial gene. In some aspects, iMGLs or microglial progenitor cells of the disclosure may be generated from autologous PSCs from a subject, and transplanted into the subject to supplement microglia numbers within the central nervous system of the subject. In various aspects, edited MCs of the disclosure may be generated from autologous PSCs from a subject and transplanted into the subject to treat a disease associated with a mutation of a microglial gene.

In various aspects, allogeneic microglia derived from a donor may be transplanted into the subject using delivery methods as disclosed herein. In some aspects, the iMGLs or microglial progenitor cells of the disclosure may be derived from allogeneic PSCs and used to generate donor iMGLs or microglial progenitors for transplantation into the central nervous system (e.g., brain or spinal cord) of a subject suffering from diseases associated with a mutation of a microglial gene. In some aspects, the donor iMGLs or microglial progenitors may be transplanted into the brain and/or spinal cord of a subject.

### Indications and Measurement of Transplantation

Obstacles related to transplantation procedures are well known, especially graft rejection. Therefore, a method of monitoring the transplantation efficiency of the edited cells described herein is highly desirable, especially following neurosurgical transplantation procedures.

Provided herein is a method of monitoring engraftment of an IMGL or microglial precursor cell in a brain of a subject, comprising: (i) transplanting the iMGLs or microglial precursor cells into the brain of a subject with a leukodystrophy, (ii) obtaining a blood sample from the subject, and (iii) measuring an amount of GFAP or pTau217 in the blood sample, wherein a decrease in the amount of GFAP or pTau217 in the blood sample after transplantation indicates successful engraftment of the iMGLs or microglial precursor cells in the brain. In some embodiments, an amount of GFAP or pTau217 in the blood sample is decreased by at least 30% compared to an amount of GFAP or pTau217 in a blood sample from the subject prior to the transplanting. In some embodiments, the amount of GFAP or pTau217 in the blood sample is decreased by at least 10%, 20%, 30%, 40%, 50%, 60%, 70%, or 80% compared to an amount of GFAP or pTau217 in a blood sample from the subject prior to the transplanting.

In some embodiments, the method of monitoring engraftment of an IMGL or microglial precursor cell in a brain of a subject further comprises measuring an amount of IL-34 or CSF-1 in a cerebrospinal fluid sample, wherein a decrease in the amount of IL-34 or CSF-1 in the cerebrospinal fluid sample after transplantation indicates successful engraftment of the iMGLs or microglial precursor cells in the brain. In some embodiments, an amount of IL-34 or CSF-1 in the cerebrospinal fluid sample is decreased by at least 30% compared to an amount of IL-34 or CSF-1 in a cerebrospinal fluid sample from the subject prior to the transplanting. In some embodiments, the amount of IL-34 or CSF-1 in the cerebrospinal fluid sample is decreased by at least 10%, 20%, 30%, 40%, 50%, 60%, 70%, or 80% compared to an amount of IL-34 or CSF-1 in a cerebrospinal fluid sample from the subject prior to the transplanting.

In some embodiments, method of monitoring engraftment of an IMGL or microglial precursor cell in a brain of a subject further comprises measuring CSF1R levels in the blood sample, wherein an increase in the amount of CSF1R levels in the blood sample after transplantation indicates successful engraftment of the iMGLs or microglial precursor cells in the brain. In some embodiments, an amount of CSF1R levels in the blood sample is increased by at least 20% compared to an amount of CSF1R levels in a blood sample from the subject prior to the transplanting. In some embodiments, the amount of CSF1R levels in the blood sample is increased by at least 10%, 20%, 30%, 40%, 50%, 60%, 70%, or 80% compared to an amount of CSF1R levels in a blood sample from the subject prior to the transplanting.

In some embodiments, the method of monitoring engraftment of an iMGL or microglial precursor cell in a brain of a subject further comprises measuring an amount of complement-4b (C4b) in a blood sample, wherein a decrease in the amount of C4b in the blood sample after transplantation indicates successful engraftment of the iMGLs or microglial precursor cells in the brain. In some embodiments, an amount of C4b in the blood sample is decreased by at least 30% compared to an amount of C4b in a blood sample from the subject prior to the transplanting. In some embodiments, the amount of C4b in the blood sample is decreased by at least 10%, 20%, 30%, 40%, 50%, 60%, 70%, or 80% compared to an amount of C4b in a blood sample from the subject prior to the transplanting.

In some embodiments, method of monitoring engraftment of an IMGL or microglial precursor cell in a brain of a subject further comprises measuring soluble TREM2 (sTREM2) levels in the blood sample, wherein an increase in the amount of sTREM2 levels in the blood sample after transplantation indicates successful engraftment of the iMGLs or microglial precursor cells in the brain. In some embodiments, an amount of sTREM2 levels in the blood sample is increased by at least 20% compared to an amount of sTREM2 levels in a blood sample from the subject prior to the transplanting. In some embodiments, the amount of sTREM2 levels in the blood sample is increased by at least 10%, 20%, 30%, 40%, 50%, 60%, 70%, or 80% compared to an amount of sTREM2 levels in a blood sample from the subject prior to the transplanting.

### Populating a Microglial Niche

The edited and differentiated iMGLs and MPCs of this disclosure are shown herein to engraft, proliferate, and repopulate a microglia-depleted niche in the brain of a subject.

Provided herein is a method of populating a microglial niche in the brain of a subject, the method comprising: obtaining cells with a repaired or replaced gene that corrects a disease-associated mutation, and administering the cells to the brain of the subject, wherein the obtained cells were isolated from the subject or generated by culturing cells that were isolated from the subject, and wherein the administered cells differentiate into microglia in vivo. In some embodiments, the subject was diagnosed with Adult-onset leukoencephalopathy. In some embodiments, wherein the subject is at-risk of developing Adult-onset leukoencephalopathy. In some instances, the subject may be any other subject described below. In some embodiments, the gene is CSF1R. In some embodiments, the subject harbors at least one allele comprising a defective CSF1R gene of this disclosure. In some embodiments, the subject has a CSF1R haploinsufficiency. In some embodiments, the cells are iMGLs, MPCs, or HPCs. In some embodiments, the administering comprises: transplanting the cells into a brain or spinal cord of the subject. In some embodiments, at least 70% of microglia in the brain of the subject are the administered engineered cells.

### Treatment and Cell Therapy for Leukodystrophies

### Leukodystrophies

The present disclosure further relates to compositions comprising effective amounts of the iMGLs or microglial progenitors of the disclosure that may be administered to a subject having a disease associated with a mutation of a CSF1R gene. As described above, mutations in the CSF1R gene underlie leukodystrophies, including ALSP and BANDDOS, which present overlapping clinical manifestations but are caused by autosomal dominant and autosomal recessive genetic etiologies, respectively. In various aspects, the compositions of the disclosure may be used to treat diseases associated with a mutation of the CSF1R gene. In some aspects, the compositions of the disclosure may be used to treat leukodystrophies, such as ALSP or BANDDOS (Brain abnormalities, neurodegeneration, and dysosteosclerosis). Mutations in the following genes are implicated in diseases that are characterized by microglial dysfunction, including but not limited to: AARS1, AARS1, TREX1, RNASEH2B, RNASEH2C, RNASEH2A, ADAR, IFIH1, and USPI8. It is also contemplated that the edited and engineered cells and the methods and uses thereof provided herein can also be applied to replace, correct, or repair mutations in AARS1, AARS1, TREX1, RNASEH2B, RNASEH2C, RNASEH2A, ADAR, IFIH1, and USP18 in order to treat a patient or subject in need thereof. Without being bound by theory, ALSP is believed to be caused by a gradual loss of endogenous brain microglia that are important for the normal homeostasis of white matter tracts. In some aspects, the compositions and methods of the disclosure may be effective in replacing these missing and/or defective microglia with healthy human microglia that may prevent or slow progression of the disease.

The present disclosure further relates to methods of treating diseases in a subject in need thereof associated with a mutation of a CSF1R gene in a subject comprising administering an effective amount of the compositions comprising the iMGLs, microglial progenitor cells, myeloid cells, or myeloid cells of the disclosure. In some aspects, the disease associated with a mutation of the CSF1R gene is a leukodystrophy, such as ALSP or BANDDOS. In some aspects, the method involves administering the edited cells of the disclosure in any amount that is effective in treating or reducing the severity of a disease associated with a mutation of the CSF1R gene. In some aspects, the methods may comprise administering the compositions according to the disclosure once or several times, also intermittently, for instance on a daily basis for several days, weeks or months, and in different dosages.

In various aspects, the methods include administering a composition comprising a cell comprising a synthetic allele (e.g., synthetic gene) of or repaired human CSF1R gene to a subject suffering from a disease associated with a mutation of the CSF1R gene. In other aspects, the methods of the disclosure may include administering a composition comprising a plurality of cells characterized in that >90% or >80%, or >70% or >60% or >50% or >40% or >30% or >20% or >10% of the cells express at least one canonical microglial marker to a subject suffering from a disease associated with a mutation of the CSF1R gene. In some aspects, the methods of the disclosure may include administering a composition comprising a plurality of cells characterized in that >90% of the cells express at least one canonical microglial marker to a subject suffering from a disease associated with a mutation of the CSF1R gene.

In various aspects, the methods include administering a composition comprising an iPSC-derived microglia-lineage cell to a subject suffering from a disease associated with a mutation of the CSF1R gene. In various aspects, the iPSC-derived microglia-lineage cell is derived from autologous PSCs, genetically edited autologous PSCs, or allogeneic PSCs.

Provided herein is a method of treating or preventing a leukodystrophy in a subject, comprising: (i) obtaining a cell from the subject, wherein the cell was isolated from the subject or generated by culturing the cell isolated from the subject, (ii) producing an edited cell by repairing or replacing a defective gene in the cell from the subject; and (iii) transplanting the edited cell into the subject. In some embodiments, the obtaining can include any of the means described above for obtaining a cell from a donor (e.g., biopsy, blood sample, cerebrospinal fluid sample, bone marrow, etc.). In some embodiments, the subject was diagnosed with the leukodystrophy. In some embodiments, the subject is a risk of developing the leukodystrophy. In some embodiments, the leukodystrophy is Adult-Onset Leukoencephalopathy or BANDDOS. In some embodiments, the obtained cell is a stem cell, pluripotent stem cell, a myeloid cell, or a fibroblast. In some embodiments, the stem cell is an iPSC or an HSC. In some embodiments, the cell from the subject was derived from an iPSC. In some embodiments, the cell from the subject is an iMGL, an HPC, or a microglial precursor cell. In some embodiments, the edited cell differentiates into a microglial cell and engrafts into a microglial niche in a brain of the subject. In some embodiments, the defective gene comprises a point mutation and the repairing comprises contacting the cell with Cas9, a guide RNA comprising a sequence of the defective gene, and a single-stranded DNA oligonucleotide repair template. In some instances, the repairing of the defective gene (e.g., defective CSF1R gene) can be performed according to any of the methods described herein. In some instances, the repairing can be performed by the CRISPR-mediated repairing strategy described above. In some embodiments, the defective gene comprises a CSF1R mutation. In some embodiments, the replacing comprises inserting a CSF1R coding sequence 3′ of the FIRE of the defective CSF1R gene according to any of the methods of the disclosure for inserting a synthetic CSF1R gene into a defective CSF1R gene locus, including but not limited to the insertion of the CSF1R cDNA fragment described above. In some embodiments, the transplanting comprises injecting the edited cell into a brain or a spinal cord of the subject. The administration of the edited cell into the subject may include any route of administration described below.

### Length of Treatment and Assessment

The therapeutic efficacy of the method of treating or preventing a leukodystrophy may be assessed over a period of time since the transplantation of the edited cells described herein was performed.

In some embodiments, a period of at least about 1 week, 2 weeks, 3 weeks, 4 weeks, 5 month, 6 weeks, 7 weeks, 8 weeks, 9 weeks, 10 weeks, 11 weeks, 12 weeks, 4 months, 5 months, 6 months, 7 months, 8 months, 10 months, 1 year, 1.5 years, 2 years, 2.5 years, 3 years, 4 years, or 5 years has passed since the transplanting of the edited cells provided herein. In some embodiments, a period of at least about 2 weeks, 3 weeks, 4 weeks, 5 month, 6 weeks, 7 weeks, 8 weeks, 9 weeks, 10 weeks, 11 weeks, or 12 weeks has passed since the transplanting of the edited cells provided herein. In some embodiments, a period of at least about 4 weeks has passed since the transplanting of the edited cells provided herein. In some embodiments, a period of at least about 6 weeks has passed since the transplanting of the edited cells provided herein. In some embodiments, a period of at least about 8 weeks has passed since the transplanting of the edited cells provided herein. In some embodiments, a period of at least about 12 weeks has passed since the transplanting of the edited cells provided herein. In some embodiments, a period of no more than about 2 weeks, 3 weeks, 4 weeks, 5 month, 6 weeks, 7 weeks, 8 weeks, 9 weeks, 10 weeks, 11 weeks, 12 weeks, 4 months, 5 months, 6 months, 7 months, 8 months, 10 months, or 1 year has passed since the transplanting of the edited cells provided herein. In some embodiments, a period of about 4 weeks has passed since the transplanting of the edited cells provided herein. In some embodiments, a period of about 6 weeks has passed since the transplanting of the edited cells provided herein. In some embodiments, a period of about 8 weeks has passed since the transplanting of the edited cells provided herein. In some embodiments, a period of about 12 weeks has passed since the transplanting of the edited cells provided herein.

### Therapeutic Improvements Resultant of the Method of Treating a Leukodystrophy

The method of treating a leukodystrophy provided herein can cause an improvement of a leukodystrophy-associated phenotype, including but not limited to microbleeds, accumulation of pro-inflammatory cytokines or chemokines, calcification of blood vessels in the brain of the subject, etc. In another instance, the method of treating a leukodystrophy provided herein can delay the onset of or reduce the progression of a leukodystrophy-associated phenotype, including but not limited to microbleeds, accumulation of pro-inflammatory cytokines or chemokines, calcification of blood vessels in the brain of the subject, etc.

Provided herein is a method of transplanting the edited iMGLs, HPCs, MPCs, or MCs (generically and collectively referred to as “edited cells” henceforth) into a brain of a subject. In some embodiments, an improvement in the subject following transplantation of the edited cells comprises increasing the number or density of Iba1-expressing edited microglia in the brain of the subject after a time period as described above has passed since the transplantation of the edited iMGL, HPC, microglial precursor cell, or myeloid cell into the subject. In some embodiments, an improvement in the subject following transplantation of the edited cells comprises increasing the number, density, or average size of excitatory synapses in the brain of the subject, after a time period as described above has passed since the transplantation of the edited iMGL, HPC, microglial precursor cell, or myeloid cell into the subject. In some embodiments, an improvement in the subject following transplantation of the edited cells comprises increasing the PSD95 or NSE expression in the brain of the subject, after a time period as described above has passed since the transplantation of the edited iMGL, HPC, microglial precursor cell, or myeloid cell into the subject. In some embodiments, an improvement in the subject following transplantation of the edited cells comprises decreasing the accumulation of secreted osteopontin (OPN) in the brain of the subject, after a time period as described above has passed since the transplantation of the edited iMGL, HPC, microglial precursor cell, or myeloid cell into the subject. In some embodiments, an improvement in the subject following transplantation of the edited cells comprises decreasing the frequency of axonal spheroids immunoreactive for neurofilament SMI312 and LAMP1 in the brain of the subject, after a time period as described above has passed since the transplantation of the edited iMGL, HPC, microglial precursor cell, or myeloid cell into the subject. In some embodiments, an improvement in the subject following transplantation of the edited cells comprises decreasing the number, density, or frequency of hydroxyapatite calcium crystals in the brain of the subject, after a time period as described above has passed since the transplantation of the edited iMGL, HPC, microglial precursor cell, or myeloid cell into the subject. In some embodiments, an improvement in the subject following transplantation of the edited cells comprises decreasing the levels of Tau phosphorylated at Thr217 (pTau217) in the brain, blood plasma, or cerebrospinal fluid of the subject, after a time period as described above has passed since the transplantation of the edited iMGL. HPC, microglial precursor cell, or myeloid cell into the subject. In some embodiments, an improvement in the subject following transplantation of the edited cells comprises decreasing the GFAP expression levels in the brain, cerebrospinal fluid, or blood plasma of the subject, after a time period as described above has passed since the transplantation of the edited iMGL, HPC, microglial precursor cell, or myeloid cell into the subject. In some embodiments, an improvement in the subject following transplantation of the edited cells comprises decreasing the MCP-1 expression levels in the brain of the subject, after a time period as described above has passed since the transplantation of the edited iMGL, HPC, microglial precursor cell, or myeloid cell into the subject.

In some cases, an improvement in the subject following transplantation of the edited cells comprises decreasing the expression levels in the brain, cerebrospinal fluid, or blood plasma of the subject the expression levels of at least one of SERPINA3N, neurofilament light chain (NFL), GFAP, pTau217, MCP-1 (CCL2), Osteopontin (SPP1), LAMP1, or LAMP2 genes. In some embodiments, the improvement in the subject following transplantation of the edited cells can include expression levels of SERPINA3N, neurofilament light chain (NFL), GFAP, pTau217, MCP-1 (CCL2), Osteopontin (SPP1), LAMP1, or LAMP2 genes that are decreased by at least 20% after a time period as described above has passed since the transplantation of the edited iMGL, HPC, microglial precursor cell, or myeloid cell into the subject. In some embodiments, the improvement in the subject following transplantation of the edited cells can include expression levels of SERPINA3N, neurofilament light chain (NFL), GFAP, pTau217, MCP-1 (CCL2), Osteopontin (SPP1), LAMP1, or LAMP2 genes that are decreased by at least 20%, 30%, 40%, 50%, 60%, 70%, or 80% after a time period as described above has passed since the transplantation of the edited iMGL, HPC, microglial precursor cell, or myeloid cell into the subject.

In some embodiments, an improvement in the subject following transplantation of the edited cells comprises any two of: increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SMI312 and LAMP1 in the brain of the subject; decreasing the number, density, or frequency of hydroxyapatite calcium crystals in the brain of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; or decreasing the MCP-1 expression levels in the brain of the subject, as described above has passed since the transplantation of the edited iMGL, HPC, microglial precursor cell, or myeloid cell into the subject after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

In some embodiments, an improvement in the subject following transplantation of the edited cells comprises any three of: increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SMI312 and LAMP1 in the brain of the subject; decreasing the number, density, or frequency of hydroxyapatite calcium crystals in the brain or blood plasma of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; or decreasing the MCP-1 expression levels in the brain of the subject, as described above has passed since the transplantation of the edited iMGL, HPC, microglial precursor cell, or myeloid cell into the subject after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

In some embodiments, the improvement in the subject following transplantation of the edited cells comprises increasing the number, density, or average size of excitatory synapses in the brain of the subject; increasing the PSD95 or NSE expression in the brain of the subject; decreasing the accumulation of secreted osteopontin (OPN) in the brain of the subject; decreasing the frequency of axonal spheroids immunoreactive for neurofilament SMI312 and LAMP1 in the brain of the subject; decreasing the number, density, or frequency of hydroxyapatite calcium crystals in the brain of the subject; decreasing the levels of Tau phosphorylated at Thr217 (pTau217) in the brain, cerebrospinal fluid, or blood plasma of the subject; decreasing the GFAP expression levels in the brain, cerebrospinal fluid, or blood plasma of the subject; and decreasing the MCP-1 expression levels in the brain of the subject, after a time period as described above has passed since the transplantation of the edited iMGL, HPC, microglial precursor cell, or myeloid cell into the subject. Preferably, the period of time following the transplanting is at least about 4 weeks. Also preferably, the period of time following the transplanting is at least about 6 weeks.

### Subject In Need Thereof

In various instances, the compositions and methods of edited cells described herein are administered and transplanted into a subject in need thereof. In some embodiments, the subject was diagnosed with a leukodystrophy. In some embodiments, the subject was diagnosed with ALSP. In some embodiments, the subject was diagnosed with BANDDOS. In some embodiments, the subject was diagnosed with a CSF1R-related disorder. In some embodiments, the subject was diagnosed with a condition comprising a microglial depletion, especially in the brain of the subject. In some embodiments, the subject is at-risk of developing a leukodystrophy. In some embodiments, the subject is at-risk of developing ALSP. In some embodiments, subject is at-risk of developing BANDDOS. In some embodiments, the subject is at-risk of developing a CSF1R-related disorder. In some embodiments, the subject is at-risk of developing a condition comprising a microglial depletion. In some embodiments, the subject has a CSF1R haploinsufficiency. In some embodiments, the subject is homozygous for a defective CSF1R gene. In some embodiments, the subject is heterozygous for a defective CSF1R gene.

In some embodiments, the subject comprises a mammal. In some embodiments, the subject comprises a mouse, rat, dog, cat, pig, rabbit, guinea pig, horse, or camel. In some embodiments, the subject comprises a primate. In some embodiments, the subject comprises a non-human primate. In some embodiments, the subject comprises a human. In some embodiments, the human is an adult human. In some embodiments, the human is a child. In some embodiments, the human is an adolescent human. In some embodiments, the human is a prepubescent human. In some embodiments, the human presents symptoms of a leukodystrophy. In some embodiments, the human presents symptoms of ALSP. In some embodiments, the human presents symptoms of BANDDOS. In some embodiments, the symptoms of ALPS comprise microbleeds in the brain, brain calcification, formation of axonal spheroids, accumulation of inflammatory cytokines or chemokines, or a decreased microglia population in the brain, neuronal cell death, neurodegeneration, synaptic atrophy, or loss, astrogliosis, formation of neurofilaments in the brain, or any other symptom described above.

One aspect of the disclosure provides a therapeutic composition comprising: a pharmaceutical carrier; and an engineered iPSC-derived microglia or iPSC-derived HPC comprising a Colony Stimulating Factor 1 Receptor (CSF1R) gene, wherein the CSF1R gene comprises a CSF1R coding sequence (CDS) inserted 3′ of a fms-intronic regulatory element (FIRE) and 5′ of a mutation in the CSF1R gene, wherein the CDS comprises a polyadenylation signal to prevent transcription of the mutation in the CSF1R gene, wherein the CDS encodes wildtype CSF1R (e.g., human CSF1R). In some embodiments, the CDS comprises a silent mutation effective for preventing enzyme-mediated DNA cleavage. The aforementioned cell types may be human cells.

One aspect of the disclosure provides a therapeutic composition comprising: a pharmaceutical carrier; and an engineered iPSC-derived microglial cell or an iPSC-derived HPC, comprising a Colony Stimulating Factor 1 Receptor (CSF1R) gene, wherein the CSF1R gene comprises a CSF1R coding sequence (CDS) inserted in Exon 2, wherein the CDS encodes wild type CSF1R and comprises a polyadenylation signal to prevent transcription of Exons 3-22 of the CSF1R gene. In some embodiments, the CDS comprises a silent mutation effective for preventing enzyme-mediated DNA cleavage. The aforementioned cell types may be human cells.

One aspect of the disclosure provides a therapeutic composition, e.g., injectable composition, comprising: a pharmaceutical carrier; and a population of engineered iPSC-derived microglia or iPSC-derived HPCs comprising a Colony Stimulating Factor 1 Receptor (CSF1R) gene, wherein the CSF1R gene comprises a CSF1R coding sequence (CDS) inserted 3′ of a fms-intronic regulatory element (FIRE) and 5′ of a mutation in the CSF1R gene, wherein the CDS comprises a polyadenylation signal to prevent transcription of the mutation in the CSF1R gene, wherein the CDS encodes wildtype CSF1R (e.g., human CSF1R). In some embodiments, at least 80% of cells in the population of engineered iPSC-derived microglia or iPSC-derived HPCs express wildtype CSF1R. In some embodiments, at least 90% of the cells in the population of iPSC-derived microglia iPSC-derived HPCs express wildtype CSF1R. In some embodiments, at least 95% of the cells in the population of iPSC-derived microglia iPSC-derived HPCs express wildtype CSF1R. In some embodiments, at least 99% of the cells in the population of iPSC-derived microglia iPSC-derived HPCs express wildtype CSF1R. In some embodiments, the CDS comprises a silent mutation effective for preventing enzyme-mediated DNA cleavage. The aforementioned cell types may be human cells.

One aspect of the disclosure provides a therapeutic composition, e.g., injectable composition, comprising: a pharmaceutical carrier; and a population of engineered iPSC-derived microglia or iPSC-derived HPCs comprising a Colony Stimulating Factor 1 Receptor (CSF1R) gene, wherein the CSF1R gene comprises a CSF1R coding sequence (CDS) inserted in Exon 2, wherein the CDS comprises a polyadenylation signal to prevent transcription of Exons 3-22 of the CSF1R gene, wherein the CDS encodes wildtype CSF1R (e.g., human CSF1R). In some embodiments, at least 80% of cells in the population of engineered iPSC-derived microglia or iPSC-derived HPCs express wildtype CSF1R. In some embodiments, at least 90% of the cells in the population of iPSC-derived microglia express iPSC-derived HPCs wildtype CSF1R. In some embodiments, at least 95% of the cells in the population of iPSC-derived microglia iPSC-derived HPCs express wildtype CSF1R. In some embodiments, at least 99% of the cells in the population of iPSC-derived microglia iPSC-derived HPCs express wildtype CSF1R. In some embodiments, the CDS comprises a silent mutation effective for preventing enzyme-mediated DNA cleavage. The aforementioned cell types may be human cells.

Referring to the disclosures herein, including the embodiments above, the mutation in the CSF1R gene may be one associated with a leukodystrophy, e.g., Adult-Onset Leukodystrophy (ALSP).

As previously discussed, the CDS may comprise a silent mutation effective for preventing enzyme-mediated DNA cleavage. An example is shown in SEQ ID NO: 2 (see Table 8), wherein a silent mutation is introduced at nucleotide 5 to prevent cleavage by Cas9.

One aspect of the disclosure provides a guide RNA according to SEQ ID NO: 5.

One aspect of the disclosure provides a guide RNA according to SEQ ID NO: 12.

One aspect of the disclosure provides a guide RNA according to SEQ ID NO: 13.

One aspect of the disclosure provides a guide RNA according to SEQ ID NO: 14.

One aspect of the disclosure provides a guide RNA according to SEQ ID NO: 15.

One aspect of the disclosure provides a guide RNA according to SEQ ID NO: 16.

One aspect of the disclosure provides a single-stranded oligodeoxynucleotide according to SEQ ID NO: 7.

One aspect of the disclosure provides a single-stranded oligodeoxynucleotide according to SEQ ID NO: 8.

One aspect of the disclosure provides a single-stranded oligodeoxynucleotide according to SEQ ID NO: 9.

One aspect of the disclosure provides a single-stranded oligodeoxynucleotide according to SEQ ID NO: 10.

One aspect of the disclosure provides a single-stranded oligodeoxynucleotide according to SEQ ID NO: 11.

One aspect of the disclosure provides the use of a composition comprising an engineered cell (e.g., human cell) for a method of treating or preventing a leukodystrophy in a subject in need thereof, the method comprising a step of administering the composition to the subject wherein the composition is effective for replacing a portion of microglia in a brain of the subject, characterized in that the composition comprises an engineered cell according to the disclosure herein. The composition may be effective for replacing a portion of microglia in the brain of a subject. The composition may be effective for achieving a therapeutic concentration of microglia expressing non-mutated CSF1R in a brain of the subject. As an example, the engineered cell may comprise a synthetic repaired or replaced Colony Stimulating Factor 1 Receptor (CSF1R) gene, e.g., human CSF1R gene. In some embodiments, the synthetic repaired or replaced CSF1R gene comprises an inserted CSF1R coding sequence or fragment thereof located 3′ of the fms-intronic response element (FIRE). In some embodiments, the synthetic repaired or replaced CSF1R gene comprises a silent mutation. In some embodiments, the synthetic CSF1R gene further comprises a stop codon and a poly-A signal. In some embodiments, the synthetic CSF1R gene comprises the nucleic acid sequence of SEQ ID NO: 2. As another example, the engineered cell may comprise a Colony Stimulating Factor 1 Receptor (CSF1R) gene, wherein the CSF1R gene comprises a CSF1R coding sequence (CDS) encoding wild type CSF1R positioned 3′ of a fms-intronic regulatory element (FIRE) and 5′ of a mutation in the CSF1R gene, the CDS comprises a polyadenylation signal to prevent transcription of the mutation. In some embodiments, the CDS comprises a silent mutation effective for preventing enzyme-mediated DNA cleavage, e.g., Cas9 cleavage. In some embodiments, the engineered cell is a microglia-like cell (MGL). In some embodiments, the engineered cell is a microglial progenitor cell or a hematopoietic stem cell (HSC).

In some embodiments, the engineered cell is an induced pluripotent stem cell (iPSC). In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

One aspect of the disclosure provides the use of a composition comprising an engineered cell (e.g., human cell) in the manufacture of a medicament for treating or preventing a leukodystrophy in a subject in need thereof, the composition comprising an engineered cell according to the disclosure herein. In some embodiments, the medicament comprises a pharmaceutical carrier. In some embodiments, the medicament is injectable. As an example, the engineered cell may comprise a synthetic repaired or replaced Colony Stimulating Factor 1 Receptor (CSF1R) gene, e.g., human CSF1R gene. In some embodiments, the synthetic repaired or replaced CSF1R gene comprises an inserted CSF1R coding sequence or fragment thereof located 3′ of the fms-intronic response element (FIRE). In some embodiments, the synthetic repaired or replaced CSF1R gene comprises a silent mutation. In some embodiments, the synthetic CSF1R gene further comprises a stop codon and a poly-A signal. In some embodiments, the synthetic CSF1R gene comprises the nucleic acid sequence of SEQ ID NO: 2. As another example, the engineered cell may comprise a Colony Stimulating Factor 1 Receptor (CSF1R) gene, wherein the CSF1R gene comprises a CSF1R coding sequence (CDS) encoding wild type CSF1R positioned 3′ of a fms-intronic regulatory element (FIRE) and 5′ of a mutation in the CSF1R gene, the CDS comprises a polyadenylation signal to prevent transcription of the mutation. In some embodiments, the CDS comprises a silent mutation effective for preventing enzyme-mediated DNA cleavage, e.g., Cas9 cleavage. In some embodiments, the engineered cell is a microglia-like cell (MGL). In some embodiments, the engineered cell is an induced pluripotent stem cell (iPSC)-derived microglia cell. In some embodiments, the engineered cell is a microglial progenitor cell or a hematopoietic stem cell (HSC). In some embodiments, the engineered cell is an induced pluripotent stem cell (iPSC). In some embodiments, the engineered cell is a microglial progenitor, a hematopoietic stem cell (HSC), or induced pluripotent stem cell. In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

One aspect of the disclosure provides the use of a composition comprising an engineered cell (e.g., human cell) for a method of treating or preventing a leukodystrophy in a subject in need thereof, the method comprising a step of administering the composition to the subject, wherein the composition is effective for achieving a therapeutic concentration of microglia expressing non-mutated CSF1R in a brain of the subject, characterized in that the composition comprises an engineered cell comprising a Colony Stimulating Factor 1 Receptor (CSF1R) gene, the CSF1R gene comprises a CSF1R coding sequence (CDS) encoding wild type CSF1R positioned 3′ of a fms-intronic regulatory element (FIRE) and 5′ of a mutation in the CSF1R gene, the CDS comprises a polyadenylation signal to prevent transcription of the mutation. In some embodiments, the CDS comprises a silent mutation effective for preventing enzyme-mediated DNA cleavage by Cas9. In some embodiments, the engineered cell is a microglia-like cell (MGL). In some embodiments, the engineered cell is an induced pluripotent stem cell (iPSC)-derived microglia cell. In some embodiments, the engineered cell is a microglial progenitor cell or a hematopoietic stem cell (HSC). In some embodiments, the engineered cell is an induced pluripotent stem cell (iPSC). In some embodiments, the engineered cell is a microglial progenitor, a hematopoietic stem cell (HSC), or induced pluripotent stem cell. In some embodiments, the engineered cell is a myeloid cell, a hematopoietic precursor cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, or a yolk-sac-derived myeloid cell.

### Administration of Engineered Cells

Any of the methods provided herein may involve administering the MCs, MPCs, HPCs, MLCs, or macrophages to a subject.

Unlike native microglia cells or macrophages, the edited cells (e.g., MCs, macrophages, etc.) of this disclosure, may, in some instances, relocate and infiltrate into the CNS by passing through a blood-brain barrier (BBB) of a host subject (e.g., a human subject).

Edited cells generated according to the methods provided herein may be administered orally, though compositions of similar type are most frequently delivered parenterally, particularly intravenously, intramuscularly, transdermally, intradermally, intratracheally, intranasally, stereotactically, or subretinally. In an animal body, it can prove advantageous to apply the pharmaceutical compositions as described above via an intravenous injection or by direct injection into the target tissues. For systemic application, the intravenous, intravascular, intramuscular, intranasal, stereotactic, intraparenchymal, intracerebroventricular, subretinal or intrathecal routes are preferred. A more local application may be effected subcutaneously, intradermally, intracutaneously, intralobally, intramedullarly, or directly in or near the tissue to be treated. Depending on the desired duration and effectiveness of the treatment, the compositions according to the disclosure may be administered once or several times, also intermittently, for instance on a daily basis for several days, weeks or months, and in different dosages. In some embodiments, the edited and differentiated iMGL, HPC or microglial precursor cell is administered into the subject via a stereotactic, intraparenchymal, intracerebroventricular, intrathecal, subretinal, intraperitoneal, or intranasal injection. In some embodiments, the edited and differentiated iMGL, HPC or microglial precursor cell is administered into the subject via an intraperitoneal or intravenous injection.

In various aspects, the compositions of the disclosure may be administered in any amount that is effective in the treatment of a disease associated with a mutation of a CSF1R gene. It is understood that the compositions described herein may be administered to any animal susceptible to diseases associated with a mutation of the CSF1R gene including humans and other mammals. Further, administration of any of the compositions described herein may be effective in reducing the severity of diseases associated with a mutation of the CSF1R gene.

### Pharmaceutically Acceptable Formulation

In some aspects, the cells that express a canonical microglial marker may be formulated with a pharmaceutically acceptable carrier to produce a composition that may be administered to treat diseases associated with a mutation of the CSF1R gene. In various aspects, the compositions may include a plurality of cells characterized in that >90% or >80%, or >70% or >60% or >50% or >40% or >30% or >20% or >10% of the cells express a canonical microglial marker. In some aspects, the compositions may include a plurality of cells characterized in that >90% of the cells express a canonical microglial marker.

In various aspects, iMGLs or microglial progenitor cells of the disclosure may be generated from autologous PSCs from a subject and transplanted into the subject to treat a disease associated with a mutation of a microglial gene. In some aspects, iMGLs or microglial progenitor cells of the disclosure may be generated from autologous PSCs from a subject, and transplanted into the subject to supplement microglia numbers within the central nervous system of the subject.

In various aspects, the cells and/or compositions of the disclosure may be transported in a frozen form or under tight temperature regulation at just above freezing. The cells may be frozen using methods known in the art, such as cryopreservation. In various aspects, the cells may be transported to a medical facility in a frozen or substantially frozen form, thawed, and then directly administered to a subject using the routes of administration described herein. In other aspects, the cells may be cultured for several days to allow the cells to recover homeostasis after phagocytic clearance of any non-viable cells.

In various aspects, the compositions of the disclosure may include an edited cell as described above comprising a synthetic allele of a human CSF1R gene formulated with a pharmaceutically acceptable carrier. In other aspects, the compositions of the disclosure may include a plurality of cells characterized in that >90% or >80%, or >70% or >60% or >50% or >40% or >30% or >20% or >10% of the cells express a canonical microglial marker, formulated with a pharmaceutically acceptable carrier. In some aspects, the compositions of the disclosure may include a plurality of cells characterized in that >90% of the cells express a canonical microglial marker formulated with a pharmaceutically acceptable carrier. Alternatively, or additionally, compositions of this disclosure may comprise a plurality of cells characterized in that >90% or >80%, or >70% or >60% or >50% or >40% or >30% or >20% or >10% of said cells comprise at least one synthetic allele of (i.e., are heterozygous, homozygous or hemizygous for) a gene of interest such as the CSF1R gene. In some cases, >90% or >80%, or >70% or >60% or >50% or >40% or >30% or >20% or >10% of said cells are both (a) heterozygous, homozygous, or hemizygous for the at least one synthetic allele of the gene of interest, and (b) express one or more canonical microglial markers.

In various aspects, the compositions of the disclosure may include one or more pharmaceutically acceptable carriers. As used herein, “a pharmaceutically-acceptable carrier” includes any and all solvents, dispersion media, coatings, stabilizing agents, diluents, preservatives, antibacterial and antifungal agents, isotonic agents, adsorption delaying agents, and any combination thereof. In some aspects, the pharmaceutically acceptable carrier is selected from the group consisting of a solvent, a dispersion media, a coating, a stabilizing agent, a preservative, an antimicrobial agent, an antifungal agent, an isotonic agent, an adsorption delaying agent, and any combination thereof.

In various aspects, the compositions described herein may be formulated as an injectable formulation. In some aspects, the compositions described herein may be administered by the systemic route, in particular by a parenteral route. In some aspects, the compositions may be administered by an intravenous, intramuscular, intradermal, intraparenchymal, intracerebroventricular, intrathecal, intranasal, stereotactic, subretinal or subcutaneous route, or by an oral route.

The administration modes, dosages and optimum pharmaceutical formulations may be determined according to criteria generally taken into account in the establishment of a treatment adapted to an animal such as, for example, the age or the weight, the seriousness of its general condition, the tolerance to the treatment and the secondary effects noted.

Parenteral formulations, designed for injection into body fluid systems, require proper isotonicity and pH buffering to the corresponding levels of body fluids. Isotonicity can be appropriately adjusted with sodium chloride and other salts as needed. Suitable solvents may be used to increase the solubility of the ingredients in the formulation and the stability of the liquid preparation. Further additives that may be employed in the present compositions include, but are not limited to, dextrose, conventional antioxidants, and conventional chelating agents. Parenteral dosage forms must also be sterilized prior to use.

It should be appreciated that all scientific and technological terms used herein have the same meaning as commonly understood by those of ordinary skill in the art.

### Kits

Provided herein is a kit related to the culture media supporting differentiation of HPCs into an iMGL or MPC. In some embodiments, the culture media comprises CSF-1, IL-34, and TGFβ1. In some embodiments, the culture media comprises one or more of CSF-1, IL-34, and TGFβ1. In some embodiments, the culture media comprises CSF-1, IL-34, and TGFβ2. In some embodiments, the culture media comprises CSF-1, IL-34, and TGFβ mimetic.

Provided herein is a kit related to the administration of any of the engineered cells provided herein (e.g., iMGLs, MPCs, HPCs, MCs, macrophages, etc.), wherein the kit comprises: a formulation of the engineered cell of this disclosure suitable for administration of the engineered cell into a subject. Described herein, in some embodiments, is a kit comprising: a composition of an engineered cell provided herein for the treatment of a leukodystrophy or microglia-related disorder. In some embodiments, the leukodystrophy is Adult-onset leukoencephalopathy (ALSP). In some embodiments, the microglia-related disorder comprises a leukodystrophy or any disease characterized by a depletion of microglia or loss of CSF1R expression in a subject in need thereof. In some embodiments, the kit further comprises: a storage container for a population of the engineered cell, wherein the storage container is selected from the group consisting of: a vial, a syringe, a capsule, a cartridge, and an ampule.

### Cell Therapy for Use in the Treatment of a Leukodystrophy

Provided herein is a cell therapy for use in the treatment of a disorder or condition associated with a leukodystrophy comprising: editing the genome of an isolated cell to repair or replace a target gene; and incubating the isolated cell in a culture media comprising a differentiation factor, thereby generating an edited and differentiated cell. In some embodiments, the target gene comprises a disease-associated mutation. In some embodiments, the disease-associated mutation is a mutation associated with a leukodystrophy. In some embodiments, the leukodystrophy comprises an Adult-onset leukoencephalopathy (ALSP). In some embodiments, the isolated cell is an isolated human cell. In some embodiments, the isolated cell is an induced pluripotent stem cell. In some embodiments, the isolated cell was derived from a stem cell. In some embodiments, the isolated cell was derived from an iPSC. In some embodiments, the isolated cell was derived from a hematopoietic stem cell (HSC), a hematopoietic progenitor cell (HPC), or a myeloid cell. In some embodiments, the edited and differentiated cell is a myeloid cell, a myeloid precursor cell, a myeloid progenitor cell, an erythro-myeloid precursor cell, an erythro-myeloid progenitor cell, a macrophage, a monocyte, a fetal macrophage, a hematopoietic stem cell-derived myeloid cell, or a yolk-sac-derived myeloid cell. In some embodiments, the edited and differentiated cell is an HPC or an MPC. In some embodiments, the edited and differentiated cell is an iMGL. In some embodiments, the editing occurs before the incubating. In some embodiments, the editing occurs after the incubating. In some embodiments, the incubating comprises a first incubation period for differentiating an iPSC into an HPC and a second incubation period for differentiating the HPC. In some embodiments, the second incubation period is 1-28 days. In some embodiments, the cell culture media for the second incubation period comprises: IL-34, CSF-1, and TGFβ1; or IL-34, CSF-1, and a TGFβ mimetic. In some embodiments, the edited and differentiated cell can differentiate further in vivo. In some embodiments, the isolated cell was derived from a sample collected from a donor. In some embodiments, the sample comprises fibroblasts. In some embodiments, the sample comprises bone marrow. In some embodiments, the sample comprises blood or cerebrospinal fluid. In some embodiments, the cell therapy for use further comprises generating an iPSC from a cell of the sample before the editing or the incubating. In some embodiments, the editing comprises repairing a disease-associated mutation. In some embodiments, the disease-associated mutation causes reduced expression of the target gene, and wherein the edited and differentiated cell expresses at least 80% as much of the target gene as an otherwise identical control cell without the disease associated mutation or the editing. In some embodiments, the disease-associated mutation causes reduced activity of a polypeptide encoded by the target gene and wherein the edited and differentiated cell has at least 80% of the activity compared to an otherwise identical control cell without the disease associated mutation or the editing. In some embodiments, the editing comprises inserting a cDNA or fragment thereof within the target gene. In some embodiments, the disease-associated mutation comprises a mutation of a CSF1R gene. In some embodiments, the CSF1R gene is a human CSF1R gene. In some embodiments, the CSF1R mutation is within a nucleotide sequence encoding a kinase domain of a CSF1R polypeptide. In some embodiments, the mutant CSF1R gene encodes a CSF1R polypeptide comprises a point mutation with respect to a CSF1R polypeptide comprising SEQ ID NO: 1 or a fragment thereof. In some embodiments, the CSF1R mutation comprises a deletion mutation or an insertion mutation. In some embodiments, the editing comprises contacting the target gene with a TALEN, a zinc-finger endonuclease, a Base editor, a Prime editor, or a meganuclease. In some embodiments, the editing comprises contacting the target gene with a CRISPR endonuclease. In some embodiments, the CRISPR endonuclease comprises a Cas9 endonuclease. In some embodiments, the editing comprises introducing into the cell: a Cas9 polypeptide or a polynucleotide encoding the Cas9 polypeptide, a gRNA comprising a sequence of the CSF1R gene or a polynucleotide encoding the gRNA, and a polynucleotide comprising a CSF1R cDNA fragment, thereby replacing the CSF1R gene. In some embodiments, the gRNA comprises SEQ ID NO: 5. In some embodiments, the CSF1R cDNA fragment comprises exons 2-21 of the human CSF1R gene. In some embodiments, the polynucleotide comprising a CSF1R cDNA fragment further comprises a stop codon and a poly-A signal. In some embodiments, the CSF1R cDNA fragment comprises a silent mutation. In some embodiments, the CSF1R cDNA encodes a polypeptide having at least 90% sequence identity to a nucleotide sequence of SEQ ID NO: 1 or the fragment thereof. In some embodiments, the edited and differentiated cell comprises CSF1R proteins having at least 80% of the CSF1R tyrosine kinase activity of an otherwise identical control cell having exactly two copies of a wildtype CSF1R gene. In some embodiments, the cell therapy for use further comprises comprising introducing into the cell: a Cas9 polypeptide or a polynucleotide encoding the Cas9 polypeptide, a gRNA complementary to a nucleotide sequence adjacent to the nucleotide sequence encoding the point mutation, and a homology-directed repair template polynucleotide comprising a wildtype CSF1R sequence at a position in the CSF1R gene corresponding to the position of the point mutation, thereby repairing the point mutation. In some embodiments, the homology-directed repair template polynucleotide is a single-stranded DNA oligonucleotide (ssODN). In some embodiments, wherein the point mutation comprises a M8751 mutation. In some embodiments, the gRNA comprises SEQ ID NO: 12. In some embodiments, the homology-directed repair template polynucleotide comprises SEQ ID NO: 7. In some embodiments, the point mutation comprises a L786S mutation. In some embodiments, the gRNA comprises SEQ ID NO: 13. In some embodiments, the homology-directed repair template polynucleotide comprises SEQ ID NO: 8. In some embodiments, the point mutation comprises a M785T mutation. In some embodiments, the gRNA comprises SEQ ID NO: 14. In some embodiments, the homology-directed repair template polynucleotide comprises SEQ ID NO: 9. In some embodiments, the point mutation comprises a N854K mutation. In some embodiments, the gRNA comprises SEQ ID NO: 15. In some embodiments, the homology-directed repair template polynucleotide comprises SEQ ID NO: 10. In some embodiments, the point mutation comprises a G598E mutation. In some embodiments, the gRNA comprises SEQ ID NO: 16. In some embodiments, the homology-directed repair template polynucleotide comprises SEQ ID NO: 11. In some embodiments, a proliferation rate of the edited and differentiated cell, is greater than a proliferation rate of an otherwise identical control iMGL, HPC or microglial precursor cell without the editing. In some embodiments, a transcriptional profile of microglia-related genes of the edited and differentiated iMGL more closely resembles a transcriptional profile of the microglia-related genes in a positive control iMGL or microglial precursor cell with exactly two native, wildtype CSF1R alleles compared to a transcriptomic profile of the microglia-related genes in an otherwise identical negative control iMGL comprising a mutant CSF1R gene. In some embodiments, the cell therapy for use further comprises transplanting the edited and differentiated iMGL, HPC or MPC into a brain of a subject. In some embodiments, the transplanting: increases number or density of Iba1-expressing edited and differentiated microglia in the brain; increases number, density, or average size of excitatory synapses in the brain; increases PSD95 or NSE expression in the brain; decreases accumulation of secreted osteopontin (OPN) in the brain; decreases frequency of axonal spheroids immunoreactive for neurofilament SMI312 and LAMP1 in the brain; decreases number, density, or frequency of hydroxyapatite calcium crystals; decreases levels of Tau phosphorylated at Thr217 (pTau217) in the brain; decreases GFAP expression levels in the brain; or decreases MCP-1 expression levels in the brain, wherein at least 6 weeks has passed since the transplantation of the edited and differentiated iMGL, HPC, microglial precursor cell, or myeloid cell into the subject. In some embodiments, the transplanting is autologous and the donor is the subject. In some embodiments, the transplanting is allogeneic and the donor is not the subject. In some embodiments, the edited and differentiated iMGL, HPC, or microglial precursor cell engrafts into and repopulates the brain of a subject with a leukodystrophy. In some embodiments, the edited and differentiated iMGL. HPC or microglial precursor cell is administered into the subject via a stereotactic, intraparenchymal, intracerebroventricular, intrathecal, subretinal, intraperitoneal, or intranasal injection. In some embodiments, the edited and differentiated iMGL, HPC or microglial precursor cell is administered into the subject via an intraperitoneal or intravenous injection.

### Use of an Edited and Differentiated Cell Population for the Manufacturing of a Medicament

Provided herein is a method for use of an edited and differentiated cell in the manufacturing of a medicament for the treatment of a leukodystrophy, the method comprising: editing the genome of an isolated cell to repair or replace a target gene; and incubating the isolated cell in a culture media comprising a differentiation factor, thereby generating an edited and differentiated cell. In some embodiments, the target gene comprises a disease-associated mutation. In some embodiments, the disease-associated mutation is a mutation associated with a leukodystrophy. In some embodiments, the leukodystrophy comprises an Adult-onset leukoencephalopathy (ALSP). In some embodiments, the isolated cell is an isolated human cell. In some embodiments, the isolated cell is an induced pluripotent stem cell. In some embodiments, the isolated cell was derived from a stem cell. In some embodiments, the isolated cell was derived from an iPSC. In some embodiments, the isolated cell was derived from a hematopoietic stem cell (HSC), a hematopoietic progenitor cell (HPC), or a myeloid cell. In some embodiments, the edited and differentiated cell is a myeloid cell, a myeloid precursor cell, a myeloid progenitor cell, an erythro-myeloid precursor cell, an erythro-myeloid progenitor cell, a macrophage, a monocyte, a fetal macrophage, a hematopoietic stem cell-derived myeloid cell, or a yolk-sac-derived myeloid cell. In some embodiments, the edited and differentiated cell is an HPC or an MPC. In some embodiments, the edited and differentiated cell is an iMGL. In some embodiments, the editing occurs before the incubating. In some embodiments, the editing occurs after the incubating. In some embodiments, the incubating comprises a first incubation period for differentiating an iPSC into an HPC and a second incubation period for differentiating the HPC.

In some embodiments, the second incubation period is 1-28 days. In some embodiments, the cell culture media for the second incubation period comprises: IL-34, CSF-1, and TGFβ1; or IL-34, CSF-1, and a TGFβ mimetic. In some embodiments, the edited and differentiated cell can differentiate further in vivo. In some embodiments, the isolated cell was derived from a sample collected from a donor. In some embodiments, the sample comprises fibroblasts. In some embodiments, the sample comprises bone marrow. In some embodiments, the sample comprises blood or cerebrospinal fluid. In some embodiments, the method for use further comprises generating an iPSC from a cell of the sample before the editing or the incubating. In some embodiments, the editing comprises repairing a disease-associated mutation. In some embodiments, the disease-associated mutation causes reduced expression of the target gene, and wherein the edited and differentiated cell expresses at least 80% as much of the target gene as an otherwise identical control cell without the disease associated mutation or the editing. In some embodiments, the disease-associated mutation causes reduced activity of a polypeptide encoded by the target gene and wherein the edited and differentiated cell has at least 80% of the activity compared to an otherwise identical control cell without the disease associated mutation or the editing. In some embodiments, the editing comprises inserting a cDNA or fragment thereof within the target gene. In some embodiments, the disease-associated mutation comprises a mutation of a CSF1R gene. In some embodiments, the CSF1R gene is a human CSF1R gene. In some embodiments, the CSF1R mutation is within a nucleotide sequence encoding a kinase domain of a CSF1R polypeptide. In some embodiments, the mutant CSF1R gene encodes a CSF1R polypeptide comprises a point mutation with respect to a CSF1R polypeptide comprising SEQ ID NO: 1 or a fragment thereof. In some embodiments, the CSF1R mutation comprises a deletion mutation or an insertion mutation. In some embodiments, the editing comprises contacting the target gene with a TALEN, a zinc-finger endonuclease, a Base editor, a Prime editor, or a meganuclease. In some embodiments, the editing comprises contacting the target gene with a CRISPR endonuclease. In some embodiments, the CRISPR endonuclease comprises a Cas9 endonuclease. In some embodiments, the editing comprises introducing into the cell: a Cas9 polypeptide or a polynucleotide encoding the Cas9 polypeptide, a gRNA comprising a sequence of the CSF1R gene or a polynucleotide encoding the gRNA, and a polynucleotide comprising a CSF1R cDNA fragment, thereby replacing the CSF1R gene. In some embodiments, the gRNA comprises SEQ ID NO: 5. In some embodiments, the CSF1R cDNA fragment comprises exons 2-21 of the human CSF1R gene. In some embodiments, the polynucleotide comprising a CSF1R cDNA fragment further comprises a stop codon and a poly-A signal. In some embodiments, the CSF1R cDNA fragment comprises a silent mutation. In some embodiments, the CSF1R cDNA encodes a polypeptide having at least 90% sequence identity to a nucleotide sequence of SEQ ID NO: 1 or the fragment thereof. In some embodiments, the edited and differentiated cell comprises CSF1R proteins having at least 80% of the CSF1R tyrosine kinase activity of an otherwise identical control cell having exactly two copies of a wildtype CSF1R gene. In some embodiments, the method for use further comprises comprising introducing into the cell: a Cas9 polypeptide or a polynucleotide encoding the Cas9 polypeptide, a gRNA complementary to a nucleotide sequence adjacent to the nucleotide sequence encoding the point mutation, and a homology-directed repair template polynucleotide comprising a wildtype CSF1R sequence at a position in the CSF1R gene corresponding to the position of the point mutation, thereby repairing the point mutation. In some embodiments, the homology-directed repair template polynucleotide is a single-stranded DNA oligonucleotide (ssODN). In some embodiments, wherein the point mutation comprises a M875I mutation. In some embodiments, the gRNA comprises SEQ ID NO: 12. In some embodiments, the homology-directed repair template polynucleotide comprises SEQ ID NO: 7. In some embodiments, the point mutation comprises a L786S mutation. In some embodiments, the gRNA comprises SEQ ID NO: 13. In some embodiments, the homology-directed repair template polynucleotide comprises SEQ ID NO: 8. In some embodiments, the point mutation comprises a M785T mutation. In some embodiments, the gRNA comprises SEQ ID NO: 14. In some embodiments, the homology-directed repair template polynucleotide comprises SEQ ID NO: 9. In some embodiments, the point mutation comprises a N854K mutation. In some embodiments, the gRNA comprises SEQ ID NO: 15. In some embodiments, the homology-directed repair template polynucleotide comprises SEQ ID NO: 10. In some embodiments, the point mutation comprises a G598E mutation. In some embodiments, the gRNA comprises SEQ ID NO: 16. In some embodiments, the homology-directed repair template polynucleotide comprises SEQ ID NO: 11. In some embodiments, a proliferation rate of the edited and differentiated cell, is greater than a proliferation rate of an otherwise identical control iMGL, HPC or microglial precursor cell without the editing. In some embodiments, a transcriptional profile of microglia-related genes of the edited and differentiated iMGL more closely resembles a transcriptional profile of the microglia-related genes in a positive control iMGL or microglial precursor cell with exactly two native, wildtype CSF1R alleles compared to a transcriptomic profile of the microglia-related genes in an otherwise identical negative control iMGL comprising a mutant CSF1R gene. In some embodiments, the method for use further comprises transplanting the edited and differentiated iMGL, HPC or MPC into a brain of a subject. In some embodiments, the transplanting: increases number or density of Iba1-expressing edited and differentiated microglia in the brain; increases number, density, or average size of excitatory synapses in the brain; increases PSD95 or NSE expression in the brain; decreases accumulation of secreted osteopontin (OPN) in the brain; decreases frequency of axonal spheroids immunoreactive for neurofilament SMI312 and LAMP1 in the brain; decreases number, density, or frequency of hydroxyapatite calcium crystals; decreases levels of Tau phosphorylated at Thr217 (pTau217) in the brain; decreases GFAP expression levels in the brain; or decreases MCP-1 expression levels in the brain, wherein at least 6 weeks has passed since the transplantation of the edited and differentiated iMGL, HPC, microglial precursor cell, or myeloid cell into the subject. In some embodiments, the transplanting is autologous and the donor is the subject. In some embodiments, the transplanting is allogeneic and the donor is not the subject. In some embodiments, the edited and differentiated iMGL, HPC, or microglial precursor cell engrafts into and repopulates the brain of a subject with a leukodystrophy. In some embodiments, the edited and differentiated iMGL, HPC or microglial precursor cell is administered into the subject via a stereotactic, intraparenchymal, intracerebroventricular, intrathecal, subretinal, intraperitoneal, or intranasal injection. In some embodiments, the edited and differentiated iMGL, HPC or microglial precursor cell is administered into the subject via an intraperitoneal or intravenous injection.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Producing Human Microglia-Like Cells (iMGLs) from Induced Pluripotent Stem Cells (iPSCs)

A two-step, fully defined protocol was developed to successfully generate microglia-like cells (iMGLs) from iPSCs in just over five weeks. The methods and protocols may be used in like manner to generate iMGLs from other PSCs, including ESCs. This approach was utilized to successfully produce iMGLs from over 10 independent iPSC lines. First, iPSCs were differentiated into hematopoietic progenitors (iHPCs), which recapitulate microglia ontogeny as iHPCs represent early primitive hematopoietic cells derived from the yolk sac that give rise to microglia during development. This protocol yielded primitive iHPCs that were CD43+/CD235a+/CD41+ after 10 days. FACS sorting for CD43+ cells revealed that this approach produced iHPCs with a >90% purity.

Second, CD43+ iHPCs were grown in serum-free differentiation medium (formulated in house) containing CSF-1, IL-34, and TGFβ1. By day 14, cells expressed the myeloid-associated transcription factor PU.1 and the microglia-enriched protein TREM2 demonstrating an early commitment toward microglial fate. Because this protocol yielded large amounts of iMGLs, their development was followed by in vitro characterization every 4 days by flow cytometry. By day 14, early iMGLs were c-kit−/CD45+, suggesting commitment towards a myeloid lineage. Additionally, cells were further subdivided into CD45+/CX3CR1—(A1) and CD45+/CX3CR1+(A2) populations. CD45 expression was consistently monitored in developing iMGLs and compared to monocyte-derived macrophages (MD-Mφ). While CD45 expression increased with maturation, levels never reached that of macrophages, consistent with murine development. A small population of iMGLs (˜10%) also expressed intermediate CD11b levels by day 14 that also increased as cells matured, but again never reached macrophage levels.

By day 38, iMGLs exhibited high purity as assessed by purinergic receptor P2RY12 and TREM2 co-localization and quantification (>96%). One million iPSCs produced 30-40 million iMGLs with this protocol, suggesting that this approach can be readily scaled-up for high content screening. The resulting iMGLs resembled human microglia, but not monocytes or macrophages by cytospin/Giemsa Staining and protein expression. Like mouse microglia development in vivo, iMGLs developed in vitro expressed PU.1, TREM2, AXL, STAB1, P2RY6, CCR6, GPR84, Siglec11, Siglec12, P2RY12, P2RY13, OLFML3, Iba1, TMEM119, and CD11bint/CD45low, and resembled fetal microglia. As iMGLs matured in vitro they became more ramified, similar to microglia in vivo.

### Example 2: Transcriptome Analysis of iMGLs

The transcriptome of the iMGLs was profiled in comparison to human primary fetal microglia (Fetal MG) and adult microglia (Adult MG). The CD14+/CD16—monocytes (CD14 M), CD14+/CD16+ inflammatory monocytes (CD16 M), myeloid dendritic cells (Blood DCs), iHPCs, and iPSCs were also examined, in order to compare them to stem cells and other myeloid molecular signatures. Correlational analysis and Principal Component Analysis (PCA) revealed striking similarity of iMGLs to Fetal MG and Adult MG. Furthermore, the first principal component PC1 (21.3% variance) defined the differentiation time-series from iPSC through iHPC to iMGL cells while PC2 and PC3 defined the dendritic and monocyte trajectories, respectively.

Biclustering analysis using 300 microglial, macrophage, and other immune related genes adapted from previous studies identified similarities between groups and highlighted common gene clusters. This analysis again showed that iMGLs cluster with microglia but are distinct from other myeloid cells, iHPCs and iPSCs. Importantly, iMGLs, Fetal MG, and Adult MG expressed canonical microglial genes such as P2RY12, GPR34, C1Q, CABLES1, BHLHE41, TREM2, ITAM PROS1, APOE, SLCO2B1, SLC7A8, PPARD, and CRYBB1 (TABLE 1). When compared to monocytes, iMGLs expressed the myeloid genes, RUNX1, PU.1, and CSF1R, but did not express monocyte-specific transcription factors, IRF1, KLF4, NR4A1. Differential analysis between iMGLs, CD14 M, and CD16 M further emphasized that iMGLs predominantly expressed microglial genes (greater than two-fold change and p<0.001) including CX3CR1, TGFBR1, RGS10, and GAS6, but not monocyte and macrophage genes KLF2, TREM1, MPG, ITGAL, and ADGRE5. At the protein level, iMGLs, like primary microglia are CD45lo compared to CD45hi MD-Mφ, and express the microgglia surface proteins XCR1, TGFBR1, and PROS1. TABLE 2 and TABLE 3 show top GO pathways enriched in adult MG compared to fetal MG or iMGLs, respectively. TABLE 4 and TABLE 5 show GO pathways enriched in fetal MG compared to adult MG or iMGLs, respectively. TABLE 6 and TABLE 7 show GO pathways enriched in iMGLs compared to fetal MG or adult MG, respectively. Collectively, unbiased whole-transcriptome analysis strongly established iMGLs as a cell model that highly resembles primary human microglia.

TABLE 1
Biclustering analysis of microglial, macrophage, and other
immune related genes in iMGLs, microglia, and other cell types
GENES

| Comparisons | P2RY12 | GPR34 | CABLES1 | BHLHE41 | TREM2 | OLFML3 |
|---|---|---|---|---|---|---|
| CD14+ M VS. | 0.7965 | >0.9999 | >0.9999 | >0.9999 | 0.9987 | 0.9871 |
| CD16+ M | | | | | | |
| CD14+ M VS. | 0.7531 | 0.0063 | 0.5606 | >0.9999 | 0.6405 | >0.9999 |
| BLOOD DC | | | | | | |
| CD14+ M VS. | <0.0001 | <0.0001 | <0.0001 | 0.0064 | <0.0001 | <0.0001 |
| iMGL | | | | | | |
| CD14+ M VS. | <0.0001 | <0.0001 | <0.0001 | <0.0001 | <0.0001 | <0.0001 |
| FETAL MG | | | | | | |
| CD14+ M VS. | <0.0001 | <0.0001 | <0.0001 | <0.0001 | <0.0001 | <0.0001 |
| ADULT MG | | | | | | |
| CD16+ M VS. | 0.2047 | 0.0129 | 0.6603 | >0.9999 | 0.8586 | 0.9976 |
| BLOOD DC | | | | | | |
| CD16+ M VS. | <0.0001 | <0.0001 | <0.0001 | 0.0111 | <0.0001 | <0.0001 |
| iMGL | | | | | | |
| CD16+ M VS. | 0.0004 | <0.0001 | <0.0001 | <0.0001 | <0.0001 | <0.0001 |
| FETAL MG | | | | | | |
| CD16+ M VS. | 0.0002 | <0.0001 | <0.0001 | <0.0001 | <0.0001 | <0.0001 |
| ADULT MG | | | | | | |
| BLOOD DC VS. | <0.0001 | <0.0001 | <0.0001 | 0.0256 | <0.0001 | <0.0001 |
| iMGL | | | | | | |
| BLOOD DC VS. | <0.0001 | <0.0001 | <0.0001 | 0.0001 | <0.0001 | <0.0001 |
| FETAL MG | | | | | | |
| BLOOD DC VS. | <0.0001 | <0.0001 | <0.0001 | <0.0001 | <0.0001 | <0.0001 |
| ADULT MG | | | | | | |
| iMGL VS. | <0.0001 | >0.9999 | 0.9483 | 0.0256 | 0.0633 | <0.0001 |
| FETAL MG | | | | | | |
| iMGL VS. | <0.0001 | 0.8258 | 0.3015 | 0.0001 | 0.0633 | <0.0001 |
| ADULT MG | | | | | | |
| FETAL MG VS. | 0.9987 | 0.9431 | 0.1407 | 0.2995 | >0.999 | 0.9998 |
| ADULT MG | | | | | | |

| COMPARISONS | PROS1 | APOE | SLCO2B1 | SLC7A8 | PPARD | CRYBB1 |
|---|---|---|---|---|---|---|
| CD14+ M VS. | 0.8814 | >0.9999 | 0.9999 | >0.9999 | 0.4125 | 0.0011 |
| CD16+ M | | | | | | |
| CD14+ M VS. | 0.4077 | 0.9103 | 0.9994 | 0.9965 | 0.9185 | 0.6963 |
| BLOOD DC | | | | | | |
| CD14+ M VS. | <0.0001 | <0.0001 | <0.0001 | <0.0001 | <0.0001 | <0.0001 |
| iMGL | | | | | | |
| CD14+ M VS. | <0.0001 | <0.0001 | <0.0001 | <0.0001 | <0.0001 | <0.0001 |
| FETAL MG | | | | | | |
| CD14+ M VS. | <0.0001 | <0.0001 | <0.0001 | <0.0001 | <0.0001 | <0.0001 |
| ADULT MG | | | | | | |
| CD16+ M VS. | 0.9391 | 0.9455 | 0.9941 | 0.9987 | 0.138 | 0.0002 |
| BLOOD DC | | | | | | |
| CD16+ M VS. | <0.0001 | <0.0001 | <0.0001 | <0.0001 | <0.0001 | <0.0001 |
| iMGL | | | | | | |
| CD16+ M VS. | <0.0001 | <0.0001 | <0.0001 | <0.0001 | <0.0001 | <0.0001 |
| FETAL MG | | | | | | |
| CD16+ M VS. | <0.0001 | <0.0001 | <0.0001 | <0.0001 | <0.0001 | <0.0001 |
| ADULT MG | | | | | | |
| BLOOD DC VS. | <0.0001 | <0.0001 | <0.0001 | <0.0001 | <0.0001 | <0.0001 |
| iMGL | | | | | | |
| BLOOD DC VS. | <0.0001 | <0.0001 | <0.0001 | <0.0001 | 0.0004 | <0.0001 |
| FETAL MG | | | | | | |
| BLOOD DC VS. | <0.0001 | <0.0001 | <0.0001 | <0.0001 | <0.0001 | <0.0001 |
| ADULT MG | | | | | | |

| Comparisons | P2RY12 | GPR34 | CABLES1 | BHLHE41 | TREM2 | OLFML3 |
|---|---|---|---|---|---|---|
| iMGL VS. | 0.0008 | <0.0001 | 0.2803 | 0.0127 | 0.091 | >0.9999 |
| FETAL MG | | | | | | |
| iMGL VS. | 0.2533 | <0.0001 | 0.9909 | 0.4987 | 0.403 | <0.0001 |
| ADULT MG | | | | | | |
| FETAL MG VS. | 0.1787 | >0.9999 | 0.7213 | 0.4966 | 0.9658 | <0.0001 |
| ADULT MG | | | | | | |

TABLE 2
Top GO pathways enriched in adult MG compared to fetal MG
Adult MG vs Fetal MG

| | | LogP | Log |
|---|---|---|---|
| Description | GO ID | (—) | (q-value) |
| Extracellular matrix organization | 0030198 | 38.10 | −34.18 |
| Circulatory system development | 0072359 | 36.87 | −33.23 |
| Single organism cell adhesion | 0098602 | 32.24 | −29.04 |
| Regulation of nervous system | 0051960 | 28.56 | −25.55 |
| development | | | |
| Regulation of cellular component | 0051270 | 26.49 | −23.54 |
| movement | | | |
| Adaptive immune response | 0002250 | 25.87 | −22.96 |
| Response to cytokine | 0034097 | 20.02 | −17.49 |
| Epithelial cell proliferation | 0050673 | 19.25 | −16.74 |
| Central nervous system development | 0007417 | 19.12 | −16.63 |
| Negative regulation of cell | 0008285 | 18.82 | −16.34 |
| proliferation | | | |
| Tissue morphogenesis | 0048729 | 18.50 | −16.06 |
| Muscle structure development | 0061061 | 17.98 | −15.56 |
| Single organism cell adhesion | 0050808 | 17.86 | −15.48 |
| Regulation of nervous system | 0042063 | 17.26 | −14.91 |
| development | | | |
| Regulation of Growth | 0040008 | 16.62 | −14.32 |

TABLE 3
Top GO pathways enriched in adult MG compared to iMGL
Adult MG vs iMGL

| | | LogP | Log |
|---|---|---|---|
| Description | GO ID | (—) | (q-value) |
| Regulation of cell migration | 0030334 | 13.23 | −9.58 |
| Regulation of cell adhesion | 0030155 | 11.46 | −8.12 |
| Actin filament-based process | 0030029 | 11.03 | −7.78 |
| Regulation of anatomical structure | 0022603 | 10.95 | −7.74 |
| morphogenesis | | | |
| Cell junction organization | 0034330 | 10.29 | −7.12 |
| Enzyme linked receptor protein signaling | 0007167 | 9.73 | −6.76 |
| pathway | | | |
| Circulatory system development | 0072359 | 8.59 | −5.79 |
| Single-organism catabolic process | 0044712 | 8.13 | −5.42 |
| Oxidation-reduction process | 0055114 | 7.78 | −5.11 |
| Plasma membrane organization | 0007009 | 7.35 | −4.76 |
| Cellular response to oxygen-containing | 1901701 | 7.31 | −4.73 |
| compound | | | |
| Negative regulation of cell proliferation | 0008285 | 7.28 | −4.72 |
| Positive regulation of phosphorylation | 0042327 | 7.14 | −4.63 |
| Renal system development | 0072001 | 6.77 | 4.30 |

TABLE 4
GO pathways enriched in fetal MG compared to adult MG
Fetal vs Adult MG

| | | LogP | Log |
|---|---|---|---|
| Description | GO ID | (—) | (q-value) |
| Leukocyte chemotaxis | 0030595 | 6.92 | −3.07 |
| Response to acidic pH | 0010447 | 4.07 | −1.33 |
| Inorganic ion homeostasis | 0098771 | 4.04 | −1.31 |
| Regulation of cell migration | 0030334 | 3.80 | −1.18 |
| Circulator system process | 0003013 | 3.72 | −1.13 |
| Melanosome organization | 0032438 | 3.70 | −1.13 |
| Anion transport | 0006820 | 3.54 | −1.01 |
| Macrophage migration | 1905517 | 3.47 | −0.95 |
| Transmembrane receptor protein tyrosine | 0007169 | 3.23 | −0.81 |
| kinase signaling pathway | | | |
| Negative regulation of receptor activity | 2000272 | 3.14 | −0.76 |
| Positive regulation of phagocytosis, | 0060100 | 3.13 | −0.76 |
| engulfment | | | |
| Sterol import | 0035376 | 3.13 | −0.76 |
| Behavior | 0007610 | 3.11 | −0.75 |
| Positive regulation of transport | 0051050 | 3.02 | −0.70 |
| Vesicle organization | 0016050 | 2.96 | −0.65 |

TABLE 5
GO pathways enriched in fetal MG compared to iMGLs
Fetal MG vs. iMGL

| | | LogP | Log |
|---|---|---|---|
| Description | GO ID | (—) | (q-value) |
| Single-organism catabolic process | 0044712 | 10.41 | −6.64 |
| Regulation of cell migration | 0030334 | 9.90 | −6.26 |
| Iron ion transport | 0006826 | 8.68 | −5.52 |
| Divalent metal ion transport | 0070838 | 8.44 | −5.34 |
| Carbohydrate metabolic process | 0005975 | 7.39 | −4.59 |
| Small GTPase mediated signal | 0007264 | 6.69 | −3.99 |
| transduction | | | |
| Angiogenesis | 0001525 | 6.64 | −3.97 |
| Positive regulation of transport | 0051050 | 6.15 | −3.59 |
| Positive regulation of intracellular signal | 1902533 | 6.12 | −3.57 |
| transduction | | | |
| Aminoglycan metabolic process | 0006022 | 6.03 | −3.50 |
| Cell projection assembly | 0030031 | 5.81 | −3.31 |
| Cell-substrate adhesion | 0031589 | 5.68 | −3.21 |

TABLE 6
Top GO pathways enriched in iMGLs compared to fetal MG
iMGL vs. Fetal MG

| | | LogP | Log |
|---|---|---|---|
| Description | GO ID | (—) | (q-value) |
| Single organism cell adhesion | 0098602 | 30.48 | −26.23 |
| Mitotic cell cycle process | 1903047 | 21.34 | −17.87 |
| Immune system development | 0002520 | 18.56 | −15.55 |
| Regulation of cellular component | 0051270 | 16.11 | −13.32 |
| movement | | | |
| Mitotic cell cycle phase transition | 0044772 | 15.94 | −13.18 |
| Leukocyte migration | 0050900 | 15.19 | −12.49 |
| Taxis | 0042330 | 14.76 | −12.12 |
| Positive regulation of cell differentiation | 0045597 | 14.76 | −12.12 |
| Inflammatory response | 0006954 | 14.64 | −12.03 |
| Anatomical structure formation involved | 0048646 | 13.83 | −11.27 |
| in morphogenesis | | | |
| Positive regulation of cell proliferation | 0009294 | 13.82 | −11.26 |
| Positive regulation of intracellular signal | 1902533 | 12.20 | −9.73 |
| transduction | | | |
| Positive regulation of hydrolase activity | 0051345 | 11.80 | −9.35 |
| Negative regulation of multicellular | 0051241 | 11.67 | −9.23 |
| organismal process | | | |
| Negative regulation of cell proliferation | 0008285 | 11.62 | −9.18 |

TABLE 7
Top GO pathways enriched in iMGLs compared to adult MG
iMGL vs. Adult MG

| | | LogP | Log |
|---|---|---|---|
| Description | GO ID | (—) | (q-value) |
| Mitotic cell cycle process | 1903047 | 28.46 | −24.22 |
| Regulation of cell migration | 0051726 | 19.26 | −15.94 |
| DNA replication | 0006260 | 17.22 | −14.09 |
| Chromatin organization | 0006325 | 11.44 | −8.64 |
| Regulation of nuclear division | 0051783 | 11.25 | −8.47 |
| Immune system development | 0002520 | 10.90 | −8.15 |
| DNA-dependent DNA replication | 0006261 | 10.22 | −7.51 |
| Negative regulation of transcription from | 0000122 | 10.20 | −7.50 |
| RNA polymerase II promoter | | | |
| Microtubule cytoskeleton organization | 0000226 | 9.96 | −7.27 |
| Leukocyte activation | 0045321 | 9.57 | −6.94 |
| Regulation of small GTPase mediated | 0051056 | 9.16 | −6.56 |
| signal transduction | | | |
| Cell cycle G2/M phase transition | 0044839 | 9.01 | −6.44 |
| Signal transduction by p53 class mediator | 0072331 | 8.47 | −5.99 |
| Regulation of transcription involved in | 0000083 | 8.34 | −5.88 |
| G1/S transition of mitotic cell cycle | | | |
| Cellular response to oxygen-containing | 1901701 | 8.15 | −5.73 |
| compound | | | |

### Example 3: Genetic Correction of CSF1R1 Mutations

Genetic correction of CSF1R mutations in iPSCs derived from ALSP patient fibroblasts was achieved using Clustered Regularly Interspaced Short Palindromic Repeats with CRISPR associated endonuclease 9 (CRISPR/Cas9) via two distinct methods. In the first, hCSF1R coding sequences (CDS) were inserted into the second exon of CSF1R. In the second, individual patient-specific single nucleotide polymorphisms (SNPs) were corrected by homology-directed repair with a single-strand oligodeoxyribonucleotide (ssODN) template.

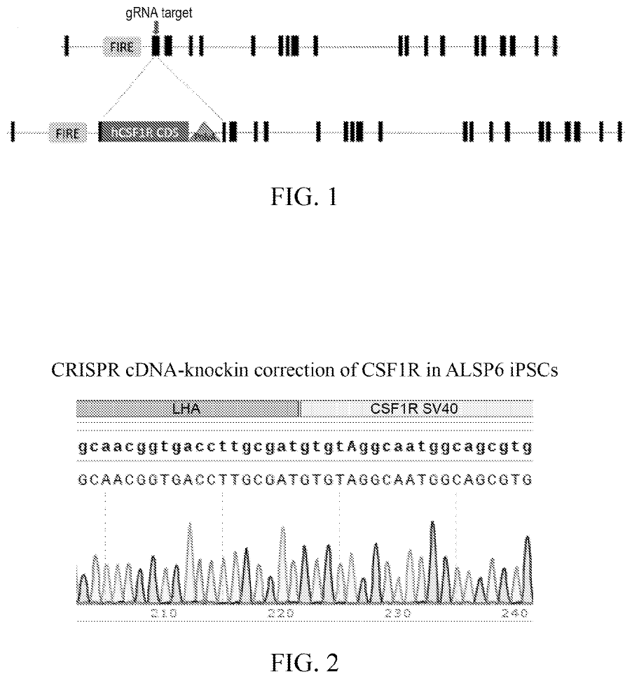

The second exon of CSF1R lies immediately 3′ of the fms-intronic regulatory element (FIRE) (see FIG. 1) and was selected as the insertion site to keep the FIRE intact, as FIRE sequences are necessary for proper hCSF1R expression in microglia (Rojo et al., 2019). For insertion of the coding sequence, a plasmid template was constructed containing the WI human CSF1R coding nucleic acid sequence (cDNA) of SEQ ID NO: 2 and a SV40 poly-A signal (SEQ ID NO: 6 and within SEQ ID NO: 2) at the 3′ end of the CSF1R cDNA fragment to prevent transcription of the remaining endogenous CSF1R gene. The plasmid homology repair template contains the WT CSF1R coding nucleic acid sequence (cDNA) of SEQ ID NO: 2 flanked on each side by 1 kbp right (3′) homology arm (SEQ ID NO: 4) and 1 kbp left (5′) homology arm (SEQ ID NO: 3) immediately adjacent to the target site. A silent mutation (V43V; nucleotide position 5 of SEQ ID NO: 2) is introduced to prevent recutting of correctly edited alleles by the Cas9 endonuclease and increasing the efficiency of homozygous knock-in insertion of the WT CSF1R coding sequence. Following Cas9 targeting by a sgRNA and Cas9-mediated DNA cutting to form double-stranded breaks in the target CSF1R gene, homology-directed repair mechanisms facilitate the insertion of the CSF1R cDNA sequence (SEQ ID NO: 2) into the target insertion site within exon 2 of the human CSF1R gene. The sequences of the guide RNA, left homology arm, CSF1R CDS, and right homology are used in this method are listed in TABLE 8. A polyadenylation sequence at the 3′ end of the inserted CSF1R coding sequence terminates transcription, preventing expression of the endogenous mutant CSF1R sequence. The CSF1R CDS does not include the full CDS; the CDS of the inserted cDNA fragment spans exons 2-21 of the CSF1R CDS and lacks the start codon. A DNA sequence chromatogram demonstrating successful insertion of the CSF1R CDS at the expected site in iPSCs derived from the ALSP6 cell line is shown in FIG. 2. It is important to introduce the CSF1R coding sequence after the FIRE, which is necessary to turn on the production of the downstream CSF1R coding sequence within microglia.

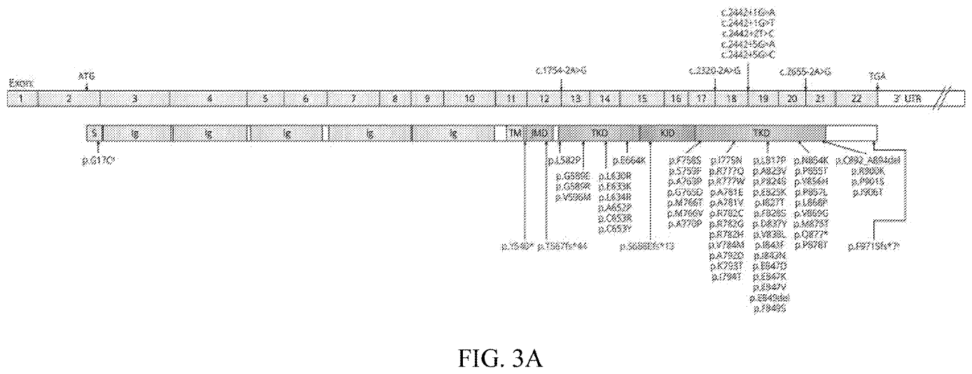

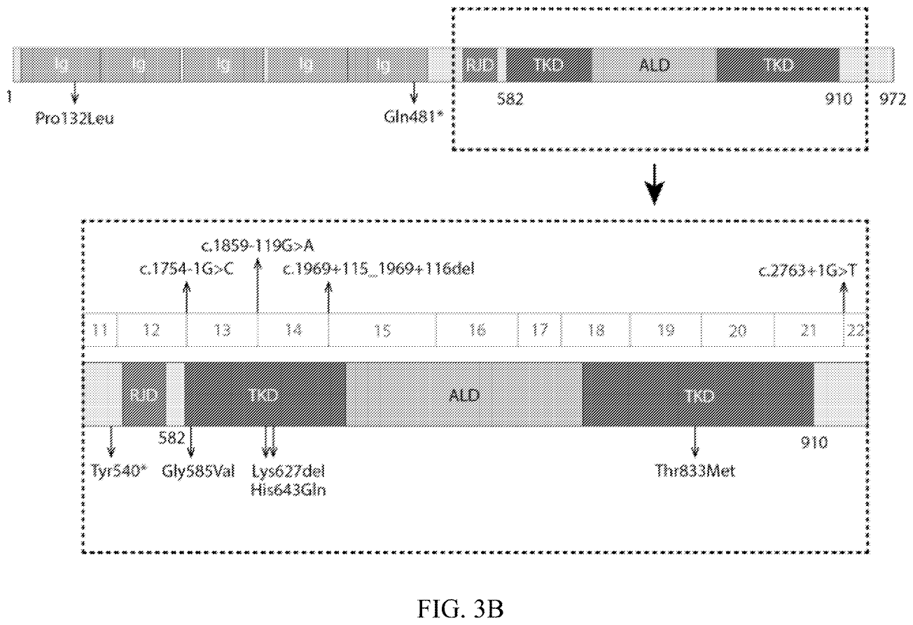

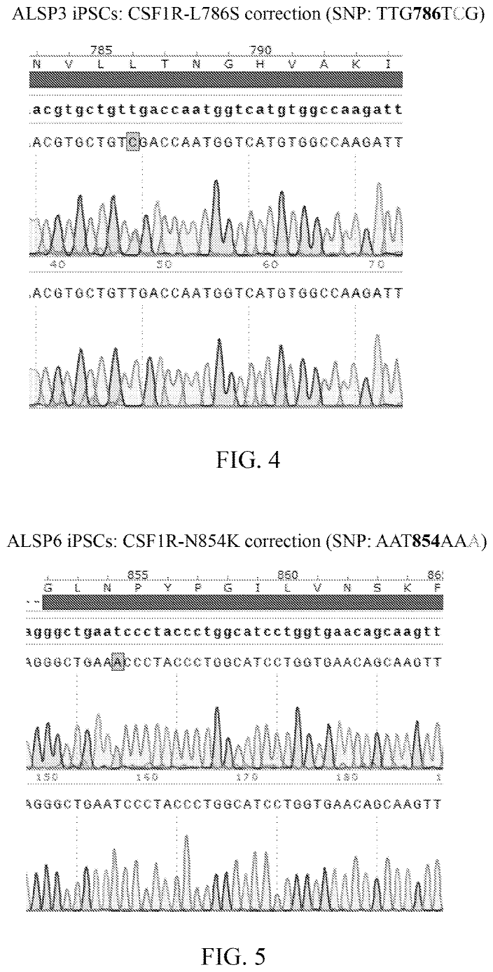

Individual patient-specific single nucleotide polymorphisms (SNPs) of the human CSF1R gene are primarily located between exons 13 to 21 (FIG. 3A-3B). The SNPs in six ALSP cell lines are depicted in TABLE 9. These SNPs were corrected using the single-stranded oligodeoxyribonucleotide (ssODN) templates depicted in TABLE 10 and the guide RNAs depicted in TABLE 11. DNA sequence chromatograms demonstrating successful correction of CSF1R point mutations in two CSF1R cell lines are shown in FIG. 4 and FIG. 5. Briefly, a 100-nucleotide ssODN was individually designed and created for the correction of each single nucleotide polymorphism (SNP) in hCSF1R. Following Cas9 targeting and double-stranded cutting of the target SNP, the ssODN binds to the complementary strand of DNA via homology-directed repair mechanisms. Importantly, the gRNA and ssODN were designed to specifically target and cut the defective hCSF1R allele containing the SNP and to avoid altering the WT CSF1R allele.

### Gene-Editing Methods

First, the gRNA was assembled by annealing 200 μM crRNA (CRISPR RNA) and 200 μM tracrRNA (trans-activating crRNA) in duplex buffer (IDTDNA) on a heating block at 95° C. for 5 min. 50 μg of Alt-R® S.p. HiFi Cas9 Nuclease V3 (IDTDNA; Vakulskas et al., 2018) was combined with the resulting gRNA for 15 min at room temperature (RT) to form the ribonucleoprotein (RNP) complex. Next, 2×10⁵ iPSCs were isolated following Accutase (Gibco) enzymatic digestion of adhered colonies for 3 min at 37° C. iPSCs are resuspended in 100 μL nucleofection buffer from Human Stem Cell Nucleofector™ Kit 2 (Lonza) and combined with 1 μg plasmid template (Vectorbuilder) or 2 uM ssODN Template (IDTDNA) and previously formed RNP complex. The suspension was transferred to the Amaxa Nucleofector cuvette and transfected using program B-016. Cells were plated in mTeSR™ Plus (STEMCELL Technologies) media with 0.25 μM Thiazovivin (STEMCELL Technologies) and CloneR™ (STEMCELL Technologies) supplement overnight to recover. Adhered cells were enzymatically digested with Accutase at 37° C. and mechanically single-cell plated using a multichannel pipette on Vitronectin coated 96-well plates in mTeSR™ Plus media with 0.25 μM Thiazovivin and CloneR™ for clonal isolation and expansion. Plates were incubated in culture at 37° C. and 5% CO2. Starting at day 5, plates were visually scanned to identify wells of single clone expanding colonies. Identified colonies were cultured with mTeSR™ Plus media with daily media changes. On day 15, colonies were passaged to vitronectin-coated 48-well plates for expansion and genomic analysis. At time of passaging, a sample per colony was collected separately in an 8-strip tube from which Genomic DNA was extracted using Extracta DNA prep for polymerase chain reaction (PCR; Quantabio) using Taq PCR Master Mix (ThermoFisher Scientific) at the cut site. PCR products were sent for sequencing and interpreted to identify properly edited clones for expansion and differentiation.

### Quality Control of Gene Editing

Following genetic correction of CSF1R, iPSC lines underwent a series of quality control steps. Initially, the top-five off-target sites of the gRNA used to knock in the CDS into the second exon of CSF1R, as identified by bioinformatic analysis (IDTDNA), were amplified by PCR and Sanger Sequenced to confirm no aberrant alterations resulted from the RNP complex. Unmodified lines underwent karyotyping and copy number variation (CNV) analysis by array-based comparative genomic hybridization (aCGH; Cell Line Genetics) to ensure additional genomic alterations were not inherited in the generation of the corrected line as compared to the parental line. Lastly, safety testing after genetic correction was done including sterility, mycoplasma, and bacterial endotoxins using compendial methods.

Differentiation of iPSCs to HPCs

iPSCs were first differentiated into CD43+ primitive HPCs using a STEMdiff™ Hematopoietic Kit (Stem Cell Technologies).

On Day-1, feeder-free iPSCs that were expanded in mTeSR™ Plus (STEMCELL Technologies) media were passaged with ReLeSR™, into mTeSR™ Plus (STEMCELL Technologies) media with 0.25 μM Thiazovivin, onto Vitronectin coated (1 mg/mL) 6-well plates. 80-100 aggregates (cell clumps of 50-200 cells and a diameter of 100-200 μm) were plated per well.

To ensure the optimal plating density, iPSCs were plated at 2-3 different densities, and the wells with optimal density were then selected to proceed with on Day 0.

When approximately two 100 cell colonies per cm² had been achieved, the mTeSR™ Plus (STEMCELL Technologies) media was replaced with media A (Basal media plus Supplement A at 1:200 dilution). On Day 2 (48 hours after original media change), 50% of media A was replaced.

On Day 4, all media was removed, and media B (Basal media plus supplement B at 1:200) was added.

On Days 6 and 8, media B was supplemented (without removing media).

On Days 10 and 12, non-adherent cells were collected. To maintain purity, media with non-adherent cells was carefully removed (without washing cells off wells as this would loosen cells, which were not CD43+, and decrease culture purity) and centrifuged at 300×G for 5 min. After centrifugation, the supernatant (conditioned media) was taken and added back to each well. Fresh media B was also added to each well.

The non-adherent cells represented highly pure populations (>93%) of CD43+ hematopoietic progenitor cells when analyzed by fluorescence-activated cell sorting (FACS) analysis. Collection of floating cells has been shown to be sufficient to isolate large numbers of highly purified CD43+ cells. No FACS or magnetic-activated cell sorting isolation was required as identical microglia were produced using any of these methods (McQuade et al., 2018).

Differentiation of CD43+ HPCs to iPSC-Derived Microglia

HPCs were transferred directly into microglial differentiation media (DMEM/F12, 2× insulin-transferrin-selenium, 2× B27, 0.5× N2, 1× Glutamax, 1× non-essential amino acids, 400 μM monothioglycerol, 5 gg/mL insulin) supplemented immediately before use with 100 ng/mL of interleukin-34 (IL-34), 50 ng/mL of transforming growth factor beta 1 (TGF-β1), and 25 ng/mL of macrophage colony-stimulating factor (M-CSF). The cells were plated onto 1 mg/mL Vitronectin-coated 6-well plates at 80,000 to 120,000 cells per well. Throughout the differentiation of HPCs to microglia, the cells were grown predominantly non-adherently.

On Days 2, 4, 6, 8 and 10, fresh media supplemented with freshly thawed tri-cytokine cocktail (100 ng/mL of IL-34. 50 ng/mL of TGF-β1 and 25 ng/mL of M-CSF) was added. The media was not fully removed during microglial differentiation as the cells secreted paracrine cytokine signals and would not properly differentiate upon removal of the media.

On Day 12, 6 mL media was collected from each 35 mm well, leaving 1 mL of conditioned media on the plate. The non-adherent cells from the collected media were then centrifuged at 300×G for 5 minutes. The media was aspirated, and the non-adherent cells were re-suspended in fresh media containing the tri-cytokine cocktail, before being added back to the same wells containing the 1 mL conditioned media.

On Days 14, 16, 18, 20, 22 and 24, the cells were fed with 1 mL of base media containing the tri-cytokine cocktail (100 ng/mL of IL-34, 50 ng/mL of TGF-β1 and 25 ng/mL of M-CSF).

On Day 25, the cells were harvested as drug product (DP).

The DP was manually filled into vials (4×10⁶ cells in 1 mL BAMBANKER™ serum-free media), cryopreserved in a controlled-rate freezer, and stored in the vapor phase of a liquid nitrogen freezer at <−150° C. The frozen DP was then shipped to clinical sites in dry shippers jacketed in liquid nitrogen at a temperature of <−150° C.

### Thaw and Recovery of Frozen DP

On Day 0, cells were thawed in a 37° C. water bath then transferred to a 15 mL tube, centrifuged, then resuspended in basal media containing the tri-cytokine cocktail. The cells were seeded in a Vitronectin-coated plate and incubated in a humidified incubator (5% C02, 37° C.). Media exchange was performed approximately 24 hours post-seed and approximately every 48 hours thereafter by adding basal media containing the tri-cytokine cocktail. The seeded microglia were recovered in culture for a total of 5 days.

### Harvest and Formulation of DP for Transplantation

The recovered NGL-101 DP was harvested and washed with basal media, then washed and resuspended in DPBS for cell counting and assessment of viability. After counting, the cells were formulated at a concentration of 62.5×10³ viable cells/L in DPBS for transplantation.

TABLE 8
Polynucleotides for CRISPR-guided Insertion of CSF1R cDNA downstream of FIRE in
ALSP Patient iPSC Lines - ALSP6 cDNA knock-in

| SEQ | | |
|---|---|---|
| ID | Descrip- | |
| NO: | tion | Nucleic Acid Sequence |
| 1 | CSF1R | MRHTNYSFSPWHGFTIHRAKFIQSQDYQCSALMGGRKV |
| | amino | MSISIRLKVQKVIPGPPALTLVPAELVRIRGEAAQIVCSAS |
| | acid | SVDVNFDVFLQHNNTKLAIPQQSDFHNNRYQKVLTLNL |
| | sequence | DQVDFQHAGNYSCVASNVQGKHSTSMFFRVVESAYLN |
| | | LSSEQNLIQEVTVGEGLNLKVMVEAYPGLQGFNWTYLG |
| | | PFSDHQPEPKLANATTKDTYRHTFTLSLPRLKPSEAGRY |
| | | SFLARNPGGWRALTFELTLRYPPEVSVIWTFINGSGTLLC |
| | | AASGYPQPNVTWLQCSGHTDRCDEAQVLQVWDDPYPE |
| | | VLSQEPFHKVTVQSLLTVETLEHNQTYECRAHNSVGSGS |
| | | WAFIPISAGAHTHPPDEFLFTPVVVACMSIMALLLLLLLL |
| | | LLYKYKQKPKYQVRWKIIESYEGNSYTFIDPTQLPYNEK |
| | | WEFPRNNLQFGKTLGAGAFGKVVEATAFGLGKEDAVL |
| | | KVAVKMLKSTAHADEKEALMSELKIMSHLGQHENIVNL |
| | | LGACTHGGPVLVITEYCCYGDLLNFLRRKAEAMLGPSL |
| | | SPGQDPEGGVDYKNIHLEKKYVRRDSGFSSQGVDTYVE |
| | | MRPVSTSSNDSFSEQDLDKEDGRPLELRDLLHFSSQVAQ |
| | | GMAFLASKNCIHRDVAARNVLLTNGHVAKIGDFGLARD |
| | | IMNDSNYIVKGNARLPVKWMAPESIFDCVYTVQSDVWS |
| | | YGILLWEIFSLGLNPYPGILVNSKFYKLVKDGYQMAQPA |
| | | FAPKNIYSIMQACWALEPTHRPTFQQICSFLQEQAQEDR |
| | | RERDYTNLPSSSRSGGSGSSSSELEEESSSEHLTCCEQGDI |
| | | AQPLLQPNNYQFC |
| 2 | CSF1R cDNA | gtgtAggcaatggcagcgtggaatgggatggccccccatcacctcactggaccctgta |
| | Silent A | ctctgatggctccagcagcatcctcagcaccaacaacgctaccttccaaaacacgggga |
| | insertion | cctatcgctgcactgagcctggagaccccctgggaggcagcgccgccatccacctctat |
| | to | gtcaaagaccctgcccggccctggaacgtgctagcacaggaggtggtcgtgttcgagg |
| | prevent | accaggacgcactactgccctgtctgctcacagacccggtgctggaagcaggcgtctcg |
| | recutting | ctggtgcgtgtgcgtggccggcccctcatgcgccacaccaactactccttctcgccctgg |
| | by Cas9 at | catggcttcaccatccacagggccaagttcattcagagccaggactatcaatgcagtgcc |
| | nucleotide | ctgatgggtggcaggaaggtgatgtccatcagcatccggctgaaagtgcagaaagtcat |
| | 5; | cccagggcccccagccttgacactggtgcctgcagagctggtgcggattcgaggggag |
| | CSF1R | gctgcccagatcgtgtgctcagccagcagcgttgatgttaactttgatgtcttcctccaaca |
| | coding | caacaacaccaagctcgcaatccctcaacaatctgactttcataataaccgttaccaaaaa |
| | sequence: | gtcctgaccctcaacctcgatcaagtagatttccaacatgccggcaactactcctgcgtgg |
| | Stop codon | ccagcaacgtgcagggcaagcactccacctccatgttcttccgggtggtagagagtgcct |
| | at 2793 | acttgaacttgagctctgagcagaacctcatccaggaggtgaccgtgggggaggggctc |
| | SV40 | aacctcaaagtcatggtggaggcctacccaggcctgcaaggttttaactggacctacctg |
| | poly-A | ggacccttttctgaccaccagcctgagcccaagcttgctaatgctaccaccaaggacaca |
| | signal | tacaggcacaccttcaccctctctctgccccgcctgaagccctctgaggctggccgctact |
| | 2813-3034 | ccttcctggccagaaacccaggaggctggagagctctgacgtttgagctcacccttcgat |
| | | accccccagaggtaagcgtcatatggacattcatcaacggctctggcacccttttgtgtgc |
| | | tgcctctgggtacccccagcccaacgtgacatggctgcagtgcagtggccacactgata |
| | | ggtgtgatgaggcccaagtgctgcaggtctgggatgacccataccctgaggtcctgagc |
| | | caggagcccttccacaaggtgacggtgcagagcctgctgactgttgagaccttagagca |
| | | caaccaaacctacgagtgcagggcccacaacagcgtggggagtggctcctgggccttc |
| | | atacccatctctgcaggagcccacacgcatcccccggatgagttcctcttcacaccagtg |
| | | gtggtcgcctgcatgtccatcatggccttgctgctgctgctgctcctgctgctattgtacaag |
| | | tataagcagaagcccaagtaccaggtccgctggaagatcatcgagagctatgagggca |
| | | acagttatactttcatcgaccccacgcagctgccttacaacgagaagtgggagttccccc |
| | | ggaacaacctgcagtttggtaagaccctcggagctggagcctttgggaaggtggtggag |
| | | gccacggcctttggtctgggcaaggaggatgctgtcctgaaggtggctgtgaagatgct |
| | | gaagtccacggcccatgctgatgagaaggaggccctcatgtccgagctgaagatcatga |
| | | gccacctgggccagcacgagaacatcgtcaaccttctgggagcctgtacccatggagg |
| | | ccctgtactggtcatcacggagtactgttgctatggcgacctgctcaactttctgcgaagga |
| | | aggctgaggccatgctgggacccagcctgagccccggccaggaccccgagggaggc |
| | | gtcgactataagaacatccacctcgagaagaaatatgtccgcagggacagtggcttctcc |
| | | agccagggtgtggacacctatgtggagatgaggcctgtctccacttcttcaaatgactcctt |
| | | ctctgagcaagacctggacaaggaggatggacggcccctggagctccgggacctgctt |
| | | cacttctccagccaagtagcccagggcatggccttcctcgcttccaagaattgcatccacc |
| | | gggacgtggcagcgcgtaacgtgctgttgaccaatggtcatgtggccaagattggggac |
| | | ttcgggctggctagggacatcatgaatgactccaactacattgtcaagggcaatgcccgc |
| | | ctgcctgtgaagtggatggccccagagagcatctttgactgtgtctacacggttcagagc |
| | | gacgtctggtcctatggcatcctcctctgggagatcttctcacttgggctgaatccctaccct |
| | | ggcatcctggtgaacagcaagttctataaactggtgaaggatggataccaaatggcccag |
| | | cctgcatttgccccaaagaatatatacagcatcatgcaggcctgctgggccttggagccc |
| | | acccacagacccaccttccagcagatctgctccttccttcaggagcaggcccaagagga |
| | | caggagagagcgggactataccaatctgccgagcagcagcagaagcggtggcagcg |
| | | gcagcagcagcagtgagctggaggaggagagctctagtgagcacctgacctgctgcg |
| | | agcaaggggatatcgcccagcccttgctgcagcccaacaactatcagttctgctgagctt |
| | | cgagcagacatgacagacatgataagatacattgatgagtttggacaaaccacaactaga |
| | | atgcagtgaaaaaaatgctttatttgtgaaatttgtgatgctattgctttatttgtaaccatta |
| | | taagctgcaataaacaagttaacaacaacaattgcattcattttatgtttcaggttcaggggga |
| | | ggtgtgggaggttttttaaagcaagtaaaacctctacaaatgtggtactgattttgttgcgt |
| 3 | Left | Gcttgaatcagctctcacttccctcctttgcccctattttaggccctggaaaaatgctgacg |
| | homology | ctgcagaggcaacgggccttcttcccggacagcctgataggggtttcaagttctcttttctc |
| | arm; | cttcaagaaaattttccttaaaagagattggcttcccagtaaacacagatgtgtgggggtgc |
| | gRNA binds | cggggtgagctgctgggtgtagactaggtaataaacatagtgactaactcttactgagcca |
| | nucleotides | tttatttggtgacaggtgatgttctaaagtcttcccatgcatttaaaatgcctaacataccaat |
| | 984-1000 | gagtgggtacgatgattgtccctgttttataggtggggaaactgaggcatggcacctcccc |
| | | atcccactgtgctgcagaccagatgtccattggtgggaggggcacaccaggagattctt |
| | | gggacctctctaactctgctgggctaagatcctacatctctttttttttctttcccatatgaat |
| | | taagctgaggacttggccgtgaacattccattcatttgtttcttcattcggtggtagaaatacg |
| | | tccactttgtacacagggttaaaagagtccattcctggggagtagaaagatggcatcacagc |
| | | agggaagactgaggcaggaggctgaggaccccagggggacagaggcctgggtgag |
| | | aggctgagcaagctgcaagccccctttctcagaggagggacctcctggacatcagaga |
| | | catcagtctgtccctgagcaggttgagggttaggagctgagcaaatgaccagggggcag |
| | | gggctcgttcaaggtggtcccttgatggcacagcaccatccctgccaagctaccacccat |
| | | ctcagagtcaggacggcccaaggggcgcatcctagacctcacttctgtctgctgtccctct |
| | | ctcccaccaggtcagggaatcccagtgatagagcccagtgtccctgagctggtcgtgaa |
| | | gccaggagcaacggtgaccttgcgat |
| 4 | Right | Gtgtgggcaatggcagcgtggaatgggatggccccccatcacctcactggaccctgta |
| | homology | ctctgatggctccagcagcatcctcagcaccaacaacgctaccttccaaaacacgggga |
| | arm | cctatcgctgcactgagcctggagaccccctgggaggcagcgccgccatccacctctat |
| | | gtcaaaggtgaggagtctgagcctcctcccaagaggcctgacccggcaggccccacta |
| | | caatgggccctaaaattaacaatcgtaacaattcagctctgcatttactgagtgctggctat |
| | | gagcaaggacctggaagagctgctaatgtaatgcagtcctcacaacaaccctgcaagtc |
| | | gggtctatgatgatgcattttctagaagtgcagggaggttatccaaggtcacacagcctca |
| | | catagtgggactagactggagcccaggtgcgcctgactctggagccaccacgctgaag |
| | | catccgctgaactgtcctggcgtggtgtgacctcagatgaatgatcagcctctctgagcttc |
| | | cttgtcacctatgtccaggtactccttggcccagtggagggagggcagttgtaaccctgtg |
| | | ccctcctctactctagaccctgcccggccctggaacgtgctagcacaggaggtggtcgtg |
| | | ttcgaggaccaggacgcactactgccctgtctgctcacagacccggtgctggaagcagg |
| | | cgtctcgctggtgcgtgtgcgtggccggcccctcatgcgccacaccaactactccttctc |
| | | gccctggcatggcttcaccatccacagggccaagttcattcagagccaggactatcaatg |
| | | cagtgccctgatgggtggcaggaaggtgatgtccatcagcatccggctgaaagtgcaga |
| | | aaggtgcgtggggcatggggaccggcagccaggcctgaagagtggggacagagagc |
| | | cggcggccacatgggtggtgactggggactgggtgtgatggggggcagt |
| 5 | gRNA | AACGGUGACCUUGCGAUGUG |
| 6 | SV40 | cagacatgataagatacattgatgagtttggacaaaccacaactagaatgcagtgaaaaa |
| | poly-A | aatgctttatttgtgaaatttgtgatgctattgctttatttgtaaccattataagctgcaataa |
| | tail of | acaagttaacaacaacaattgcattcattttatgtttcaggttcagggggaggtgtgggaggttt |
| | CSFIR | tttaaagcaagtaaaacctctacaaatgtggta |
| | cDNA | |

TABLE 9
CSF1R Correction Strategy of Point
Mutations in ALSP Patient iPSC Lines

| ALSP Patient | Amino Acid | DNA mutation |
|---|---|---|
| iPSC line | Mutation | (SNP) |
| ALSP 2 | M875I | ATG875ATT |
| ALSP 3 | L786S | TTG786TCG |
| ALSP 4 & 5 | M875T | ATG875ACG |
| ALSP 6 | N854K | AAT854AAA |
| ALSP 7 | G589E | GGA589GAA |

TABLE 10
Single-stranded oligodeoxynucleotides for
correcting point mutations in ALSP Patient
iPSC Lines

| | ALSP | |
|---|---|---|
| SEQ | Patient | |
| ID | iPSC | Nucleic |
| NO: | line | Acid Sequence of ssODN |
| 7 | ALSP | ccctgggatcccttcgcttacatattcttt |
| | 2 | ggggcaaatgcaggctgggccatttggtat |
| | | ccatccttcaccagtttatagaacttgctg |
| | | ttcaccagga |
| 8 | ALSP | gtgctttccctcagtgcatccaccgggacg |
| | 3 | tggcagcgcgtaacgtgctgttgaccaatg |
| | | gtcatgtggccaagattggggacttcgggc |
| | | tggctagggacatcatgaat |
| 9 | ALSP | ccctgggatcccttcgcttacatattcttt |
| | 4 & 5 | ggggcaaatgcaggctgggccatttggtat |
| | | ccatccttcaccagtttatagaacttgctg |
| | | ttcaccagga |
| 10 | ALSP | agacaatgggcatggcctcttacctgatct |
| | 6 | tggcctttgcagggctgaatccctaccctg |
| | | gcatcctggtgaacagcaagttctataaac |
| | | tggtgaaggatggataccaa |
| 11 | ALSP | ggtgcccagggacttaagggacctgtgtgc |
| | 7 | gtggcaggtaagaccctcggagctggagcc |
| | | tttgggaaggtggtggaggccacggccttt |
| | | ggtctgggca |

TABLE 11
CRISPR gRNAs for correcting point
mutations in ALSP Patient iPSC Lines

| | ALSP | |
|---|---|---|
| SEQ | Patient | Nucleic Acid |
| ID | iPSC | Sequence encoding |
| NO: | line | CRISPR gRNA |
| 12 | ALSP 2 | CAAAUGCAGGCUGGGCAAUU |
| 13 | ALSP 3 | CGUAACGUGCUGUCGACCAA |
| 14 | ALSP 4 & 5 | CAAAUGCAGGCUGGGCCGUU |
| 15 | ALSP 6 | GCAGGGCUGAAACCCUACCC |
| 16 | ALSP 7 | GCAGGUAAGACCCUCGAAGC |

### Example 4: The Csf1rΔFIRE/ΔFIRE Mouse Model for ALSP

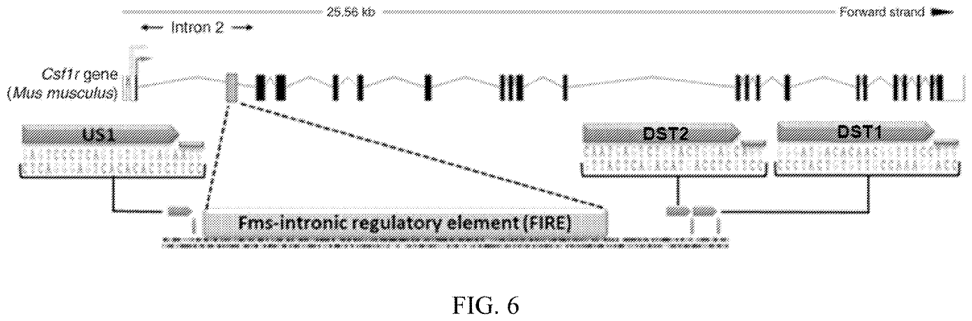

The Csf1rΔFIRE/ΔFIRE mouse model was generated by microinjecting vectors encoding the FIRE upstream gRNA (US1), downstream gRNA (DST1), downstream gRNA (DST2), and Cas9 (see FIG. 6) in to C57BL/6J/CBA F1 mouse oocytes. Rojo et. al., Nat Commun. 2019 Jul. 19; 10(1):3215) One male founder with the expressed 418 bp deletion was then crossed with Csf1r+/+ C57BL/6J females and the progeny were interbred.

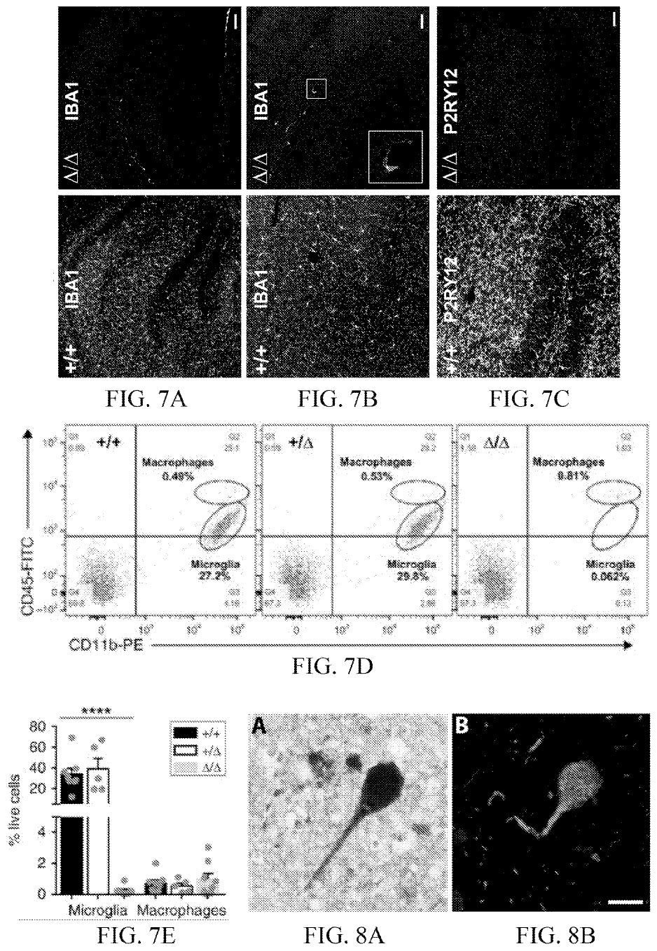

Genomic deletion of FIRE in mice selectively impacts CSF1R expression and macrophage development in specific tissues. Csf1rΔFIRE/ΔFIRE mice lack embryonic macrophages as well as resident macrophages in the brain (see FIGS. 7A-7C), skin (Langerhans cells), kidney, heart, and peritoneum. Additional analysis of monocyte populations in the brain reveals a loss of the CD45low CD11b+ CNS cell population, traditionally recognized to be microglia, while the CD45hi CD11b+ population, traditionally recognized to be macrophages, remains stable (see FIG. 7D-7E). No other macrophages or monocytes are impacted, but monocytes and their progenitors in bone marrow lack surface CSF1R.

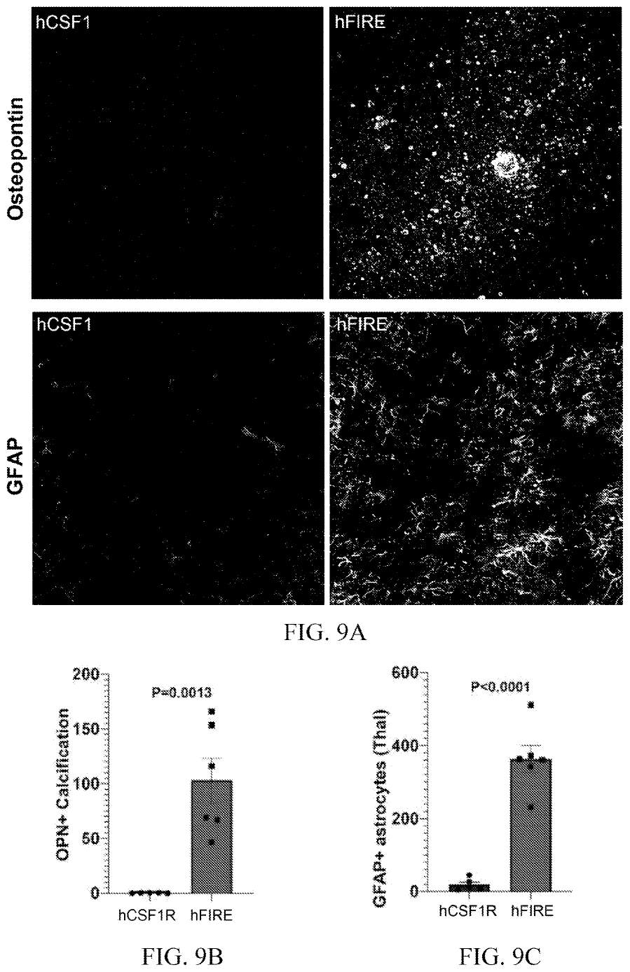

By 6 months of age, Csf1rΔFIRE/ΔFIRE mice exhibit the ALSP pathology of axonal spheroids (see FIGS. 8A-8B), focal swellings within neurons that contain insoluble neurofilament and amyloid precursor proteins. Additionally, brain calcification and reactive astrogliosis (see FIGS. 9A-9C) are hallmarks of the Csf1rΔFIRE/ΔFIRE mouse pathology and have been detected in ALSP patients.

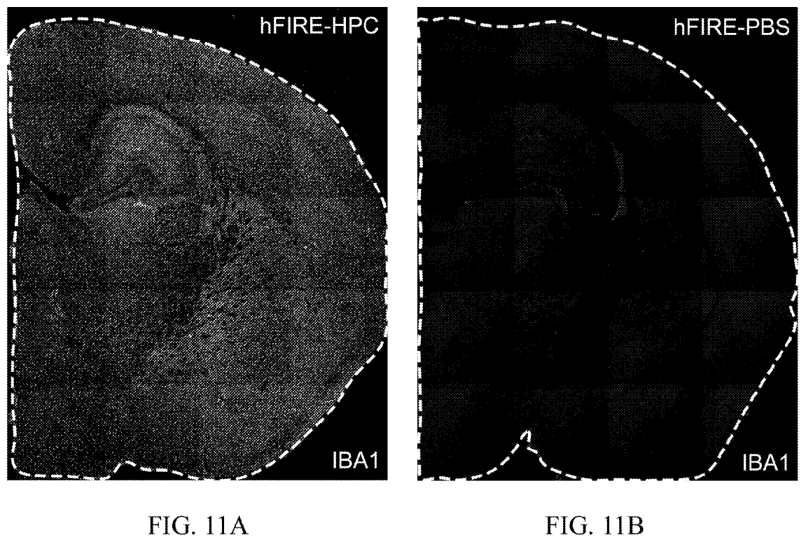

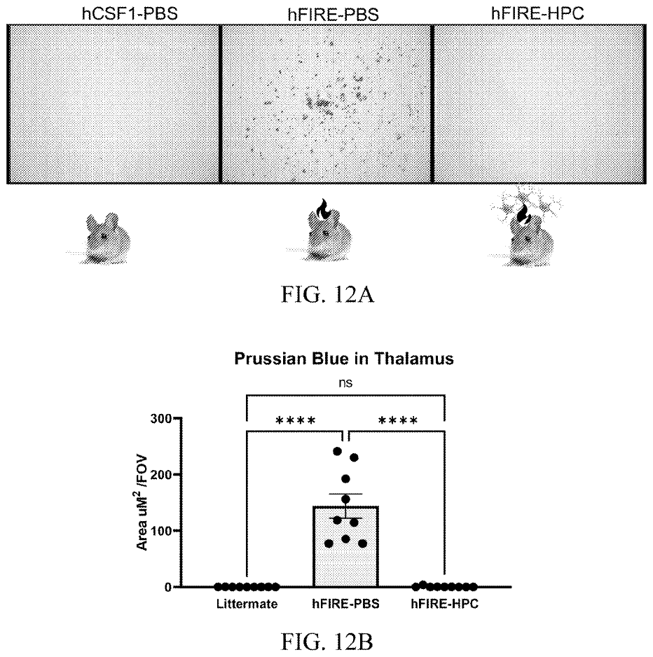

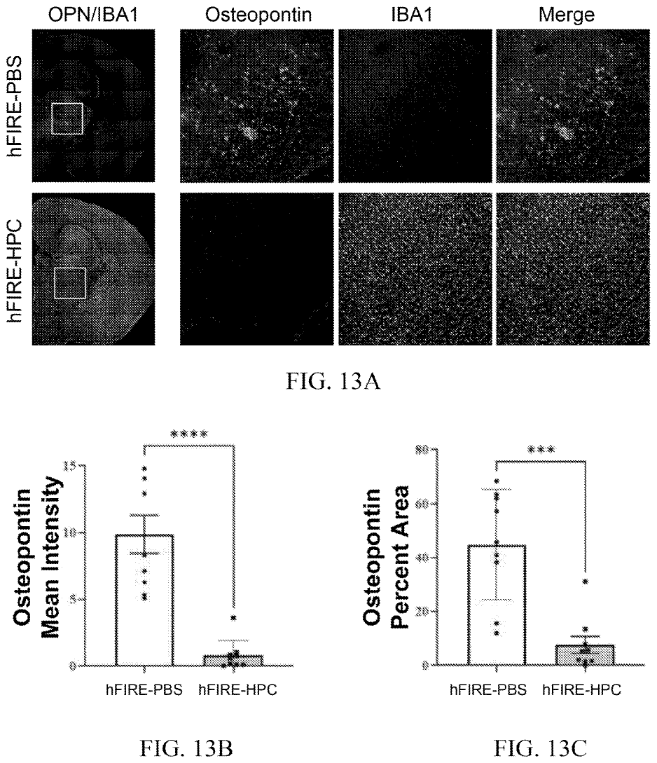

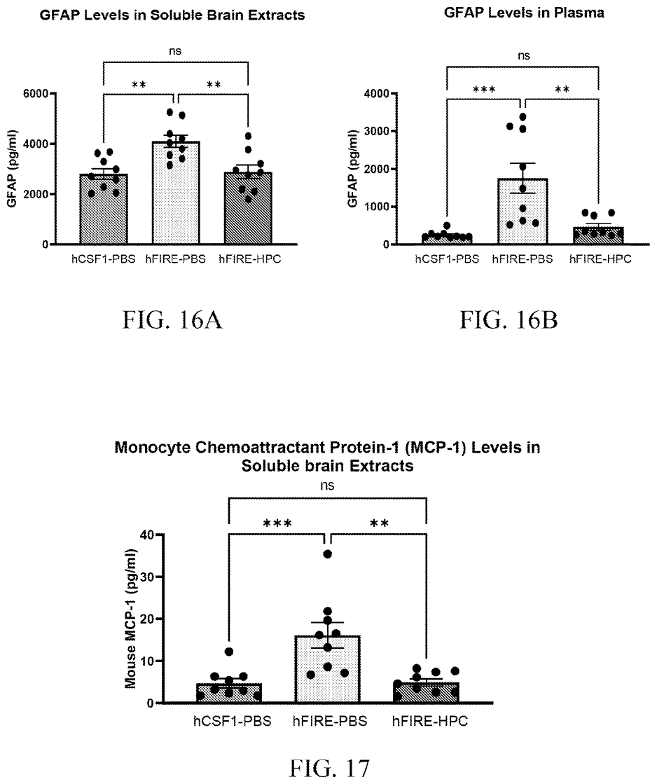

When assessed at 8.5 months of age by immunohistological analysis, the Csf1rΔFIRE/ΔFIRE mice exhibited an abundance of microbleeds in the thalamus and throughout the brain (FIGS. 11A-11B), a nearly complete loss of Iba1-immunoreactive microglia throughout the brain (FIG. 12A), brain calcification (FIGS. 13A-13B), Unlike brain sections obtained from hCSF1R wildtype littermates (hCSF1R-WT), axonal spheroids (as described by Sundal et al. J Neurol Sci. 2012 Mar. 15; 314(1-2):130-7) were abundant in the thalamus, hippocampus, white matter, cortex, fornix, and throughout the brain of Csf1rΔFIRE/ΔFIRE mice aged 8.5 months, which is indicated by the presence of SM1312 antibody intensity at complex and dense axonal bundles (FIGS. 16A-16B) as well as high-intensity expression of the lysosomal marker LAMP1 (FIGS. 17A-17D).

### Example 5: Transplantation of Human iPSC-Derived Microglia into Csf1ΔFIRE/ΔFIRE Mice

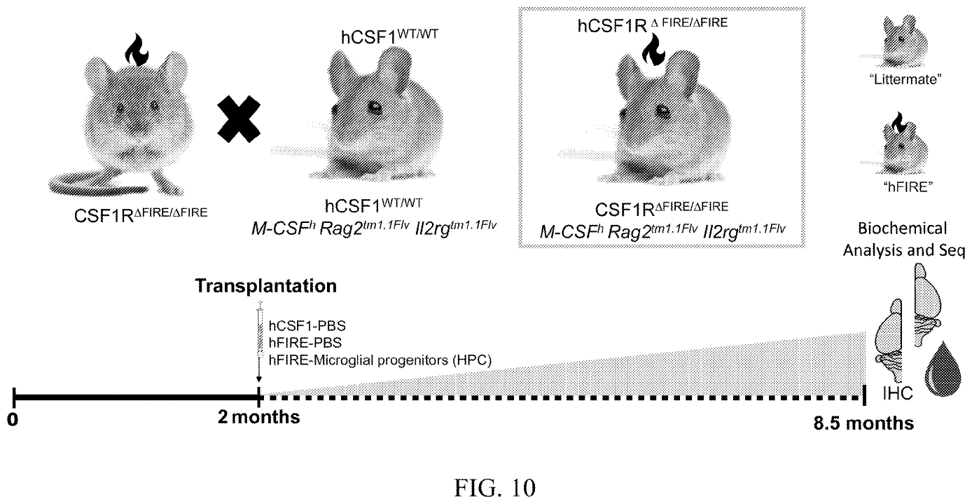

There are several pathological hallmarks of ALSP, including depletion of microglia, axonal spheroids, astrogliosis and calcification deposits. These hallmarks are recapitulated in the Csf1rΔFIRE/ΔFIRE mouse. The Csf1rΔFIRE/ΔFIRE mouse represents a severe instance of CSF1R dysfunction. The therapeutic potential of the human iMGLs described herein was assessed after interbreeding Csf1rΔFIRE/ΔFIRE with M-CSF^{h} Rag2^{tm1.1Flv} I12rg^{tm1.1Flv} mice to facilitate inter-special transplantation of the human iMGLs (FIG. 10). The M-CSFh Rag2t^{h} Rag2^{tm1.1Flv} I12rg^{tm1.1Flv} mice express a humanized CSF1, which is the ligand of CSF1R. Expression of the human CSF1 is required for the survival of transplanted human iMGLs in the mouse brain. The CSF1 endogenously expressed in the hCSF1R-WT mouse (i.e., mouse CSF1) cannot signal through the human CSF1R receptor, and the knockout of Rag2^{tm1.1Flv} and I12rg^{tm1.1Flv} genes makes the immune system of the mouse model accept the transplanted human iMGLs and avoids graft rejection.

Two-month-old Csf1rΔFIRE/ΔFIRE mice were targeted for transplantation of genetically unmodified human iPSC-derived HPCs (microglial progenitors) or iMGLs expressing functional hCSF1R to determine whether they were capable of engrafting in the brain and could prevent or reverse ALSP pathological phenotypes since patients are typically first diagnosed with ALSP in adulthood from age 20-40. For these experiments, 2-month-old Csf1rΔFIRE/ΔFIRE mice were intracranially injected with either phosphate-buffered saline solution (hFIRE-PBS) or 5×10⁵ human iPSC-derived HPCs (hFIRE-HPCs). Additionally, hCSF1R-WT littermates received an intracranial injection of PBS (control hCSF1-PBS) as the control group. All animals were then sacrificed at 8.5 months of age and subjected to immunohistochemical and biochemical analyses to assess whether transplantation of human iPSC-derived HPCs or iMGLs expressing CSF1R could prevent or rescue ALSP pathological phenotypes.

Within 8 weeks following transplantation, human iPSC-derived HPCs completely engrafted throughout the brain of hFIRE-HPC mice, demonstrated by the vast distribution of IBA1 expression in brain sections containing the hippocampus, midbrain, striatum, thalamus, and cerebral cortex. (FIG. 11A). In contrast, hFIRE-PBS mice exhibited sparse and faint IBA1 expression throughout the brain, which was primarily localized to the periventricular area and indicates that the Csf1rΔFIRE/ΔFIRE mutation does not affect meningeal cells (FIG. 11B). No IBA1 expression was detected in the hippocampus, cerebral cortex, thalamus, striatum, or midbrain of hFIRE-PBS mice.

Importantly, transplantation of genetically unmodified human iPSC-derived HPCs or iMGLs caused the complete rescue of several ALSP-related pathological phenotypes in the Csf1rΔFIRE/ΔFIRE mice. Brain sections prepared from 8.5-month-old hFIRE-HPC mice did not exhibit microbleeds, as evidenced by the near-absent Prussian blue staining intensity which was also comparable to hCSF1-PBS controls (FIGS. 12A-12B). In contrast, brain tissue of hFIRE-PBS mice displayed extensive Prussian blue staining in comparison to both control hCSF1-PBS and hFIRE-HPC groups (FIGS. 12A-12B). These data indicate that while absence of microglia causes microbleeds, transplantation of human iPSC-derived HPCs or iMGLs can completely rescue this ALSP-related phenotype.

Additionally, brain calcification was imaged and measured by examining the accumulation of the calcium binding protein Osteopontin (OPN) in Csf1rΔFIRE/ΔFIRE mice. Vehicle (PBS) or human iPSC-derived microglia (IBA-1) progenitors were transplanted via intraparenchymal injection into 2-month-old Csf1rΔFIRE/ΔFIRE mice and brains were examined 6 months later. Confocal stitches of half brains revealed a large amount of OPN immunoreactivity within the thalamus and basal ganglia and an absence of microglia in PBS-injected ALSP mice. In contrast, transplantation of 500,000 human IPSC-HPCs led to complete engraftment of the microglia niche and a corresponding reduction of OPN. Blinded quantification of confocal Z-stack images demonstrated a highly significant and near-complete reduction in OPN mean intensity and percent area (see FIGS. 13A-13C). Accordingly, it was observed that the transplantation of human iPSC-derived microglia via intraparenchymal injection into 2-month-old mice resulted in the clearance of protein deposits associated with calcification of blood vessels that is sustained for at least 6 months. Representative photomicrographs are shown in FIG. 13A.

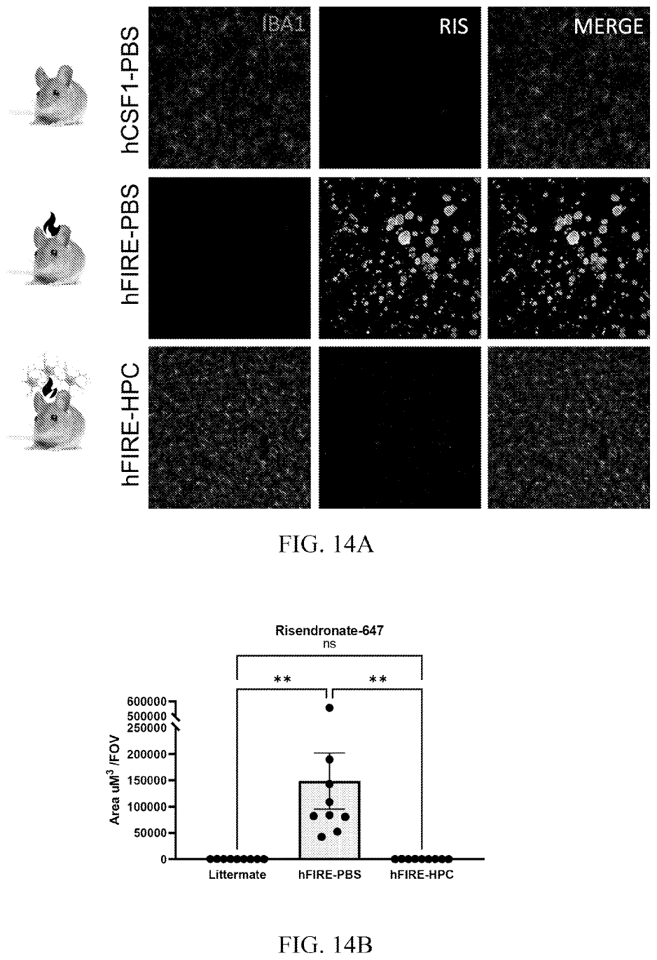

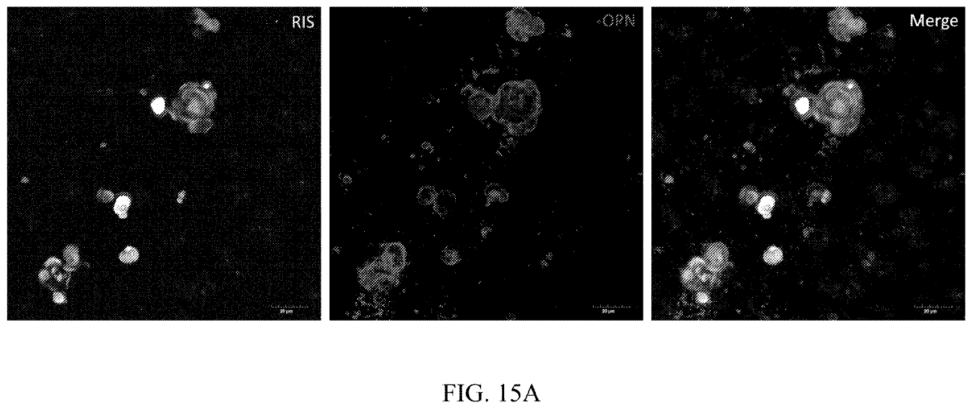

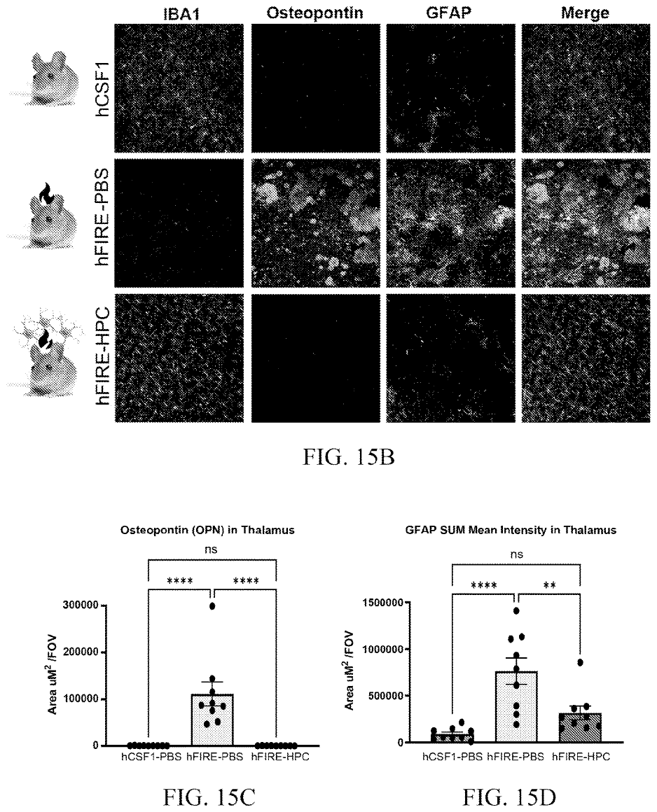

The transplantation of human iPSC-derived HPCs also completely rescued calcification of blood vessels in the brains of Csf1rΔFIRE/ΔFIRE mice to CSF1R-PBS levels. Brain sections prepared from hFIRE-HPC mice exhibited robust engraftment of Iba1+ iMGLs but did not display any calcium deposits, as indicated by the absence of Risendronate-647 (RIS-647) signal intensity (FIGS. 14A-14B). However, brain sections obtained from hFIRE-PBS mice had a high prevalence RIS-647-stained hydroxyapatite calcium deposits and a complete lack of Iba1 expression in comparison to both hFIRE-HPC and CSF1R-PBS mice (FIG. 14B). Human iPSC-derived HPC transplantation also completely rescued ALSP-associated accumulation of secreted proinflammatory cytokines such as OPN, which had abundant immunoreactivity in the thalamus of hFIRE-PBS mice and colocalized with RIS-647-stained calcium deposits (FIG. 15A-15C). In contrast, OPN accumulation was essentially absent in thalamic brain sections of hFIRE-HPC and CSF1R-PBS mice (FIG. 15B-15C).

Astrogliosis in the brains of Csf1rΔFIRE/ΔFIRE mice was also robustly reduced within 6 months following transplantation of human iPSC-derived HPCs. The cumulative intensity of GFAP immunoreactivity in brain sections of hFIRE-HPC and CSF1R-PBS mice was comparable, while hFIRE-PBS brain tissue exhibited GFAP immunoreactivity that was significantly increased compared to both hFIRE-HPC and control hCSF1-PBS groups by at least 2-fold (FIG. 15D). Transplantation of human iPSC-derived HPCs also reduced GFAP levels in soluble brain extracts (FIG. 16A) and blood plasma (FIG. 16B) in hFIRE-HPC mice to levels comparable to CSF1R-PBS controls, while GFAP levels in soluble brain extracts and blood plasma were significantly increased in hFIRE-PBS mice compared to both hFIRE-HPC and CSF1R-PBS groups. These data demonstrated that human iPSC-derived HPC transplantation can engraft into microglial niches and prevent or rescue astrogliosis in a microglia-depleted central nervous system. Additionally, these data suggest that the ability to detect changes in blood plasma GFAP levels to distinguish the ALSP disease state from both normal and treated conditions would make GFAP a promising candidate biomarker to determine hiPSC-HPC transplant efficiency.

Immunological mechanisms can engage migration of peripheral immune cells including T cells and macrophages into the brain in pro-inflammatory conditions. The protein Monocyte Chemoattractant Protein-1 (MCP-1 or CCL2) is a pro-inflammatory chemokine and chemoattractant that facilitates this process. After 6.5 month following hiPSC-HPC transplantation, MCP-1 levels in soluble brain extracts were rescued in hFIRE-HPC mice and reduced to levels comparable to hCSF1-PBS control samples, while MCP-1 levels in hFIRE-PBS samples were increased 3-fold compared to control hCSF1-PBS and hFIRE-HPC samples (FIG. 17). These data suggest that engraftment of hiPSC-HPCs can prevent or reverse the homeostatic recruitment of peripheral immune cells into a microglia-depleted brain.

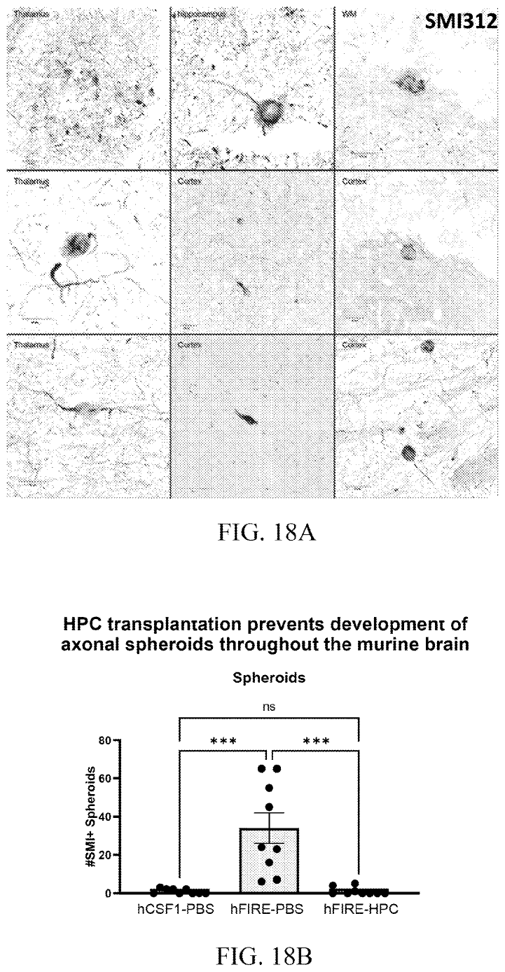

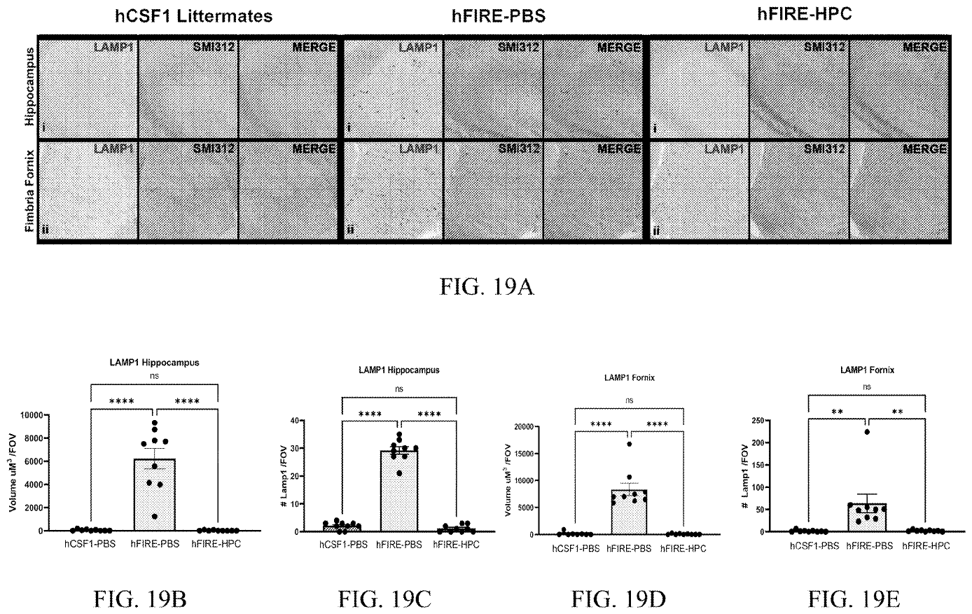

The development of ALSP disease-associated axonal spheroids was completely rescued in hFIRE-HPC mice after 6.5 months following hiPSC-HPC transplantation. This is demonstrated by the minimal number of neurofilament SMI312-immunoreactive spheroids present throughout the brains of hFIRE-HPC. The number of SMI312+ spheroids in hFIRE-HPC brain sections containing the hippocampus, cortex, thalamus, and white matter (WM) was comparable to hCSF1-PBS control brain sections and reduced by ˜90% compared to brain sections obtained from hFIRE-PBS mice (FIGS. 18A-18B). Similarly, transplantation of hiPSC-HPCs also completely rescued and nearly eliminated LAMP1-immunoreactive axonal spheroids in brain sections obtained from hFIRE-HPC mice containing the hippocampus (FIGS. 19A-19C) and fornix (FIGS. 19A, FIG. 19D-19E) to levels indistinguishable from control hCSF1-PBS brain sections and reduced by >90% compared to hFIRE-PBS brain sections.

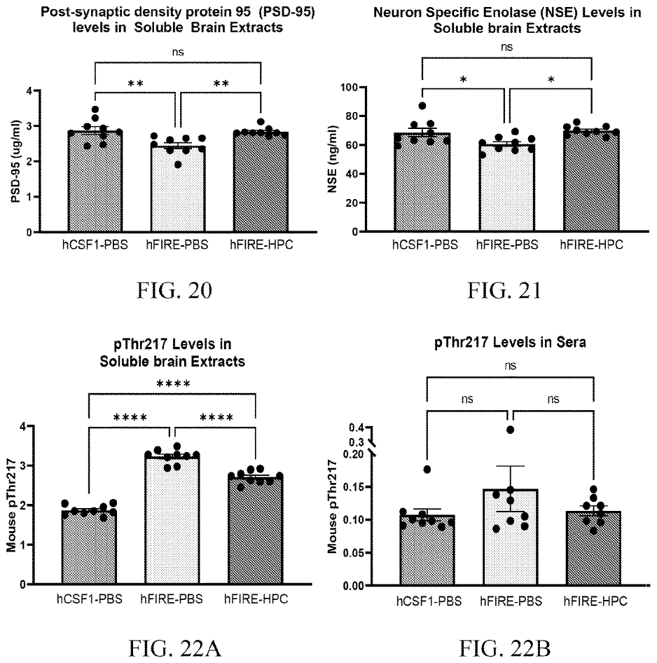

Compared to age-matched control hCSF1-PBS littermates, hFIRE-PBS mice aged 8.5 months also exhibited reduced expression of PSD95, which is a neuronal postsynaptic marker of excitatory synapses in the central nervous system, in soluble brain extracts (FIG. 20). At the very least, these data demonstrate that loss of microglia also affects neuron-specific mechanisms, likely including synaptic structure and/or abundance. In contrast, transplantation of hiPSC-HPCs rescued PSD95 in hFIRE-HPC brain extracts to levels comparable to those of control hCSF1-PBS (FIG. 20).

Microglia function is critical for homeostasis of neurons throughout the lifespan of an organism, and thus Csf1rΔFIRE/ΔFIRE mice were assessed for alterations in the expression levels of neuron-specific enolase (NSE), which is a neuron-specific cell marker. Similar to the effects of CSF1R deletion on PSD95 expression, the hFIRE-PBS mice also exhibited reduced NSE levels measured in soluble brain extracts (FIG. 21), indicating that loss of CSF1R is required for maintenance of neuronal homeostasis by potentially promoting neuronal survival or development. However, the reduced NSE levels were rescued by transplantation of hiPSC-HPCs into the hFIRE-HPC mice, as shown by the comparable NSE levels of hFIRE-HPC and control hCSF1-PBS mice (FIG. 21). Taken together, these data not only suggest that loss of microglia may potentially cause synapse loss and neuronal cell death, which are hallmark aspects of neurodegeneration, but also demonstrate that transplantation and repopulation of the brain with iMGLs has potential to ameliorate or delay progression of these pathological phenotypes.

Supporting this interpretation, the same soluble brain extracts obtained from control hCSF1-PBS mice displayed increased levels of phosphorylated Tau protein at threonine-217 (Tau-pT217), while Tau-pT217 levels in soluble brain extracts of hFIRE-HPC mice were significantly reduced compared to hFIRE-PBS samples (FIG. 22A). Tau phosphorylation is associated with neurodegenerative disorders such as Alzheimer's Disease, and thus, it is possible that the hyperphosphorylation of Tau-pT217 that was observed in control hCSF1-PBS mice may indicate that a loss of microglia can also result in neurodegeneration.

Furthermore, a statistical trend indicated a likely increase of Tau-pT217 levels in plasma serum of hFIRE-PBS mice compared to hCSF1-PBS littermate controls, and no changes in plasma Tau-pT217 levels of were observed between hFIRE-HPC and control hCSF1-PBS animals (FIG. 22B). Although serum Tau-pT217 levels were not significantly altered by CSF1R expression or microglial engraftment (FIG. 22B), there exists a trend toward a similar pattern of increased Tau-pT217 levels in hFIRE-PBS mice and decreased Tau-pT217 levels in hFIRE-HPC mice.

### Example 6: Transplantation of Human iPSC-Derived HPCs Restores the Microglial Transcriptomic Signature in Csf1rΔFIRE/ΔFIRE Mice

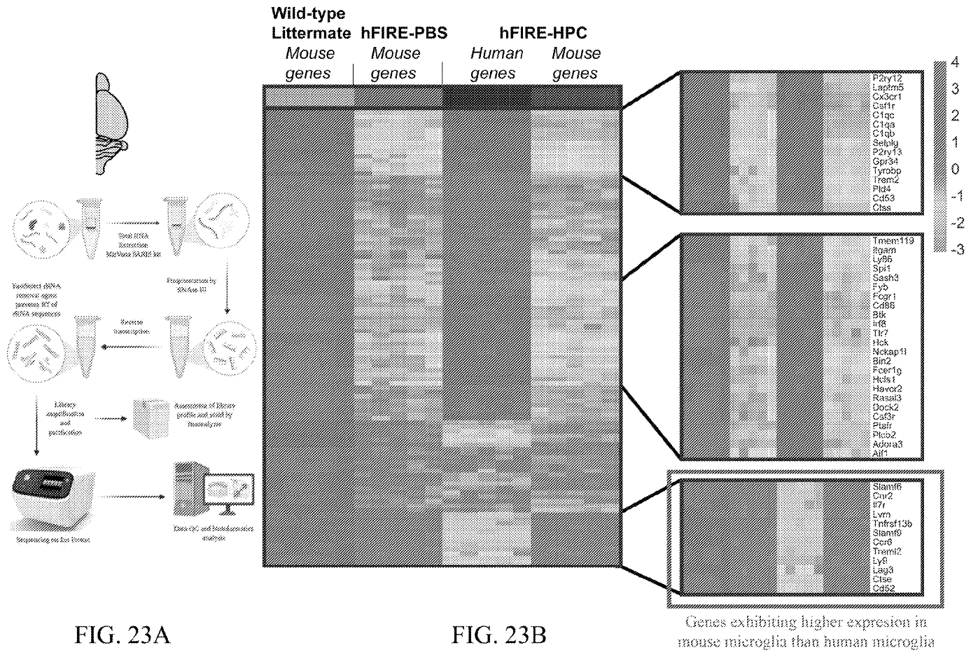

Two-month-old Csf1rΔFIRE/ΔFIRE mice were intracranially injected with either phosphate-buffered saline solution (hFIRE-PBS) or about 5×10⁵ human iPSC-derived HPCs (hFIRE-HPCs). Additionally, hCSF1R-WT littermates received an intracranial injection of PBS (control hCSF1-PBS) and served as the control group. All animals were then sacrificed at 8.5 months of age, and then their brains were extracted and processed for bulk RNA sequencing. Briefly, as shown in FIG. 23A, brains were rapidly extracted from the skull of each animal, and total RNA was extracted from the tissue with the mirVana™ PARIS™ RNA and Native Protein Purification kit (Cat #: A M1556, ThermoFisher Scientific), and QIAseq FastSelect agent was added to the total RNA sample to prevent reverse transcription of ribosomal RNA transcripts. Isolated total RNA was then heat-fragmented with a RNAse III digestion for 5 minutes at 37° C. in order to achieve mRNA fragments of an average length of approximately 200 nucleotides. Reverse transcription polymerase chain reaction (RT-PCR) was then performed to create a cDNA library of the mRNA fragments destined for RNA sequencing on the Ion Proton™ sequencer. Assessment and yield of the cDNA library was analyzed with the Agilent 2100 Bioanalyzer prior to sequencing. Bioinformatic analysis and quality control assessments were finally performed after sequencing was completed. The experiment described in FIG. 23B includes control hCSFT-PBS (n=5), hFIRE-PBS (n=5), and hFIRE-HPC (n=5).

Transplantation of human iPSC-derived HPCs or iMGLs expressing CSF1R restored the transcriptomic landscape and mRNA levels of microglia-related genes to the brains of hFIRE-HPC mice (FIG. 23B). The bulk RNA sequencing data revealed differential expression of microglia genes that were significantly downregulated (LFC<−3; FDR<0.01) in hFIRE-PBS samples compared to control hCSF1-PBS samples, including many canonical microglial genes such as: P2ry12, Laptm5, Cx3cr1, Csf1r, C1qc, C1qa, C1qb, Selp1g, P2ry13, Gpr34, Tyrobp, Trem2, Pld4, Cd53, Ctss. Additionally, comparison of hFIRE-HPC samples identified that transplantation of human HPCs or iMGLs does not significantly increase (LFC<−1; FDR<0.05) background expression within the subset of microglia-related genes including: P2ry12, Cx3cr1, Csf1r, C1qc, C1qa, C1qb, Selp1g, P2ry13, Gpr34, Tyrobp, Trem2, Pld4, Cd53, Ctss. In contrast, examination of human microglial reads from the hFIRE-HPC samples (hFIRE_HPC_Microglia) identified expression levels of canonical microglia-related genes that were comparable to the analogous mouse microglia-related genes present in the control hCSF1-PBS samples, including: P2ry12, Laptm5, Cx3cr1, Csf1r, C1qc, C1qa, C1qb, Selp1g, P2ry13, Gpr34, Tyrobp, Trem2, Pld4, Cd53, Ctss.

A second group of microglia-related genes exhibited lower average expression levels while still being differentially expressed between hFIRE-PBS and control hCSF1-PBS samples (LFC<−2; FDR<0.01), including: Tmem119, Itgam, Ly86, Spi1, Sash3, Fyb, Fcgr1, Cd86, Btk, Trfg, Tlr7, Hck, Nckapl L, Bin2, Fcer1g, Hcls1, Rasa13, Dock2, Csf3r, Ptafr, Plcb2, Adora3, and Aif1. Additionally, comparison of hFIRE-HPC_Brain samples to hCSF1-PBS samples identified that transplantation of HPCs or iMGL does not significantly (LFC>+−1; FDR<0.05) alter background expression levels within the subset of microglia-related genes including: Tmem119, Itgam, Ly86, Spi1, Sash3, Fyb, Fcgr1, Cd86, Btk, IrfS, Tlr7, Hck, Nckap1L, Bin2, Fcer1g, Hcls1, Rasal3, Dock2, Csf3r, Ptafr, Plcb2, Adora3, and Aif1. Of note, expression levels of the following genes were comparable between hFIRE-HPC and control hCSF1-PBS brain samples, including: Tmem119, Itgam, Ly86, Spi1, Sash3, Fyb, Fcgr1, Cd86, Btk, Irf8, Tlr7, Hck, Nckap1L, Bin2, Fcer1g, Hcls1, Rasa13, Dock2, Csf3r, Ptafr, Plcb2, Adora3, and Aif1.

A third group of microglia-related genes is noted because human reads within hFIRE-HPC_Microglia samples exhibited lower expression compared to hSCF1-PBS, hFIRE-PBS, and hFIRE-HPC_Brain samples, including: Slamf6, Cnr2, 117r, Lvrn, Tnfrsfl3b, SlamT9, Ccr6, Trem12, Ly9, Lag3, Ctse, and Cd52. However, of these genes, the following genes are found to be more highly expressed in mouse microglia than in human microglia (brainrnaseq.org): Slamf6, Cnr2, 117r, Tnfrsfl3b, Slamf9, Ccr6, Trem12, Ly9, Lag3, Ctse, and Cd52. This group highlights species-specific gene expression profiles, which also provides an indication of high sensitivity of the experimental methods utilized and the sound quality of the RNA sample and cDNA library preparation.

Taken together, transplantation of CRISPR-engineered human iPSC-derived HPCs with intact CSF1R expression are capable of engrafting throughout a microglia-depleted brain in a mouse model of ALSP and restore the expression levels of microglia-associated gene networks to levels comparable to that of endogenous wildtype levels.

Taken together, transplantation of CRISPR-engineered human iPSC-derived HPCs with intact CSF1R expression are capable of engrafting throughout a microglia-depleted brain in a mouse model of ALSP and restore the expression levels of microglia-associated gene networks to levels comparable to that of endogenous wildtype levels.

### Example 7: Transplanted HPCs and iMGLs Derived from a hiPSC Line of a Healthy Donor Efficiently Engraft Throughout the Brain of Csf1rΔFIRE/ΔFIRE Mice and Prevent ALSP-Related Phenotypes

According to the methods previously described, an hiPSC line from a healthy human donor (A75 or A77) was differentiated in vitro for either one day (D1) or 28 days (D28) in differentiation media to generate microglial progenitors (A75, A76, or A77 HPCs) and microglial-like cells (A75, A76, or A77 iMGLs), respectively.

At 2 months of age, Csf1rΔFIRE/ΔFIRE were intracranially injected with about 5×10⁵ of either D1 A75 HPCs (hFIRE-A75 D1) or D28 A75 iMGLs (hFIRE-A75 D28) and then assessed at 5.5 months of age by immunohistological and biochemical analyses for correction of ALSP-related phenotypes in comparison to Csf1rΔFIRE/ΔFIRE mice which received an intracranial injection of PBS (hFIRE-PBS).

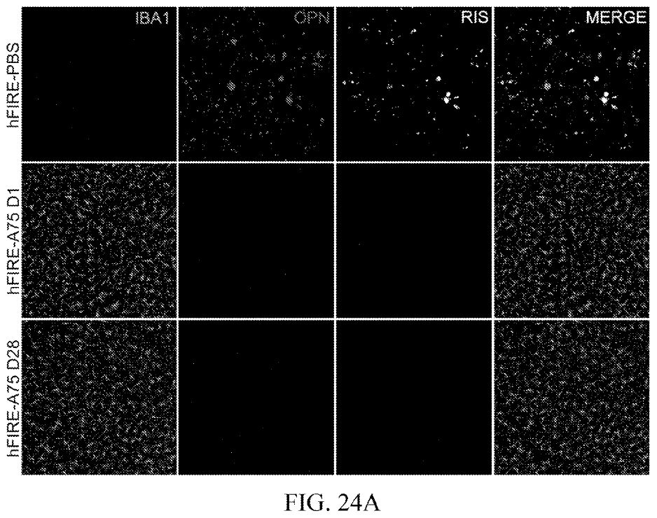

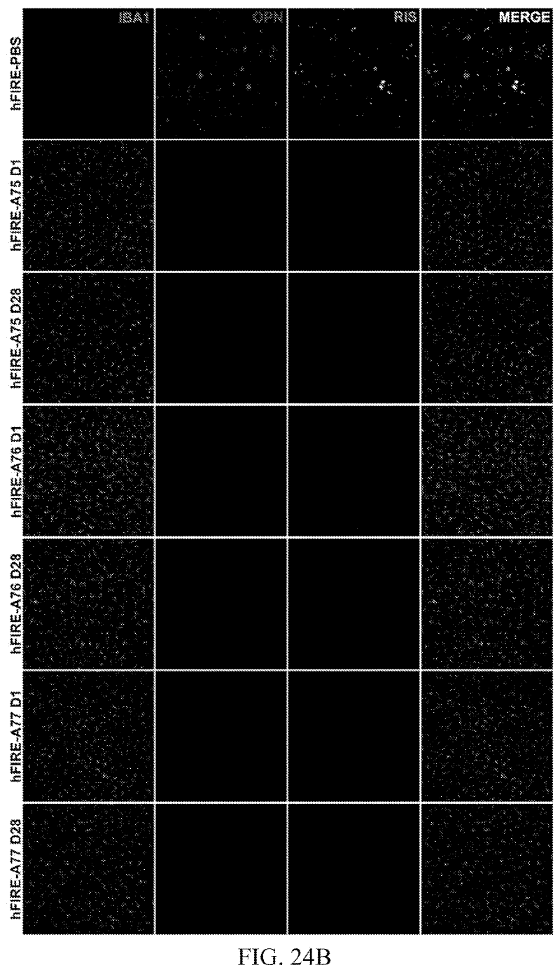

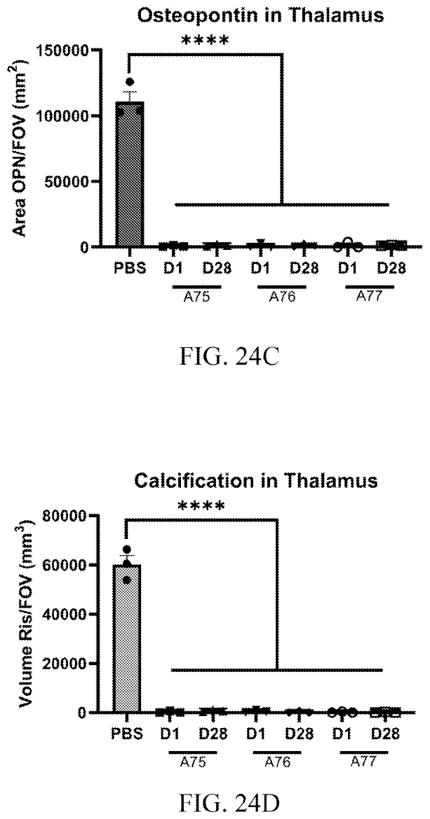

Both hFIRE-A75 D1 (HPCs) and hFIRE-A75 D28 (iMGLs) had robustly engrafted throughout the thalamus of Csf1rΔFIRE/ΔFIRE mice, which is indicated by the high density of Iba1-immunoreactive cells in the brain sections shown in FIG. 24A. In contrast, thalamic sections of hFIRE-PBS mice essentially lacked Iba1 immunoreactivity which confirms the absence of microglia (FIG. 24A).

As expected, transplantation of either genetically unmodified hFIRE-A75 D1 (HPCs) or hFIRE-A75 D28 (iMGLs) caused a complete rescue and reversal of the ALSP-related accumulation of the pro-inflammatory cytokine OPN and eliminated RIS-647-immunoreactive calcium deposits, whereas hFIRE-PBS brain tissue exhibited abundant OPN accumulation and RIS-647+ calcium crystals (FIG. 24A). The same experiment was repeated in order to transplant 2-month-old Csf1rΔFIRE/ΔFIRE mice with either HPCs or iMGLs from A75, A76, or A77 (6 total transplanted healthy donor populations) and assess for correction of ALSP-related phenotypes by immunohistological and biochemical analyses when the mice were 8.5 months of age. These data demonstrated that transplantation of “wildtype” HPCs derived from A75, A76, or A77 iPSC lines were equally capable of effective and dense engraftment throughout the microglia-depleted brain of Csf1rΔFIRE/ΔFIRE mice, as indicated by the high density of cells with Iba1 immunoreactivity for all transplanted A75, A76, and A77-derived HPCs and iMGLs populations (FIG. 24B). Furthermore, all transplanted A75, A76, and A77-derived HPCs and iMGLs populations exhibited a complete reversal of OPN accumulation and formation of RIS-647+ calcium deposits in brain sections of hFIRE mice (FIGS. 24C-24D). As expected, Iba1 immunoreactivity was essentially absent in thalamic brain sections of hFIRE-PBS mice, which also displayed elevated OPN accumulation and RIS-647+ calcification (FIGS. 24C-24D).

Together, these data demonstrate that hiPSC-derived HPCs and mature iMGLs with intact hCSF1R expression are equally capable of engrafting and migrating throughout the brain to populate a microglial niche, which may consequently reverse aspects of the ALSP phenotype and restore homeostasis in a microglia-depleted brain.

### Example 8: CRISPR-Corrected Point Mutations in ALSP Patient iPSC Lines Rescue ALSP Disease-Associated Deficits in Microglial Proliferation

As described above, the genetic correction of single SNPs within hCSF1R was accomplished by CRISPR-guided insertion of ssODNs containing the corrected wildtype human CSF1R nucleic acids in 6 individual ALSP patient hiPSC lines heterozygous for point mutations in hCSF1R (see TABLE 9, FIG. 4, FIG. 5). The ssODNs and sgRNAs of the CRISPR-Cas9 RNP complex used for correcting each ALSP patient iPSC line are shown in TABLE 10 and TABLE 11, respectively.

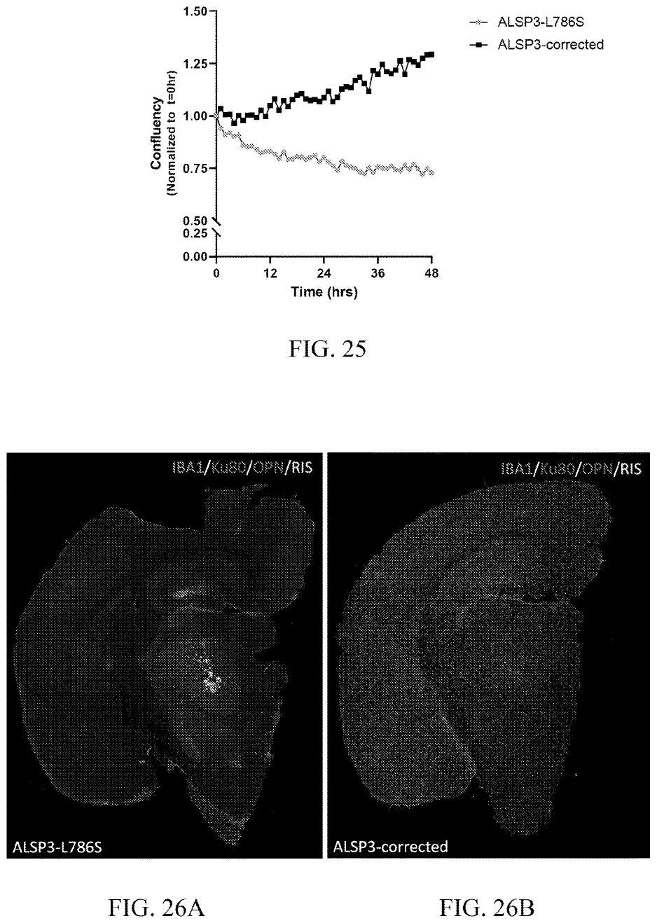

After successful correction of hCSF1R, the 6 hCSF1R-corrected ALSP patient hiPSC lines were separately differentiated for 28 days in vitro to generate iMGLs from each ALSP patient iPSC line, and the proliferation ability of each corrected iMGL population was assessed (e.g., confluency, marker expression, etc.) and compared to the individual ALSP patient hiPSC lines harboring the point mutation. Over 48 hours, the confluency of ALSP3-L786S iMGLs decreased over time to about 75% of the confluency measured at time=0 hours, which indicated that either the ALSP3-L786S iMGLs did not proliferate or had died at a rate faster than they proliferated (FIG. 25). In contrast, the CRISPR-edited ALSP3-corrected iMGLs exhibited steady and continual increases in confluency of the culture dish over the 48-hour period without plateauing, which suggested that the viability and/or proliferation rates of ALSP3-corrected iMGLs have been restored or at least markedly improved (FIG. 25).

### Example 9: CRISPR-Corrected Point Mutations in ALSP Patient iPSC Line Rescues Disease-Associated Deficits in Microglial Proliferation

Once the hCSF1R-corrected ALSP patient hiPSC lines were successfully generated and differentiated into hiPSC-HPCs (see TABLES 9-11, FIG. 4, FIG. 5), each ALSP-corrected hiPSC-HPC population (about 2.5×10⁵ cells administered per animal) was individually administered via targeted bilateral intracranial injection into the hippocampus of Csf1rΔFIRE/ΔFIRE mice at 2-2.5 months of age. Additionally, another group of age-matched Csf1rΔFIRE/ΔFIRE mice and hCSF1^{WT/WT} mice received intracranial injections of PBS (hFIRE-PBS; control hCSF1-PBS). Animals were then sacrificed 6 weeks after transplantation, and then iMGL engraftment and ALSP disease-related phenotypes were assessed by immunohistochemistry, RNA sequencing, and additional biochemical methods.

For example, ALSP3-L786S iMGLs (which are hCSF1R-haploinsufficient) inefficiently engrafted into the brains of Csf1rΔFIRE/ΔFIRE mice, as demonstrated by the localized immunoreactivity of Iba1 and Ku80 (human-specific nuclear cell marker) surrounding the injection site and largely confined within the hippocampus (FIG. 26A). At best, some engraftment of ALSP3-L786S engraftment occurred at near-undetectable levels within the dorsal basal ganglia. However, Iba11+ and Ku80+ ALSP3-corrected iMGLs robustly engrafted throughout the microglia-depleted brains of Csf1rΔFIRE/ΔFIRE mice and robustly proliferated and migrated to repopulate the microglial niche after only 6 weeks following transplantation (FIG. 26B).

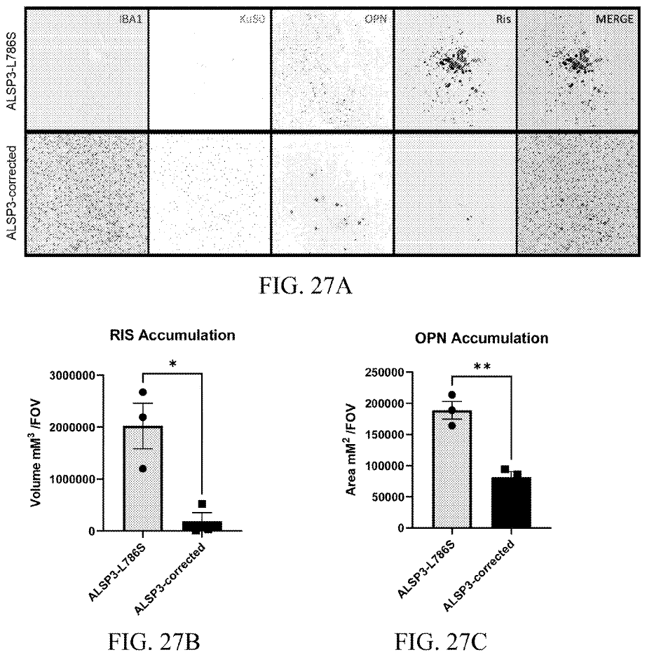

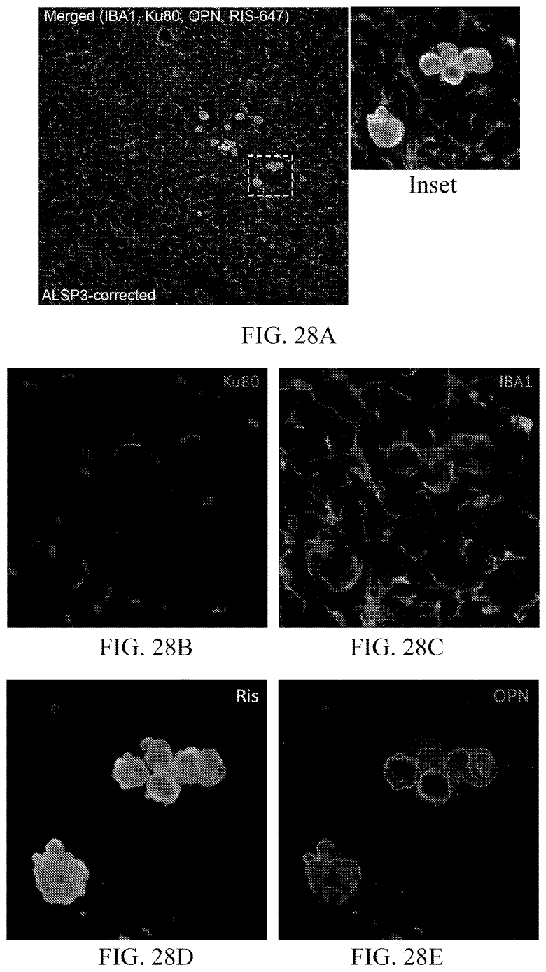

Transplantation of patient-derived ALSP3-L786S HPCs also failed to prevent or reverse brain calcification detected by RIS-647-immunoreactivity (RIS) and osteopontin accumulation (OPN) within the thalamus of hFIRE-HPC mice (FIG. 26A, FIGS. 27A-27C), while levels of both RIS-647+ brain calcification and OPN accumulation were nearly eliminated 6 weeks after transplantation of ALSP3-corrected-HPCs (FIGS. 26B, FIG. 27A-27C). ALSP3-corrected iMGLs also demonstrated the ability to surround RIS-647+ calcium crystals and secreted OPN (FIGS. 28A-28E), which indicates that ALSP-corrected iMGLs have adopted innate and adaptive immune responses, are immunosurveillant, and capable of surrounding and likely phagocytosing pro-inflammatory stimuli and byproducts of ALSP pathology.

Taken together, these data demonstrate that the CRISPR-mediated correction of hCSF1R expression in ALSP patient hiPSCs coupled with the differentiation and transplantation methods described herein is a powerful therapeutic strategy for the treatment of microglial dysfunction, microglial proliferation deficits, and neurological diseases affecting microglial function such as ALSP, Alzheimer's Disease, Huntington Disease, and Parkinson's Disease.

### Example 10: Csf1rΔFIRE/ΔFIRE Mice Exhibit Pathological ALSP Phenotypes Early in Development

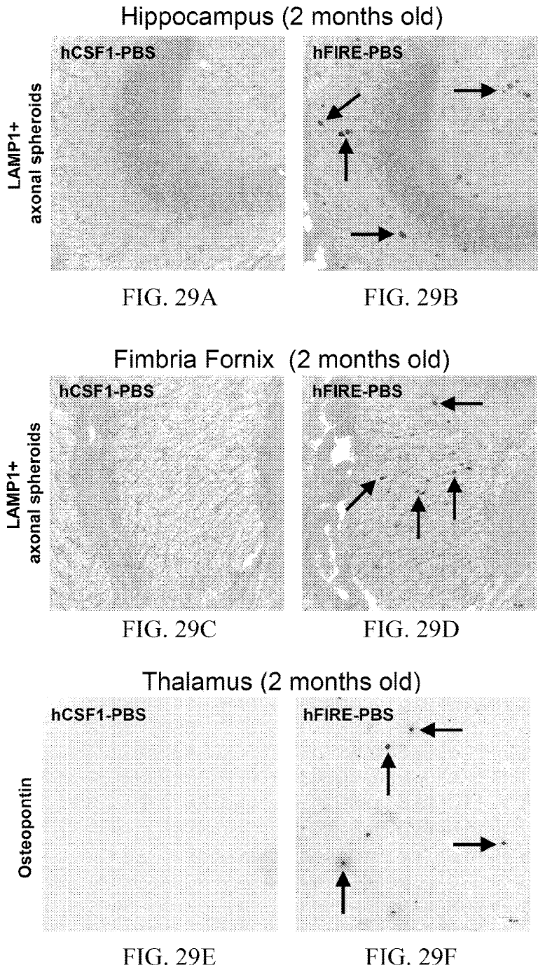

Additional experiments demonstrated that Csf1rΔFIRE/ΔFIRE mice exhibit signs of ALSP pathologies such as axonal spheroids and accumulation of pro-inflammatory cytokines as early as 2 months of age (FIG. 29A-29F), indicating that the CRISPR-mediated insertion and repair strategies of the disclosure can be used as a strategy for reversing the ALSP pathology in cases where therapeutic intervention occurs later in development. The prevalence of LAMP1-immunoreactive axonal spheroids was assessed in the hippocampus, fornix, fimbria, and thalamus of brain sections obtained from 2-month-old Csf1rΔFIRE/ΔFIRE mice and age-matched hCSF1R^{WT/WT} littermate controls. Although hippocampal sections of hCSF1R^{WT/WT} control littermates were completely devoid of LAMP1+ axonal spheroids (FIG. 29A), hippocampal sections of Csf1rΔFIRE/ΔFIRE mice exhibited an abundance of dense LAMP1+ puncta, indicating that ALSP-associated development of axonal spheroids is present in Csf1rΔFIRE/ΔFIRE as early as 2 months of age. (FIG. 29B) Similarly, LAMP1+ axonal spheroids were lacking in the fornix and fimbria of brain sections obtained from hCSF1R^{WT/WT} (FIG. 29C) but were abundant in the fornix and fimbria of brain sections obtained from Csf1rΔFIRE/ΔFIRE mice (FIG. 29D), which indicate that the ALSP-associated pathology was already widely distributed throughout the brains of Csf1rΔFIRE/ΔFIRE mice. Thalamic sections of the same Csf1rΔFIRE/ΔFIRE and hCSF1R^{WT/WT} animals were then analyzed for the presence of OPN accumulation, which is another ALSP-associated pathological phenotype. As expected, thalamic sections of hCSF1R^{WT/WT} mice displayed minimal OPN immunoreactivity (FIG. 29E), whereas dense puncta of OPN accumulation was abundantly observed in thalamic sections obtained from age-matched Csf1rΔFIRE/ΔFIRE mice (FIG. 29F). Taken together, these data iMGL suggest that the CSF1R correction-based iMGL replacement therapies described in previous experiments of the disclosure mediated the reversal of pre-existing ALSP-associated pathological indications.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-159. (canceled)

160. A method of treating or preventing a leukodystrophy in a subject having a gene encoding a mutant Colony Stimulating Factor-1 Receptor (CSF1R) protein, the method comprising:
(a) obtaining a cell from a donor;
(b) producing a cell comprising a functional CSF1R protein from the cell obtained from the donor, wherein the producing comprises:
(i) repairing or replacing at least a portion of a gene encoding a mutant CSF1R protein in the cell obtained from the donor; or
(ii) culturing the cell obtained from the donor in a microglial differentiation medium for no more than 14 days; and
(c) transplanting the cell comprising the functional CSF1R protein into the subject, thereby treating or preventing the leukodystrophy in the subject.

161. The method of claim 160, wherein the cell is derived from an autologous cell source or an allogeneic cell source.

162. The method of claim 160, wherein the donor is the subject or a healthy individual.

163. The method of claim 160, wherein the cell is a human cell.

164. The method of claim 160, wherein the cell is a microglia-like cell (iMGL), a hematopoietic progenitor cell (HPC), a microglial precursor cell (MPC), myeloid cell, a hematopoietic stem cell, an erythromyeloid progenitor, myeloid precursor cell, a myeloid progenitor cell, an erythro-myeloid precursor cell, an erythro-myeloid progenitor cell, a myeloid-derived macrophage, a myeloid-derived monocyte, a myeloid-derived fetal macrophage, a non-hematopoietic stem cell-derived myeloid cell, a hematopoietic stem cell-derived myeloid cell, a yolk-sac-derived myeloid cell, a stem cell, or an induced pluripotent stem cell (iPSC).

165. The method of claim 160, wherein the cell is a microglia-like cell (iMGL).

166. The method of claim 160, wherein the cell is a human iPSC-derived microglia-like cell (iMGL).

167. The method of claim 160, wherein the culturing comprises culturing the cell obtained from the donor in a microglial differentiation medium for no more than 12 days.

168. The method of claim 160, wherein the cell does not express a mutant CSF1R gene.

169. The method of claim 160, wherein the method results in the cell comprising the functional CSF1R, and wherein the functional CSF1R protein is capable of binding to a CSF1R ligand.

170. The method of claim 160, wherein the functional CSF1R protein comprises an amino acid sequence having at least about 90% sequence identity to the amino acid sequence set forth in SEQ ID NO: 1.

171. The method of claim 160, wherein the functional CSF1R protein is encoded by a polynucleotide comprising a silent nucleotide base insertion.

172. The method of claim 160, wherein the repairing or replacing comprises contacting the gene encoding the mutant CSF1R protein with a CRISPR endonuclease, a TALEN, a zinc-finger endonuclease, a Base editor, a Prime editor, or a meganuclease.

173. The method of claim 160, wherein the repairing or replacing comprises:
(d) replacing the portion of the gene encoding the mutant CSF1R protein with a synthetic gene encoding the function CSF1R protein comprising:
(i) exons 2-21 of the functional CSF1R protein;
(ii) exons 3-21 of the functional CSF1R protein; or
(iii) exons 13-21 of the functional CSF1R protein; or
(e) repairing a mutation in the gene encoding the mutant CSF1R protein to cause the gene to encode the functional CSF1R protein.

174. The method of claim 160, wherein the leukodystrophy is Adult-onset leukoencephalopathy with axonal spheroids and pigmented glia (ALSP) or Brain Abnormalities, Neurodegeneration, and Dysosteosclerosis (BANDDOS).

175. The method of claim 160, wherein the transplanting comprises administering the cell to a central nervous system of the subject.

176. The method of claim 160, wherein the transplanting comprises administering the cell to the subject via an intracerebroventricular, intraparenchymal, intravenous, intrathecal, or intraperitoneal injection.

177. The method of claim 160, wherein, compared to a control subject, the transplanting results in:
(f) an increased number or density of Iba1-expressing microglia in a brain of the subject;
(g) an increased number, density, or average size of excitatory synapses in the brain of the subject;
(h) an increased level of expression of PSD95 or NSE in the brain of the subject;
(i) a decreased accumulation of secreted osteopontin (OPN) in the brain of the subject;
(j) a decreased frequency of axonal spheroids immunoreactive for neurofilament SMI312 and LAMP1 in the brain of the subject;
(k) a decreased level of Tau phosphorylated at Threonine-217 (pTau217) in the brain of the subject;
(l) a decreased level of GFAP expression in the brain of the subject or blood sample obtained from the subject; or
(m) a decreased level of MCP-1 expression in the brain of the subject, wherein the control subject is the subject prior to the transplanting or an individual having, or suspected of having, a leukodystrophy.

178. The method of claim 160, further comprising culturing the cell after the replacing or repairing.

179. The method of claim 160, further comprising culturing the cell before the replacing or repairing.

180. A method of preparing a cell therapy for treating or preventing leukodystrophy, the method comprising:
(n) obtaining a cell from a donor, wherein the donor is the subject or a healthy individual; and
(o) producing a cell comprising a functional CSF1R protein from the cell obtained from the doner, wherein producing comprises (i) repairing or replacing a gene encoding a mutant CSF1R protein in the cell obtained from the donor, or (ii) culturing the cell obtained from the donor in a differentiation medium for no more than 14 days,
thereby preparing the cell therapy.

181. An engineered microglia-like cell (MGL) comprising a synthetic repaired or replaced Colony Stimulating Factor 1 Receptor (CSF1R) gene.


SEQUENCE LISTING (16 sequences)

SEQ ID NO: 1 - protein, 824 aa, Homo sapiens
MRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - DNA, 3049 bp, synthetic construct
  source 1..3049: mol_type = other DNA
gtgtaggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgagcttcgagcagacatgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtactgattttgttgcgt

SEQ ID NO: 3 - DNA, 1000 bp, Homo sapiens
  source 1..1000: mol_type = genomic DNA
gcttgaatcagctctcacttccctcctttgcccctattttaggccctggaaaaatgctgacgctgcagaggcaacgggccttcttcccggacagcctgataggggtttcaagttctcttttctccttcaagaaaattttccttaaaagagattggcttcccagtaaacacagatgtgtgggggtgccggggtgagctgctgggtgtagactaggtaataaacatagtgactaactcttactgagccatttatttggtgacaggtgatgttctaaagtcttcccatgcatttaaaatgcctaacataccaatgagtgggtacgatgattgtccctgttttataggtggggaaactgaggcatggcacctccccatcccactgtgctgcagaccagatgtccattggtgggagcgggcacaccaggagattcttgggacctctctaactctgctgggctaagatcctacatctctttttttttctttcccatatgaattaagctgaggacttggccgtgaacattccattcatttgtttcttcattcggtggtagaaatacgtccactttgtacacagggttaaaagagtccattcctggggagtagaaagatggcatcacagcagggaagactgaggcaggaggctgaggaccccagggggacagaggcctgggtgagaggctgagcaagctgcaagccccctttctcagaggagggacctcctggacatcagagacatcagtctgtccctgagcaggttgagggttaggagctgagcaaatgaccagggggcaggggctcgttcaaggtggtcccttgatggcacagcaccatccctgccaagctaccacccatctcagagtcaggacggcccaaggggcgcatcctagacctcacttctgtctgctgtccctctctcccaccaggtcagggaatcccagtgatagagcccagtgtccctgagctggtcgtgaagccaggagcaacggtgaccttgcgat

SEQ ID NO: 4 - DNA, 1000 bp, Homo sapiens
  source 1..1000: mol_type = genomic DNA
gtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaaggtgaggagtctgagcctcctcccaagaggcctgacccggcaggccccactacaatgggccctaaaattaacaatcgtaacaattcagctctgcatttactgagtgctggctatgagcaaggacctggaagagctgctaatgtaatgcagtcctcacaacaaccctgcaagtcgggtctatgatgatgcattttctagaagtgcagggaggttatccaaggtcacacagcctcacatagtgggactagactggagcccaggtgcgcctgactctggagccaccacgctgaagcatccgctgaactgtcctggcgtggtgtgacctcagatgaatgatcagcctctctgagcttccttgtcacctatgtccaggtactccttggcccagtggagggagggcagttgtaaccctgtgccctcctctactctagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaaggtgcgtggggcatggggaccggcagccaggcctgaagagtggggacagagagccggcggccacatgggtggtgactggggactgggtgtgatggggggcagt

SEQ ID NO: 5 - RNA, 20 nt, synthetic construct
  source 1..20: mol_type = other RNA
aacggtgaccttgcgatgtg

SEQ ID NO: 6 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 7 - DNA, 100 bp, synthetic construct
  source 1..100: mol_type = other DNA
ccctgggatcccttcgcttacatattctttggggcaaatgcaggctgggccatttggtatccatccttcaccagtttatagaacttgctgttcaccagga

SEQ ID NO: 8 - DNA, 110 bp, synthetic construct
  source 1..110: mol_type = other DNA
gtgctttccctcagtgcatccaccgggacgtggcagcgcgtaacgtgctgttgaccaatggtcatgtggccaagattggggacttcgggctggctagggacatcatgaat

SEQ ID NO: 9 - DNA, 100 bp, synthetic construct; identical to SEQ ID NO: 7
  source 1..100: mol_type = other DNA

SEQ ID NO: 10 - DNA, 110 bp, synthetic construct
  source 1..110: mol_type = other DNA
agacaatgggcatggcctcttacctgatcttggcctttgcagggctgaatccctaccctggcatcctggtgaacagcaagttctataaactggtgaaggatggataccaa

SEQ ID NO: 11 - DNA, 100 bp, synthetic construct
  source 1..100: mol_type = other DNA
ggtgcccagggacttaagggacctgtgtgcgtggcaggtaagaccctcggagctggagcctttgggaaggtggtggaggccacggcctttggtctgggca

SEQ ID NO: 12 - RNA, 20 nt, synthetic construct
  source 1..20: mol_type = other RNA
caaatgcaggctgggcaatt

SEQ ID NO: 13 - RNA, 20 nt, synthetic construct
  source 1..20: mol_type = other RNA
cgtaacgtgctgtcgaccaa

SEQ ID NO: 14 - RNA, 20 nt, synthetic construct
  source 1..20: mol_type = other RNA
caaatgcaggctgggccgtt

SEQ ID NO: 15 - RNA, 20 nt, synthetic construct
  source 1..20: mol_type = other RNA
gcagggctgaaaccctaccc

SEQ ID NO: 16 - RNA, 20 nt, synthetic construct
  source 1..20: mol_type = other RNA
gcaggtaagaccctcgaagc